(19) United States
(12) Patent Application Publication
(10) Pub. No.: US 2024/0150452 A1
(43) Pub. Date: May 9, 2024

(54) MULTIFUNCTIONAL PROTEIN MOLECULES COMPRISING DECORIN AND USE THEREOF

(71) Applicant: Catalent Pharma Solutions, LLC, Somerset, NJ (US)

(72) Inventor: Gregory T. Bleck, Cross Plains, WI (US)

(21) Appl. No.: 18/532,395

(22) Filed: Dec. 7, 2023

Related U.S. Application Data
(63) Continuation of application No. 17/738,191, filed on May 6, 2022, now Pat. No. 11,879,009.
Application No. 17/738,191 is a division of application No. 16/457,045, filed on Jun. 28, 2019, now Pat. No. 11,377,488.
(60) Provisional application No. 62/693,766, filed on Jul. 3, 2018.

(51) Int. Cl. C07K 16/22; A61P 35/00; C07K 14/47; C07K 16/28

(52) U.S. Cl. CPC C07K 16/22; A61P 35/00; C07K 14/4725; C07K 16/2818; C07K 16/2827; C07K 2317/24; C07K 2317/56

(57) ABSTRACT

The present invention relates to multifunctional protein molecules comprising decorin and uses thereof. In particular, the present invention relates to multifunctional protein molecules comprising decorin and a targeting polypeptide such as an antibody and methods of their production and uses thereof.

This application is a continuation of U.S. patent application Ser. No. 17/738,191, filed May 6, 2022, which is a divisional of U.S. patent application Ser. No. 16/457,045, filed Jun. 28, 2019, issued as U.S. Pat. No. 11,377,488, which claims priority to U.S. Provisional Application No. 62/693,766, filed Jul. 3, 2018, the entire contents of which are incorporated herein by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 116,798 Byte ASCII file named “CATA_33890_304_SequenceListing.xml” created on Dec. 7, 2023.

### FIELD OF THE INVENTION

The present invention relates to multifunction protein molecules comprising decorin and uses thereof. In particular, the present invention relates to multifunctional protein molecules comprising decorin and a targeting polypeptide such as an antibody and methods of their production and uses thereof.

### BACKGROUND OF THE INVENTION

VEGF

Angiogenesis is the physiological process through which new blood vessels form from pre-existing vessels. This is distinct from vasculogenesis, which is the de novo formation of endothelial cells from mesoderm cell precursors. The first vessels in the developing embryo form through vasculogenesis, after which angiogenesis is responsible for most, if not all, blood vessel growth during development and in disease.

Angiogenesis is a normal and vital process in growth and development, as well as in wound healing and in the formation of granulation tissue. However, it is also a fundamental step in the transition of tumors from a benign state to a malignant one, leading to the use of angiogenesis inhibitors in the treatment of cancer.

Vascular endothelial growth factor (VEGF), originally known as vascular permeability factor (VPF), is a signal protein produced by cells that stimulates vasculogenesis and angiogenesis. It is part of the system that restores the oxygen supply to tissues when blood circulation is inadequate. Serum concentration of VEGF is high in bronchial asthma and diabetes mellitus. VEGF's normal function is to create new blood vessels during embryonic development, new blood vessels after injury, muscle following exercise, and new vessels (collateral circulation) to bypass blocked vessels.

When VEGF is overexpressed, it can contribute to disease. Solid cancers cannot grow beyond a limited size without an adequate blood supply; cancers that can express VEGF are able to grow and metastasize. Overexpression of VEGF can cause vascular disease in the retina of the eye and other parts of the body. Drugs such as bevacizumab and ranibizumab can inhibit VEGF and control or slow those diseases.

VEGF is a sub-family of growth factors, to be specific, the platelet-derived growth factor family of cystine-knot growth factors. They are important signaling proteins involved in both vasculogenesis (the de novo formation of the embryonic circulatory system) and angiogenesis (the growth of blood vessels from pre-existing vasculature).

Checkpoint Inhibitors

An important part of the immune system is its ability to tell between normal cells in the body and those it sees as “foreign.” This lets the immune system attack the foreign cells while leaving the normal cells alone. To do this, it uses “checkpoints”—molecules on certain immune cells that need to be activated (or inactivated) to start an immune response. Cancer cells sometimes find ways to use these checkpoints to avoid being attacked by the immune system. But drugs that target these checkpoints hold a lot of promise as cancer treatments.

Checkpoint inhibitors seek to overcome one of cancer's main defenses against an immune system attack. Immune system T cells patrol the body constantly for signs of disease or infection. When they encounter another cell, they probe certain proteins on its surface, which serve as insignia of the cell's identity. If the proteins indicate the cell is normal and healthy, the T cell leaves it alone. If the proteins suggest the cell is infected or cancerous, the T cell will lead an attack against it. Once T cells initiate an attack, the immune system increases a series of additional molecules to prevent the attack from damaging normal tissues in the body. These molecules are known as immune checkpoints.

Checkpoint inhibitors block these normal proteins on cancer cells, or the proteins on T cells that respond to them. The result is to remove the blinders that prevented T cells from recognizing the cells as cancerous and leading an immune system assault on them. Three checkpoint inhibitors have received rapid approval from the U.S. Food and Drug Administration for cancer, including ipilimumab (Yervoy®), pembrolizumab (Keytruda®), and nivolumab (Opdivo®). These and other immune checkpoint therapies represent one of the most promising frontiers in cancer treatment today.

Additional therapeutic agents that target VEGF or immune checkpoints are needed.

### SUMMARY OF THE INVENTION

The present invention relates to multifunction protein molecules comprising decorin and uses thereof. In particular, the present invention relates to multifunctional protein molecules comprising decorin and a targeting polypeptide such as an antibody and methods of their production and uses thereof.

Accordingly, in some embodiments, the present invention provides multifunctional protein molecules comprising at least one decorin molecule or functional portion thereof linked to an antigen binding protein.

In some preferred embodiments, the antigen binding protein is selected from the group consisting of a VEGF-A antigen binding protein and a checkpoint inhibitor antigen binding protein. In some preferred embodiments, the checkpoint inhibitor antigen binding protein binds a checkpoint inhibitor protein selected from the group consisting of PD-1, PD-L1, CTLA-4, PD-L2, CD27, CD28, CD40, CD47, CD115, CD122, CD137, OX40, GITR, ICOS, A2AR, B7-H3, B7-H4, BTLA, IDO, KIR, LAG3, NOX2, TIM-3, VISTA, SIGLEC-7, TIGIT and 4-1BB. In some particularly preferred embodiments, the checkpoint inhibitor antigen binding protein binds a checkpoint inhibitor protein selected from the group consisting of PD-1, PD-L1, CTLA-4, PD-L2.

In some preferred embodiments, the antigen binding protein is an antibody. In some preferred embodiments, the antibody is a monoclonal antibody. In some preferred embodiments, the monoclonal antibody is selected from the group consisting of bevacizumab, ranibizumab, ipilimumab, atezolizumab, avelumab, durvalumab, nivolumab and pembrolizumab.

In some preferred embodiments, the decorin polypeptide is a decorin core protein. In some preferred embodiments, the decorin core protein comprises a mutation at position 4 of the mature decorin core protein. In some preferred embodiments, the mutation is a serine to alanine mutation. In some preferred embodiments, the decorin core protein lacks substantial modification by glycosaminoglycans molecules at position 4 of the mature decorin core protein. In some preferred embodiments, the fusion protein comprises two or more copies of the decorin polypeptide.

In some preferred embodiments, the at least a functional portion of a decorin molecule comprises a decorin domain or domains that binds a signaling molecule selected from the group consisting of Transforming Growth Factor-β (TGF-β), Connective Tissue Growth Factor (CTGF), Platelet-Derived Growth Factor (PDGF), Vascular Endothelial Growth Factor Receptor 2 (VEGFR2), Hepatocyte Growth Factor Receptor (HGFR), Insulin-like Growth Factor 1 Receptor (IGF-1R), Epidermal Growth Factor Receptors (EGFRs), myostatin and Clq. In some preferred embodiments, the TGF-β binding domain comprises amino acids Asp45-Lys359 of full length endogenous human decorin or amino acids Leu155-Va1260 of full length endogenous human decorin. In some preferred embodiments, the multifunctional protein molecule comprises two or more copies of the at least a functional portion of a decorin molecule.

In some preferred embodiments, the decorin molecule is operably linked to a heavy chain of the antibody. In some preferred embodiments, the antigen binding protein is bi-specific. In some preferred embodiments, the antigen binding protein is multi-specific.

In some preferred embodiments, the multifunctional protein molecule is a fusion protein. In some preferred embodiments, the decorin molecule is chemically linked to the antigen binding protein.

In some preferred embodiments, the present invention provides a nucleic acid or set of nucleic acids encoding a multifunctional protein molecule as described above. In some preferred embodiments, the present invention provides a vector or vectors comprising the nucleic acid or set of nucleic acids. In some preferred embodiments, the present invention provides a host cell comprising the vector or vectors.

In some preferred embodiments, the present invention provides methods of inhibiting a target protein and a signaling molecule in a cell, comprising: contacting the cell with the multifunctional protein molecule, nucleic acid molecule, or vector as described above under conditions such that at least one activity of the target protein and at least one activity of a signaling protein are inhibited in the cell, wherein the target protein is selected from the group consisting of VEGF-1, PD-1, PD-L1, CTLA-4, PD-L2, CD27, CD28, CD40, CD47, CD115, CD122, CD137, OX40, GITR, ICOS, A2AR, B7-H3, B7-H4, BTLA, IDO, KIR, LAG3, NOX2, TIM-3, VISTA, SIGLEC-7, TIGIT and 4-1BB. In some preferred embodiments, the cell is in vitro or in vivo. In some preferred embodiments, the cell is in a subject. In some preferred embodiments, the contacting results in inhibition of an activity selected from the group consisting of angiogenesis, PD-1 activity, PD-L1 activity, CTLA-4 activity, PD-L2 activity, CD27 activity, CD28 activity, CD40 activity, CD47 activity, CD115 activity, CD122 activity, CD137 activity, OX40 activity, GITR activity, ICOS activity, A2AR activity, B7-H3 activity, B7-H4 activity, BTLA activity, IDO activity, KIR activity, LAG3 activity, NOX2 activity, TIM-3 activity, VISTA activity, SIGLEC-7 activity, TIGIT activity and 4-1BB activity. In some preferred embodiments, the cancer is selected from the group consisting of lung cancer, colorectal cancer, liver cancer, breast cancer, kidney cancer, cervical cancer, ovarian cancer, and glioblastoma. In some preferred embodiments, the signaling protein is selected from the group consisting of Transforming Growth Factor-β (TGF-β), Connective Tissue Growth Factor (CTGF), Platelet-Derived Growth Factor (PDGF), Vascular Endothelial Growth Factor Receptor 2 (VEGFR2), Hepatocyte Growth Factor Receptor (HGFR), Insulin-like Growth Factor 1 Receptor (IGF-1R), various epidermal growth factor receptors (EGFRs), myostatin and Clq. In some preferred embodiments, the signaling protein is Transforming Growth Factor-(3 (TGF-0).

In some preferred embodiments, the present invention provides methods of treating a disorder characterized by angiogenesis or tumor growth, comprising: administering a multifunctional protein molecule, nucleic acid molecule, or vector as described above to a subject under conditions such that angiogenesis or tumor growth is inhibited in the subject. In some preferred embodiments, the tumor is selected from the group consisting of lung cancer, colorectal cancer, liver cancer, breast cancer, kidney cancer, cervical cancer, ovarian cancer, and glioblastoma.

### DESCRIPTION OF THE FIGURES

FIG. 1 is a schematic diagram of a fusion protein of the present invention.

FIG. 2 is a map of an expression construct of the present invention.

FIG. 3 is a map of an expression construct of the present invention.

FIG. 4 is a SDS-PAGE gel of media from the pooled CHO cell line expressing the bevacizumab fusion protein. Lane 10. Molecular weight standards. Lane 11. Non-reduced media sample. Lane 12. Reduced media sample.

FIG. 5 is a SDS-PAGE gel of purified Avelumab-Galacorin fusion molecule. Lane 1: Molecular weight markers. Lane 2: Non-reduced purified Avelumab-Galacorin fusion. Lane 3: Reduced purified Avelumab-Galacorin fusion.

FIG. 6 is a SEC-HPLC chromatogram of purified Avelumab-Galacorin fusion molecule. Percent monomer was greater than 98%.

FIG. 7 is a graph of tumor growth in a C57BL/6 mouse MC-38 human colorectal cancer model after a single IV dose of the four treatments.

### DEFINITIONS

To facilitate understanding of the invention, a number of terms are defined below.

As used herein, the term “decorin” refers to a protein molecule having a mature protein sequence that is at least 80% identical to SEQ ID NOs: 1, 2, or 6 or a portion thereof.

As used herein, the term “decorin core protein” refers to a decorin protein molecule that has a mutation at amino acid 4 of mature decorin and that substantially lacks modification with a glycosaminoglycan (GAG; i.e., is non-gagylated) at amino acid 4.

As used herein, the term “multifunctional protein molecule” refers to protein molecules that comprises two or more polypeptide sub-portions derived from at least two different sources. The multifunctional protein molecules may be recombinant fusion proteins encoded by a fusion gene or may be made by chemical addition (e.g., by covalent modification) of a polypeptide to another polypeptide. For example, fusion proteins of the present invention may preferably comprise one or more decorin molecules or functional portions thereof linked to an antigen binding protein via a linker sequence or may be “chemical fusions” wherein one or more decorin molecules or functional portions thereof are covalently linked to an antigen binding protein, for example, via a modified amino acid.

As used herein, the term “host cell” refers to any eukaryotic cell (e.g., mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “vector” refers to any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The terms “homology” and “percent identity” when used in relation to nucleic acids refers to a degree of complementarity. There may be partial homology (i.e., partial identity) or complete homology (i.e., complete identity). A partially complementary sequence is one that at least partially inhibits a completely complementary sequence from hybridizing to a target nucleic acid sequence and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe (i.e., an oligonucleotide which is capable of hybridizing to another oligonucleotide of interest) will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the term “signal sequence” refers to any DNA sequence which, when operably linked to a recombinant DNA sequence, encodes a signal peptide which is capable of causing the secretion of the recombinant polypeptide. In general, the signal peptides comprise a series of about 15 to 30 hydrophobic amino acid residues (See, e.g., Zwizinski et al., J. Biol. Chem. 255(16): 7973-77 [1980], Gray et al., Gene 39(2): 247-54 [1985], and Martial et al., Science 205: 602-607 [1979]). Such secretion signal sequences are preferably derived from genes encoding polypeptides secreted from the cell type targeted for tissue-specific expression (e.g., secreted milk proteins for expression in and secretion from mammary secretory cells). Secretory DNA sequences, however, are not limited to such sequences. Secretory DNA sequences from proteins secreted from many cell types and organisms may also be used (e.g., the secretion signals for t-PA, serum albumin, lactoferrin, and growth hormone, and secretion signals from microbial genes encoding secreted polypeptides such as from yeast, filamentous fungi, and bacteria).

As used herein, the term “purified” refers to molecules, either nucleic or amino acid sequences, that are removed from their normal environment, isolated or separated. An “isolated nucleic acid sequence” is therefore a purified nucleic acid sequence. “Substantially purified” molecules are at least 60% free, preferably at least 75% free, and more preferably at least 90% free from other components with which they are normally associated.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “antibody” is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “epitope” refers to the particular site on an antigen molecule to which an antibody binds.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, MD (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated antibody” is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W. H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### DETAILED DESCRIPTION OF THE INVENTION

The present invention relates to multifunctional molecules comprising a decorin molecule or functional portion thereof operably linked to an antigen binding protein such as an immunoglobulin molecule, truncated fragment of an antigen molecule, or a single chain antibody. In particular, the present invention relates to fusion polypeptides comprising decorin and a VEGF or immune checkpoint targeting polypeptide (preferably a checkpoint inhibitor) and methods of their production and uses thereof. Embodiments of the present invention provide fusion polypeptides comprising a decorin polypeptide fused to an antigen binding protein of interest, nucleic acids encoding such polypeptides, and uses thereof. Exemplary compositions and methods are described herein. The decorin utilized in the fusion molecules may be wild type decorin, decorin core protein, or functional portions of either of these proteins, such as the portion(s) that bind TGF-β, or other signaling molecules such as Connective Tissue Growth Factor (CTGF), Platelet-Derived Growth Factor (PDGF), Vascular Endothelial Growth Factor Receptor 2 (VEGFR2), Hepatocyte Growth Factor Receptor (HGFR), Insulin-like Growth Factor 1 Receptor (IGF-1R), various epidermal growth factor receptors (EGFRs), myostatin and Clq.

### I. Decorin

In preferred embodiments, multifunctional protein molecules of the present invention comprise one or more decorin polypeptides or functional portions thereof. Decorin has been shown to suppress transforming growth factor-beta-induced expression of plasminogen activator inhibitor-1 (See e.g., Wahab et al., Biochem J. 2002 Mar. 15; 362(Pt 3): 643-649; herein incorporated by reference in its entirety). Transforming growth factor-beta (TGF-β) is a key mediator of extracellular matrix (ECM) accumulation in sclerotic kidney diseases such as diabetic nephropathy. While not being limited to a particular mechanism, it is contemplated that the combination of decorin and a VEGF binding protein will improve efficacy over the VEGF binding protein by itself, through inhibiting TGF-β activity in combination with the blockage of VEGF.

Native decorin is a glycoprotein with an attached glycosaminoglycan and an average molecular weight of 90-140 kD. In some preferred embodiments, the decorin is decorin core protein, i.e., a substantially non-gagylated decorin. In some embodiments, the decorin core protein comprises a mutation at amino acid 4 (i.e., the 4^{th} amino acid from the N-terminus) of the mature decorin core protein molecule. In some embodiments, the mutation is a serine to alanine mutation. In some embodiments, the decorin core protein is at least 90%, 95%, 99% or 100% identical to SEQ ID NO:6 (mature decorin core protein), provided that that the decorin core protein comprises a mutation at amino acid 4 (i.e., the 4^{th} amino acid from the N-terminus) of the mature decorin core protein molecule.

Decorin is commonly expressed as a pre-pro-protein. The present invention provides multifunctional protein molecules comprising an antigen binding protein in operable association with one or more decorin mature peptide sequences or functional portions thereof. In some embodiments, the decorin core protein portion of the fusion polypeptide is at least 90%, 95%, 99% or 100% identical to SEQ ID NO:6 (mature decorin core protein) or a functional portion thereof. In some embodiments, the decorin core protein comprises a mutation at amino acid 4 (i.e., the 4^{th} amino acid from the N-terminus) of the mature decorin core protein molecule.

The present invention further provides nucleic acid sequences encoding fusion proteins, as well as vectors comprising the nucleic acid sequences. In some embodiments, the decorin core protein portion of the fusion polypeptide is at least 90%, 95%, 99% or 100% identical to SEQ ID NO:5 (mature decorin core protein), provided that that the decorin core protein comprises a mutation at amino acid 4 (i.e., the 4^{th} amino acid from the N-terminus) of the mature decorin core protein molecule.

In some embodiments, the decorin molecule utilized in the multifunctional protein molecules may comprise one or more functional portions of the decorin molecule. Decorin molecules have a number of functional portions or domains, for example, those described in Jarvinen and Prince, BioMed Research Int'l, Vol. 2015, Article ID 654765 (incorporated by reference herein in its entirety). In some preferred embodiments, the decorin functional portion binds to or otherwise interacts with Transforming Growth Factor-β (TGF-β), Connective Tissue Growth Factor (CTGF), Platelet-Derived Growth Factor (PDGF), Vascular Endothelial Growth Factor Receptor 2 (VEGFR2), Hepatocyte Growth Factor Receptor (HGFR), Insulin-like Growth Factor 1 Receptor (IGF-1R), various epidermal growth factor receptors (EGFRs), myostatin or Clq. Preferred decorin functional portions retain functional activity (such as binding to one of the signaling molecules just described) and are preferably at least 90%, 95%, 99% or 100% identical to the corresponding native decorin sequence. Preferred decorin functional portions are shorter than the full-length native decorin molecule, and for example, may be from 10 to 300 amino acid in length or from 10 to 120 amino acids in length. For example, the functional portion of the decorin molecule may be at least 90%, 95%, 99% or 100% identical to SEQ ID NO:64 (decorin TGF-β binding domain, ASP45-LYS359 of full length endogenous human decorin), SEQ ID NO:65 (two copies decorin TGF-β binding domain, ASP45-LYS359, separated by linker), SEQ ID NO:66 (decorin TGF-β binding domain, LEU155-VAL260 of full length endogenous human decorin), SEQ ID NO:67 (two copies decorin TGF-β binding domain, LEU155-VAL260, separated by linker).

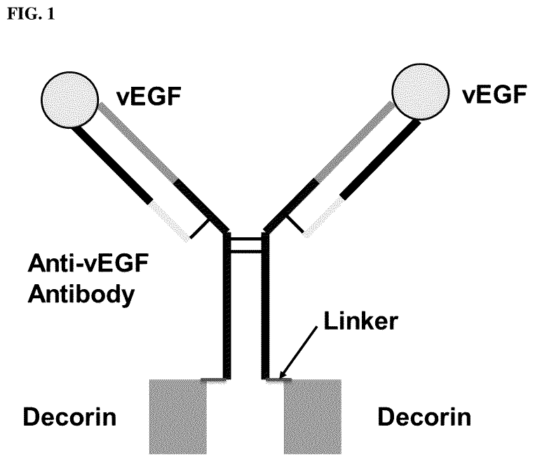

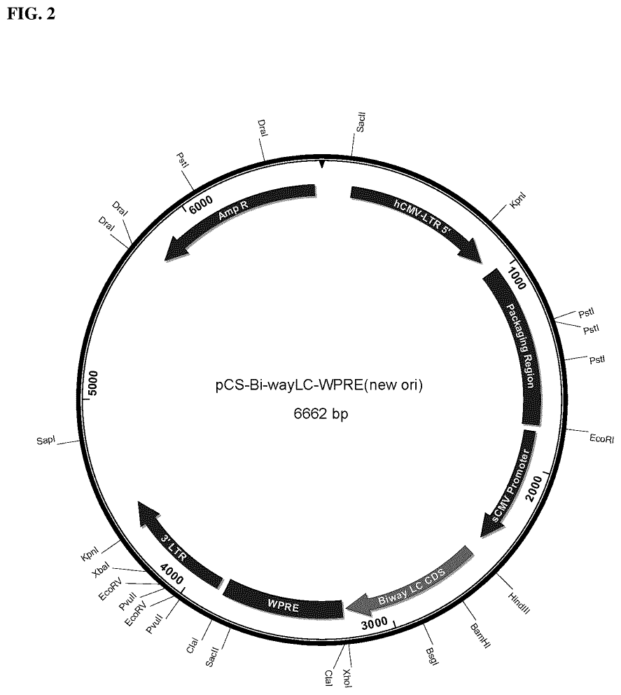

Exemplary decorin polypeptides and method of purifying decorin are described, for example, in WO 2006038107; herein incorporated by reference in its entirety. Exemplary decorin nucleic acid and amino acid sequences are provided below and in FIGS. 1 and 2.

| SEQ ID NO. | Sequence |
|---|---|
| SEQ ID NO: 46 | DEAAGIGPEVPDDRDFEPSLGPVCPFRCQ |
| Decorin core protein | CHLRVVQCSDLGLDKVPKDLPPDTTLLD |
| | LQNNKITEIKDGDFKNLKNLHALILVNNK |
| | ISKVSPGAFTPLVKLERLYLSKNQLKELPE |
| | KMPKTLQELRAHENEITKVRKVTFNGLN |
| | QMIVIELGTNPLKSSGIENGAFQGMKKLS |
| | YIRIADTNITSIPQGLPPSLTELHLDGNKIS |
| | RVDAASLKGLNNLAKLGLSFNSISAVDN |
| | GSLANTPHLRELHLDNNKLTRVPGGLAE |
| | HKYIQVVYLHNNNISVVGSSDFCPPGHNT |
| | KKASYSGVSLFSNPVQYWEIQPSTFRCVY |
| | VRSAIQLGNYK |
| SEQ ID NO: 47 | GPFQQRGLFDFMLE |
| Decorin propeptide | |
| SEQ ID NO: 48 | GGCCCGTTTCAACAGAGAGGCTTATTTG |
| Decorin propeptide | ACTTTATGCTAGAA |
| SEQ ID NO : 49 | GATGAGGCTGCAGGGATAGGCCCAGAA |
| Decorin core protein | GTTCCTGATGACCGCGACTTCGAGCCCT |
| | CCCTAGGCCCAGTGTGCCCCTTCCGCTG |
| | TCAATGCCATCTTCGAGTGGTCCAGTGT |
| | TCTGATTTGGGTCTGGACAAAGTGCCA |
| | AAGGATCTTCCCCCTGACACAACTCTGC |
| | TAGACCTGCAAAACAACAAAATAACCG |
| | AAATCAAAGATGGAGACTTTAAGAACC |
| | TGAAGAACCTTCACGCATTGATTCTTGT |
| | CAACAATAAAATTAGCAAAGTTAGTCC |
| | TGGAGCATTTACACCTTTGGTGAAGTTG |
| | GAACGACTTTATCTGTCCAAGAATCAG |
| | CTGAAGGAATTGCCAGAAAAAATGCCC |
| | AAAACTCTTCAGGAGCTGCGTGCCCAT |
| | GAGAATGAGATCACCAAAGTGCGAAAA |
| | GTTACTTTCAATGGACTGAACCAGATG |
| | ATTGTCATAGAACTGGGCACCAATCCG |
| | CTGAAGAGCTCAGGAATTGAAAATGGG |
| | GCTTTCCAGGGAATGAAGAAGCTCTCC |
| | TACATCCGCATTGCTGATACCAATATCA |
| | CCAGCATTCCTCAAGGTCTTCCTCCTTC |
| | CCTTACGGAATTACATCTTGATGGCAAC |
| | AAAATCAGCAGAGTTGATGCAGCTAGC |
| | CTGAAAGGACTGAATAATTTGGCTAAG |
| | TTGGGATTGAGTTTCAACAGCATCTCTG |
| | CTGTTGACAATGGCTCTCTGGCCAACAC |
| | GCCTCATCTGAGGGAGCTTCACTTGGAC |
| | AACAACAAGCTTACCAGAGTACCTGGT |
| | GGGCTGGCAGAGCATAAGTACATCCAG |
| | GTTGTCTACCTTCATAACAACAATATCT |
| | CTGTAGTTGGATCAAGTGACTTCTGCCC |
| | ACCTGGACACAACACCAAAAAGGCTTC |
| | TTATTCGGGTGTGAGTCTTTTCAGCAAC |
| | CCGGTCCAGTACTGGGAGATACAGCCA |
| | TCCACCTTCAGATGTGTCTACGTGCGCT |
| | CTGCCATTCAACTCGGAAACTATAAGT |
| | GA |
| SEQ ID NO: 64 | 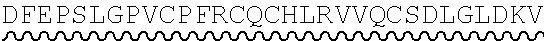 |
| Decorin TGF-β binding domain ASP45- | 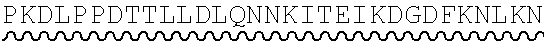 |
| LYS359 | 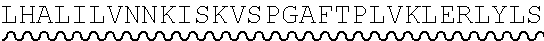 |
| | 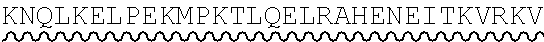 |
| | 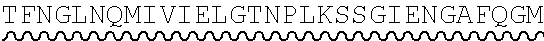 |
| | 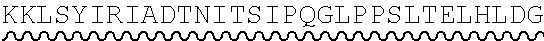 |
| | 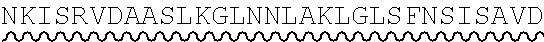 |
| | 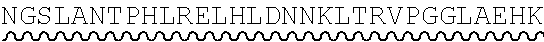 |
| | 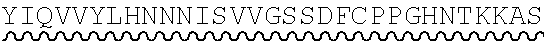 |
| | 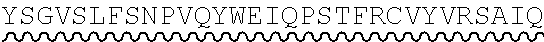 |
| | 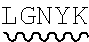 |
| SEQ ID NO: 65 Decorin TGF-fβ binding | 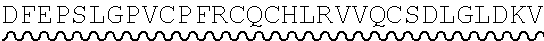 |
| domain A5P45-LY5359, two copies | 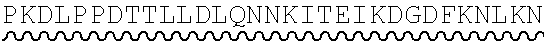 |
| separated by linker | 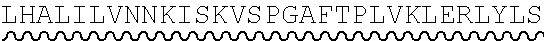 |
| | 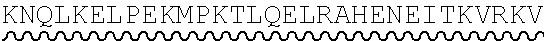 |
| | 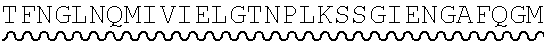 |
| | 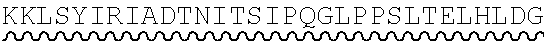 |
| | 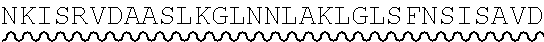 |
| | 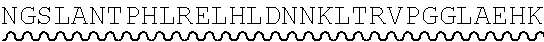 |
| | 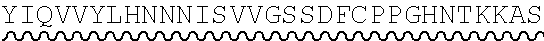 |
| | 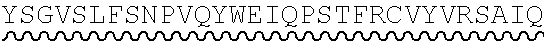 |
| | 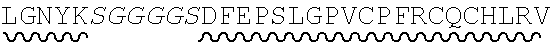 |
| | 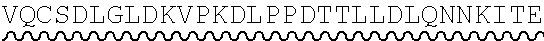 |
| | 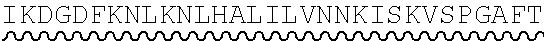 |
| | 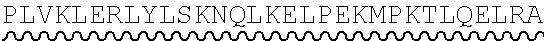 |
| | 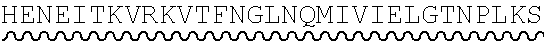 |
| | 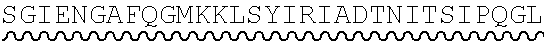 |
| | 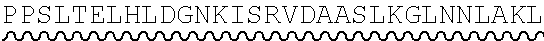 |
| | 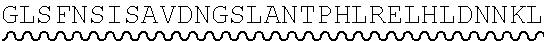 |
| | 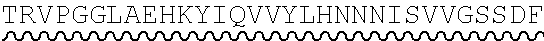 |
| | 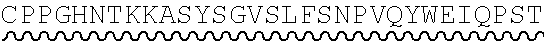 |
| | 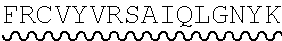 |
| SEQ ID NO: 66 | 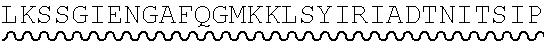 |
| Decorin TGF-β binding domain LEU155- | 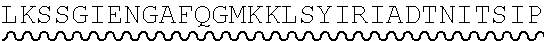 |
| VAL260 | 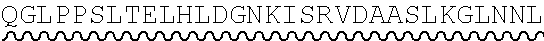 |
| | 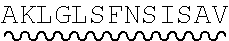 |
| SEQ ID NO: 67 | 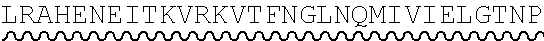 |
| Decorin TGF-β binding domain LEU155- | 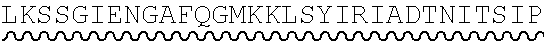 |
| VAL260 | 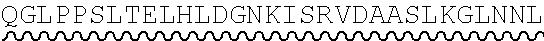 |
| Two copies separated by linker | 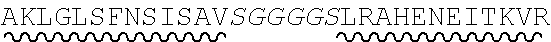 |
| | 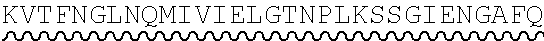 |
| | 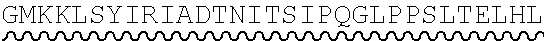 |
| | 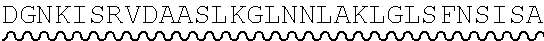 |
| | 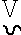 |

### II. Binding Agents

Preferred embodiments of the present invention provide multifunctional protein molecules comprising a one or more decorin molecules or functional portion(s) thereof that are operably linked to a binding agent of interest. Preferred binding agents of interest include, but are not limited to, antigen binding proteins including immunoglobulins and fragments or derivatives thereof such as single chain antibodies that bind to molecules such as VEGF (Vascular endothelial growth factor) and checkpoint inhibitor proteins such asCTLA-4 (Cytotoxic T-lymphocyte antigen 4), PD-1 (Programmed cell death protein 1), PD-L1 (Programmed death-ligand 1), PD-L2 (Programmed death-ligand 2), CD27, CD28, CD40, CD47, CD115, CD122, CD137, OX40, GITR, ICOS, A2AR, B7-H3, B7-H4, BTLA, IDO, KIR, LAG3, NOX2, TIM-3, VISTA, SIGLEC-7, TIGIT and 4-1BB.

Binding agents (e.g., antigen binding proteins) generally interact with or bind specifically with a target. For example, the binding agents disclosed herein generally interact specifically with regions of, for example, VEGF, CTLA-4, PD-1 or PD-1, which are collectively referred to herein as target proteins. Binding “specifically” to a target protein means that the amount of binding to the target protein is more than the amount of binding to non-target protein targets (e.g., there may be background nonspecific binding). Generally, specific binding of binding agents to a protein, for example, may be achieved by binding to a specific sequence of amino acids within a protein target. These sequences may be referred to as epitopes. Molecules containing the epitopes may be used to stimulate binding agents like antibodies and may be referred to as immunogens. The binding agents may also recognize specific 2- and/or 3-dimensional structures as part of the epitope. Antigen binding protein may be mono-specific, bi-specific, or multispecific.

The specific interaction or binding of a binding agent with its target is thought to be a type of equilibrium reaction. In one example, the specific binding can be quantified. The quantification may use a dissociation constant, or Kd is known in the art to be a type of equilibrium constant that describes the propensity of, in this case, an antibody to separate from the antigen or epitope to which it has bound. Thus, Kd describes the affinity that an antibody has for an epitope. The lower the Kd, the higher is the affinity of a binding agent for its target.

In certain embodiments, the binding agent is a monoclonal antibody. The antibody (e.g., monoclonal antibody) may also be of any suitable isotype or isotype subclass. The binding agent may also be a derivative of an antibody such as, for example, a Fab, F(ab′)2, Fab′ single chain antibody, Fv, single chain, mono-specific antibody, bi-specific antibody, tri-specific antibody, multi-valent antibody, chimeric antibody, humanized antibody, human antibody, shark antibody, nanobody (e.g., antibody comprising a single monomeric variable domain), camelid antibody (e.g., from the Camelidae family) microbody, intrabody (e.g., intracellular antibody), or de-fucosylated antibody and/or derivative thereof. Mimetics of binding agents and/or antibodies are also contemplated as being within the present invention. The binding agent may also comprise a detectable label and/or effector moiety attached thereto.

Where the binding agent is an antigen binding protein such as an immunoglobulin or derivative thereof, it may be identified with reference to the nucleotide and/or amino acid sequence corresponding to the variable and/or complementarity determining regions (“CDRs”) thereof. For instance, an exemplary binding agent that is, is derived from, or is related to the monoclonal antibodies described herein may comprise a heavy and/or a light chain that each comprise one or more constant and/or variable regions. The variable regions typically comprise one or more CDRs that in large part determine the binding specificity of the antibody. These monoclonal antibodies may be identified by analysis of the nucleotide sequences encoding the variable regions. The monoclonal antibodies may also be identified by analysis of the amino acid sequences of (e.g., encoded by the nucleotide sequences) the variable regions.

The amino acids in the multifunctional protein molecules of the present invention may also be substituted by any other amino acid as desired by one of ordinary skill in the art. For example, one of skill in the art may make conservative substitutions by replacing particular amino acids with others as is known in the art. Any of the amino acid sequences of the antigen binding proteins described herein may also be combined with any other variable region and/or CDR in any order and/or combination to form hybrid and/or fusion binding agents and/or inserted into other heavy and/or light chain variable regions using standard techniques. These may be used in conjunction with any constant regions.

CDRs (complementarity-determining regions) are amino acid sequences from antibodies that are, at least in part, responsible for binding of an antibody to a specific target. It is understood by those of skill in the art that CDRs may be identified using any of several techniques and/or schemes. CDRs of the binding agents shown herein may be identified using any of these techniques. For instance, one of ordinary skill in the art may identify CDRs using the Kabat Numbering Scheme, the Chothia Numbering Scheme, the Enhanced Chothia Numbering Scheme, and/or any of the available CDR Definition Schemes (e.g., AbM, contact definition, and/or as described by MacCullum, et al., J Mol. Biol,. 262(5):732-745, 1996. A summary of various schemes, in part based on, for example, Kabat et al., “Sequences of Proteins of Immunological Interest,” 5^{th} Ed., Public Health Service, National Institutes of Health, Bethesda, MD, NIH publication No. 91-3242 (1991), and Al-Lazikani et al., “Standard conformations for the canonical structures of immunoglobulins,” J.Mol.Biol. 273:927-948, 1997,

These systems for identifying CDRs are merely exemplary and others may be suitable, as would be understood by one of ordinary skill in the art. CDRs thus identified may be used to identify suitable binding agents. For instance, equivalents of one or more of the monoclonal antibodies described herein. Such CDRs may also be combined with one another in any order and/or combination to form hybrid and/or fusion binding agents and/or inserted into the other heavy and/or light chain variable regions using standard techniques.

In some embodiments, the CDR sequences from antigen binding proteins described herein are joined to the constant regions of any antibody molecule of the same or a different species (e.g., human, goat, rat, sheep, chicken) of that from which the variable region amino acid sequence was derived.

Deamidation of asparagine residues to aspartic acid or isoaspartic acid is a common post-translational modification to proteins. Deamidation may occur with higher frequency when the asparagine is part of an asparagine-glycine dipeptide (Asp-Gly or N-G; the “NG” sequence). Deamidation may have detrimental effects on proteins. In one example, deamidation may potentially cause a change in the three-dimensional structure of a protein. In another example, for an antibody, deamidation in a region that affects binding to an antigen (e.g., variable regions and/or CDRs) may potentially cause lower or loss of antibody binding to the antigen.

Accordingly, in some embodiments, amino acid residues potentially susceptible to post-translational deamidation are substituted with those less or not susceptible. In one example, asparagine and/or glycine is substituted to modify the NG sequence for example, any amino acid that eliminates the NG sequence.

The constant regions of antibodies are derived from any of, for example, human (e.g., IgG (IgG1, IgG2, IgG3, IgG4), IgM, IgA (IgA1 and IgA2), IgD, and IgE), canine (e.g., IgG (IgGA, IgGB, IgGC, IgGD) IgA, IgD, IgE, and IgM), chicken (e.g., IgA, IgD, IgE, IgG, IgM, IgY), goat (e.g., IgG), mouse (e.g., IgA, IgG, IgD, IgE, IgM), pig (e.g., IgA, IgG, IgD, IgE, IgM), rat (e.g., IgA, IgG, IgD, IgE, IgM), feline (e.g., IgA, IgD, IgE, IgG, IgM) and/or a fragment and/or derivative thereof (e.g., as chimeric antibodies).

In one example, the binding agents are antibodies that have modified glycosylation patterns. IgG molecules, for example, typically contain N-linked oligosaccharides. Some IgG molecules contain a biantennary complex-type oligosaccharide linked to the antibody heavy chain. In human IgG, the oligosaccharide is generally linked to an asparagine residue at position 297 (N297) of the heavy chain (in the constant Fc region of the antibody heavy chain). Generally, a fucose is attached to the GLcNAC residue in the oligosaccharide that is nearest to N297. Absence of the fucose may enhance the ability of the antibodies to mediate antibody-dependent cellular cytotoxicity (ADCC). It is contemplated that removal of the fucose enhances the ability of the antibody to interact with Fc receptors. Antibodies of this type are referred to as “defucosylated”. Defucosylated antibodies may be produced using techniques described herein that may be known in the art. In some embodiments, a nucleic acid sequence encoding an antibody may be expressed in a cell line that has modified glycosylation abilities (e.g., deleted, modified or lesser amount of fucosyl transferase) and fail to add the typical fucose moieties. A variety of these cell lines are known. In some embodiments, the antibodies disclosed herein bind to VEGF but contain defucosylated oligosaccharides. The binding agents (e.g., antibodies) may include other modifications that may result in decreased interaction with Fc receptors. For instance, alternative or additional amino acid substitutions may be made to the antibody molecules described herein.

As described above, in some embodiments, the binding agents may be antibodies or immunoglobulins. The term “antibody” or “antibodies” may refer to whole or fragmented antibodies in unpurified or partially purified form (e.g., hybridoma supernatant, ascites, polyclonal antisera) or in purified form. A “purified” antibody may be one that is separated from at least about 50% of the proteins with which it is initially found (e.g., as part of a hybridoma supernatant or ascites preparation). A purified antibody may be one that is separated from at least about 60%, 75%, 90%, or 95% of the proteins with which it is initially found. Suitable derivatives may also be fragments (e.g., Fab, F(ab′)2 or single chain antibodies, like Fv, for example). The antibodies may be of any suitable origin or form including, for example, murine (e.g., produced by murine hybridoma cells), or expressed as chimeric antibodies, and the like.

Methods of preparing and utilizing various types of antibodies are well-known to those of skill in the art and would be suitable in practicing the present invention (see, for example, Harlow, et al. Antibodies: A Laboratory Manual, Cold Spring Harbor Laboratory, 1988; Harlow, et al., Using Antibodies: A Laboratory Manual, Portable Protocol No. 1, 1998; Kohler and Milstein, Nature, 256:495, 1975; Jones et al., Nature, 321:522-525, 1986; Riechmann et al., Nature, 332:323-329, 1988; Presta, Curr. Op. Struct. Biol.,2:593-596, 1992; Verhoeyen et al., Science, 239:1534-1536, 1988; Hoogenboom et al., J Mol. Biol., 227:381, 1991; Marks et al., J Mol. Biol., 222:581, 1991; Cole et al., Monoclonal Antibodies and Cancer Therapy, Alan R. Liss, p. 77, 1985; Boerner et al., J Immunol., 147(1):86-95, 1991; Marks et al., BioiTechnology 10, 779-783, 1992; Lonberg et al., Nature 368:856-859, 1994; Morrison, Nature 368:812-13, 1994; Fishwild et al., Nature Biotechnology 14, 845-51, 1996; Neuberger, Nature Biotechnology 14, 826, 1996; Lonberg and Huszar, Intern. Rev. Immunol. 13:65-93, 1995; as well as U.S. Pat. Nos. 4,816,567, 5,545,807, 5,545,806, 5,569,825, 5,625,126, 5,633,425, and 5,661,016).

In some preferred embodiments, the antigen binding protein binds to a checkpoint inhibitor selected from the group consisting of PD-1, PD-L1, PD-L2, CTLA-4, CD27, CD28, CD40, CD47, CD115, CD122, CD137, OX40, GITR, ICOS, A2AR, B7-H3, B7-H4, BTLA, IDO, KIR, LAG3, NOX2, TIM-3, VISTA, SIGLEC-7, TIGIT and 4-1BB. In some embodiments, the antigen binding protein inhibits the activity of a checkpoint inhibitor selected from the group consisting of PD-1, PD-L1, PD-L2, CTLA-4, CD27, CD28, CD40, CD47, CD115, CD122, CD137, OX40, GITR, ICOS, A2AR, B7-H3, B7-H4, BTLA, IDO, KIR, LAG3, NOX2, TIM-3, VISTA, SIGLEC-7, TIGIT and 4-1BB. In some embodiments, the antibody is a commercially available human or humanized monoclonal antibody that targets VEGF (e.g., Bevacizumab (Avastin), Ranibizumab (Lucentis), or Pegaptanib (Macugen)) or an immune system checkpoint molecule such as CTLA-4 (e.g., Ipilimumab (Yervoy)), PD-L1 (e.g., Atezolizumab (Tecentriq), Avelumab (Bavencio), or Durvalumab (Imfinzi)) or PD-1 (e.g., Nivolumab (Opdivo) or Pembrolizumab (Keytruda)).

Bevacizumab (Avastin) is a recombinant humanized monoclonal antibody that blocks angiogenesis by inhibiting vascular endothelial growth factor A (VEGF-A).VEGF-A is a growth factor protein that stimulates angiogenesis in a variety of diseases, especially in cancer. Bevacizumab was the first available angiogenesis inhibitor in the United States.

Ipilimumab (Yervoy) is a monoclonal antibody that works to activate the immune system by targeting CTLA-4, a protein receptor that downregulates the immune system and is classified as checkpoint inhibitor drug. T lymphocytes can recognize and destroy cancer cells. However, an inhibitory mechanism interrupts this destruction. Ipilimumab turns off this inhibitory mechanism and allows the lymphocytes to continue to destroy cancer cells. Cancer cells produce antigens, which the immune system can use to identify them. These antigens are recognized by dendritic cells that present the antigens to cytotoxic T lymphocytes (CTLs) in the lymph nodes. The CTLs recognize the cancer cells by those antigens and destroy them. However, along with the antigens, the dendritic cells present an inhibitory signal. That signal binds to a receptor, cytotoxic T lymphocyte-associated antigen 4 (CTLA-4), on the CTL and turns off the cytotoxic reaction. This allows the cancer cells to survive. Ipilimumab binds to CTLA-4, blocking the inhibitory signal, which allows the CTLs to destroy the cancer cells.

Atezolizumab (Tecentriq) is a fully humanized, engineered monoclonal antibody of IgG1 isotype against the protein PD-L1. Avelumab (Bavencio) is a fully human monoclonal antibody that binds to PD-L1. Durvalumab (Imfinzi) is a fully human monoclonal antibody that binds to PD-L1. Together, these molecules may be referred to as PD-L1 inhibitors and are classified as immune checkpoint inhibitors. PD-L1 can be highly expressed on certain tumors, which leads to reduced activation of immune cells (cytotoxic T-cells in particular) that might otherwise recognize and attack the cancer. The PD-L1 checkpoint inhibitors block the interaction of PD-L1 with programmed cell death protein 1 (PD-1) and CD80 receptors (B7-1Rs). Inhibition of PD-L1 by removes the immune inhibitor effect and thereby engender an anti-tumor response.

Nivolumab (Opdivo) is human monoclonal antibody that binds to PD-1. Pembrolizumab (Keytruda) is a humanized antibody that binds to PD-1. Together, these molecules may be referred to as PD-1 inhibitors and are classified as immune checkpoint inhibitors. These molecules act by blocking a negative regulator of T-cell activation, thus allowing the immune system to attack the tumor. This is an example of immune checkpoint blockade. PD-1 is a protein on the surface of activated T cells. As discussed above, if PD-L1 or PD-L2 binds to PD-1, the T cell becomes inactive. Many cancer cells make PD-L1, which inhibits T cells from attacking tumors. Nivolumab blocks PD-L1 from binding to PD-1, allowing the T cell to work.

It will be understood by those of skill in the art that the CDRs or variable regions of the referenced antibodies may be isolated (e.g., by cloning) and grafted into other frameworks or antigen binding proteins derivatives (e.g., a Fab, a F(ab′)2, a Fab′ single chain antibody, a Fv single chain, bi-specific antibody, tri-specific antibody, multi-valent antibody, humanized antibody, nanobody, camelid antibody, microbody, or intrabody) as desired. This, the present invention encompasses antigen binding proteins that are derived from the reference antibodies and which are identified by the reference to the CDRs of variable regions of the reference antibodies. For example, in some preferred embodiments, the antigen binding proteins of the present invention comprise the heavy and light chain variable regions from Bevacizumab, Ranibizumab, Pegaptanib, Ipilimumab, Atezolizumab, Avelumab, Durvalumab, Nivolumab or Pembrolizumab. In other preferred embodiments, the antigen binding proteins comprise one, two or all three of CDR1, CDR2 and CDR3 from the heavy and light chain variable regions from Bevacizumab, Ranibizumab, Pegaptanib, Ipilimumab, Atezolizumab, Avelumab, Durvalumab, Nivolumab or Pembrolizumab.

### III. Multifunctional Polypeptides

Embodiments of the present invention provide multifunctional polypeptides and/or polynucleotides encoding a fusion polypeptide comprising a decorin polypeptide operably linked to a binding agent. Compositions comprising these binding agents, polypeptides, peptides, polynucleotides, expression vectors, and/or host cells are also provided in some embodiments. In certain embodiments, the compositions comprise a pharmaceutically acceptable carrier.

In some preferred embodiments, the binding agent in the multifunctional protein molecule is an antibody as described above. A diagram of a decorin-antibody fusion of the present invention that targets VEGF is provided in FIG. 1. The skilled artisan will recognize that other antigen binding proteins described herein may be substituted for the VEGF antibody described in FIG. 1. Referring to FIG. 1, the fusion protein comprises the heavy and light chains of an antibody that binds to VEGF (e.g., Bevacizumab). A decorin molecule is operably linked to the C-terminal of each heavy chain via a peptide linker. The present invention is not limited to the use of any particular peptide linker, or to linkage of the decorin molecule to any particular amino acid of the antigen binding protein. In some preferred embodiments, the decorin molecule is linked via the C-terminal of either one or two of the antibody heavy chains, via the C-terminal of either one or two of the antibody light chains, via the N-terminal of either one or two of the antibody heavy chains, via the N-terminal of either one or two of the antibody light chains, or via an amino acid in the constant region of either one or two of the antibody heavy chains that is chemically modified to allow attachment of polypeptides such as the decorin molecule. Accordingly, the fusion proteins of the present invention may comprise one or preferably two decorin molecules or functional portions thereof and may comprise more than two decorin molecules or functional portions thereof.

A number of nucleic acid constructs encoding multifunctional protein molecules of the present invention are provided in the Examples. The following chart provides a summary of the sequences.

| | |
|---|---|
| SEQ ID NO: 1 | Bevacizumab-Decorin Fusion Heavy Chain Gene |
| | Sequence with Signal Sequence (SS) |
| SEQ ID NO: 2 | Bevacizumab-Decorin Fusion Heavy Chain Protein |
| | Sequence with SS |
| SEQ ID NO: 3 | Bevacizumab Light Chain Gene Sequence with SS |
| SEQ ID NO: 4 | Bevacizumab Light Chain Protein Sequence with SS |
| SEQ ID NO: 5 | Signal sequence for heavy and light chains |
| SEQ ID NO: 6 | Linker sequence between decorin and heavy chain |
| SEQ ID NO: 7 | Decorin |
| SEQ ID NO: 8 | Bevacizumab Heavy Chain Protein Sequence |
| SEQ ID NO: 9 | Bevacizumab Light Chain Protein Sequence |
| SEQ ID NO: 10 | Ipilimumab-Galacorin Fusion Heavy Chain Gene |
| | Sequence with SS |
| SEQ ID NO: 11 | Ipilimumab-Galacorin Fusion Heavy Chain Protein |
| | Sequence with SS |
| SEQ ID NO: 12 | Ipilimumab Light Chain Gene Sequence with SS |
| SEQ ID NO: 13 | Ipilimumab Light Chain Protein Sequence with SS |
| SEQ ID NO: 14 | Ipilimumab Heavy Chain Protein Sequence |
| SEQ ID NO: 15 | Ipilimumab Light Chain Protein Sequence |
| SEQ ID NO: 16 | Atezolizumab-Galacorin Fusion Heavy Chain Gene |
| | Sequence with SS |
| SEQ ID NO: 17 | Atezolizumab-Galacorin Fusion Heavy Chain Protein |
| | Sequence with SS |
| SEQ ID NO: 18 | Atezolizumab Light Chain Gene Sequence with SS |
| SEQ ID NO: 19 | Atezolizumab Light Chain Protein Sequence with SS |
| SEQ ID NO: 20 | Atezolizumab Heavy Chain Protein Sequence |
| SEQ ID NO: 21 | Atezolizumab Light Chain Protein Sequence |
| SEQ ID NO: 22 | Avelumab-Galacorin Fusion Heavy Chain Gene |
| | Sequence with SS |
| SEQ ID NO: 23 | Avelumab-Galacorin Fusion Heavy Chain Protein |
| | Sequence with SS |
| SEQ ID NO: 24 | Avelumab Light Chain Gene Sequence with SS |
| SEQ ID NO: 25 | Avelumab Light Chain Protein Sequence with SS |
| SEQ ID NO: 26 | Avelumab Heavy Chain Protein Sequence |
| SEQ ID NO: 27 | Avelumab Light Chain Protein Sequence |
| SEQ ID NO: 28 | Durvalumab-Galacorin Fusion Heavy Chain Gene |
| | Sequence with SS |
| SEQ ID NO: 29 | Durvalumab-Galacorin Fusion Heavy Chain Protein |
| | Sequence with SS |
| SEQ ID NO: 30 | Durvalumab Light Chain Gene Sequence with SS |
| SEQ ID NO: 31 | Durvalumab Light Chain Protein Sequence with SS |
| SEQ ID NO: 32 | Durvalumab Heavy Chain Protein Sequence |
| SEQ ID NO: 33 | Durvalumab Light Chain Protein Sequence |
| SEQ ID NO: 34 | Nivolumab-Galacorin Fusion Heavy Chain Gene |
| | Sequence with SS |
| SEQ ID NO: 35 | Nivolumab-Galacorin Fusion Heavy Chain Protein |
| | Sequence with SS |
| SEQ ID NO: 36 | Nivolumab Light Chain Gene Sequence with SS |
| SEQ ID NO: 37 | Nivolumab Light Chain Protein Sequence with SS |
| SEQ ID NO: 38 | Nivolumab Heavy Chain Protein Sequence |
| SEQ ID NO: 39 | Nivolumab Light Chain Protein Sequence |
| SEQ ID NO: 40 | Pembrolizumab-Galacorin Fusion Heavy Chain Gene |
| | Sequence with SS |
| SEQ ID NO: 41 | Pembrolizumab-Galacorin Fusion Heavy Chain Protein |
| | Sequence with SS |
| SEQ ID NO: 42 | Pembrolizumab Light Chain Gene Sequence with SS |
| SEQ ID NO: 43 | Pembrolizumab Light Chain Protein Sequence with SS |
| SEQ ID NO: 44 | Pembrolizumab Heavy Chain Protein Sequence |
| SEQ ID NO: 45 | Pembrolizumab Light Chain Protein Sequence |
| SEQ ID NO: 50 | Avelumab-Galacorin Fusion Heavy Chain Gene |
| | Sequence |
| SEQ ID NO: 51 | Avelumab-Galacorin Fusion Heavy Chain Protein |
| | Sequence |
| SEQ ID NO: 52 | Avelumab Light Chain Gene Sequence |
| SEQ ID NO: 53 | Avelumab Light Chain Protein Sequence |
| SEQ ID NO: 54 | Avelumab-Galacorin2x Fusion Heavy Chain Gene |
| | Sequence |
| SEQ ID NO: 55 | Avelumab-Galacorin2x Fusion Heavy Chain Protein |
| | Sequence |
| SEQ ID NO: 56 | Avelumab-Galacorin/Decorin (Asp45-Lys359 of full |
| | length endogenous human Decorin) TGF-Beta Binding |
| | Domains Fusion Heavy Chain Gene Sequence |
| SEQ ID NO: 57 | Avelumab-Galacorin/Decorin (Asp45-Lys359 of full |
| | length endogenous human Decorin) TGF-Beta Binding |
| | Domains Fusion Heavy Chain Protein Sequence |
| SEQ ID NO: 58 | Avelumab-Galacorin/Decorin (Asp45-Lys359 of full |
| | length endogenous human Decorin) TGF-Beta Binding |
| | Domains 2x Fusion Heavy Chain Gene Sequence |
| SEQ ID NO: 59 | Avelumab-Galacorin/Decorin (Asp45-Lys359 of full |
| | length endogenous human Decorin) TGF-Beta Binding |
| | Domains 2x Fusion Heavy Chain Protein Sequence |
| SEQ ID NO: 60 | Avelumab-Galacorin/Decorin (Leu155-Val260 of full |
| | length endogenous human Decorin) TGF-Beta Binding |
| | Domain Fusion Heavy Chain Gene Sequence |
| SEQ ID NO: 61 | Avelumab-Galacorin/Decorin (Leu155-Val260 of full |
| | length endogenous human Decorin) TGF-Beta Binding |
| | Domain Fusion Heavy Chain Protein Sequence |
| SEQ ID NO: 62 | Avelumab-Galacorin/Decorin (Leu155-Val260 of full |
| | length endogenous human Decorin) TGF-Beta Binding |
| | Domain 2x Fusion Heavy Chain Gene Sequence |
| SEQ ID NO: 63 | Avelumab-Galacorin/Decorin (Leu155-Val260 of full |
| | length endogenous human Decorin) TGF-Beta Binding |
| | Domain 2x Fusion Heavy Chain Protein Sequence |

Thus, in some preferred embodiments, the fusion proteins of the present invention comprise the heavy and light chain variable regions from Bevacizumab, Ranibizumab, Pegaptanib, Ipilimumab, Atezolizumab, Avelumab, Durvalumab, Nivolumab or Pembrolizumab operably linked to a decorin molecule, preferably a decorin core protein. In other preferred embodiments, the fusion proteins comprise one, two or all three of CDR1, CDR2 and CDR3 from the heavy and light chain variable regions from Bevacizumab, Ranibizumab, Pegaptanib, Ipilimumab, Atezolizumab, Avelumab, Durvalumab, Nivolumab or Pembrolizumab operably linked to a decorin molecule, preferably a decorin core protein.

In some preferred embodiments, the decorin molecule (or molecules if more than one copy is used) is decorin core protein having at least 80%, 90%, 95%, 99% or 100% identity to SEQ ID NO:7. In other preferred embodiments, the decorin molecule (or molecules if more than one copy is used) is a decorin TGF-β binding domain having at least 80%, 90%, 95%, 99% or 100% identity to SEQ ID NO:64 or 66. In some preferred embodiments, the decorin core protein inhibits the activity of TGF-β. In some preferred embodiments, the decorin core protein comprises one or more mutations that cause the decorin core protein to be non-gagylated. In some preferred embodiments, the decorin core protein comprises a mutation at amino acid 4 (i.e., the 4^{th} amino acid from the N-terminus) of the mature decorin core protein molecule. In some preferred embodiments, the decorin core protein molecule is linked to the heavy or light chain of a target antibody via a linker sequence. The present invention is not limited to any particular linker sequence. In some preferred embodiments, the linker sequence is SEQ ID NO:6. In some particularly preferred embodiments, the linker sequence is attached to the N-terminal of the heavy chain of the antibody and position between the heavy chain and the decorin core protein. Accordingly, in some preferred embodiments, the heavy chain fusion may be represented by the following formula:
Heavy chain protein—linker—decorin core protein

In other preferred embodiments, the fusions may represented by the following formulas:
C-terminal Heavy chain protein—linker—decorin
C-terminal Light chain protein—linker—decorin
decorin—linker—N-terminal Heavy chain protein
decorin—linker—N-terminal Light chain protein
Constant region-linker-decorin
   wherein decorin may be wild-type decorin, decorin core protein, or a functional portion thereof as described in detail above and wherein the linkage is via amide bonds as in a fusion protein as is known in the art oror chemically modified amino acid in the N-terminal, C-terminal or constant region of the antigen binding protein. For example, aldehyde-tagged immunoglobulin (Ig) polypeptides can be converted by a formylglycine-generating enzyme to produce a 2-formylglycine (FGly)-modified Ig polypeptide. An FGly-modified Ig polypeptide can then be covalently and site-specifically bound to a moiety of interest to provide an Ig conjugate. See, e.g., U.S. Pat. No. 10,183,998, incorporated by reference herein in its entirety.

In some preferred embodiments, a secretion signal sequence precedes the heavy chain protein sequence to allow secretion from host cells during protein production. Likewise, in some preferred embodiments, a secretion signal sequence precedes the light chain protein sequence to allow secretion from host cells during protein production.

In some preferred embodiments, where the targeting molecule is Bevacizumab, the heavy chain sequence is at least 90%, 95%, 99% or 100% identical to SEQ ID NO:8 and the light chain sequence is at least 90%, 95%, 99% or 100% identical to SEQ ID NO:9. In some preferred embodiments, the targeting molecule binds to or inhibits VEGF-A.

In some preferred embodiments, where the targeting molecule is Ipilimumab, the heavy chain sequence is at least 90%, 95%, 99% or 100% identical to SEQ ID NO:14 and the light chain sequence is at least 90%, 95%, 99% or 100% identical to SEQ ID NO:15. In some preferred embodiments, the targeting molecule binds to or inhibits CTLA-4.

In some preferred embodiments, where the targeting molecule is Atezolizumab, the heavy chain sequence is at least 90%, 95%, 99% or 100% identical to SEQ ID NO:20 and the light chain sequence is at least 90%, 95%, 99% or 100% identical to SEQ ID NO:21. In some preferred embodiments, the targeting molecule binds to or inhibits PD-L1.

In some preferred embodiments, where the targeting molecule is Avelumab, the heavy chain sequence is at least 90%, 95%, 99% or 100% identical to SEQ ID NO:26 and the light chain sequence is at least 90%, 95%, 99% or 100% identical to SEQ ID NO:27. In some preferred embodiments, the targeting molecule binds to or inhibits PD-L1.

In some preferred embodiments, where the targeting molecule is Durvalumab, the heavy chain sequence is at least 90%, 95%, 99% or 100% identical to SEQ ID NO:32 and the light chain sequence is at least 90%, 95%, 99% or 100% identical to SEQ ID NO:33. In some preferred embodiments, the targeting molecule binds to or inhibits PD-L1.

In some preferred embodiments, where the targeting molecule is Nivolumab, the heavy chain sequence is at least 90%, 95%, 99% or 100% identical to SEQ ID NO:38 and the light chain sequence is at least 90%, 95%, 99% or 100% identical to SEQ ID NO:39. In some preferred embodiments, the targeting molecule binds to or inhibits PD-1.

In some preferred embodiments, where the targeting molecule is Pembrolizumab, the heavy chain sequence is at least 90%, 95%, 99% or 100% identical to SEQ ID NO:44 and the light chain sequence is at least 90%, 95%, 99% or 100% identical to SEQ ID NO:45. In some preferred embodiments, the targeting molecule binds to or inhibits PD-1.

In certain embodiments, preparations of fusion proteins are provided. Such preparations may comprise, for example, unpurified or purified fusion proteins. Typically, such preparations include a buffer such as phosphate- or tris-buffered saline (PBS or TBS, respectively). The preparations may also be formulated to contain excipients, like stabilizers, for example.

In some preferred embodiments, the present invention further provides nucleic acid expression constructs that encode the fusion proteins of the present invention. Accordingly, in some embodiments, the expression constructs encode the fusion protein sequences described above and are operably associated with additional nucleic acid sequences required for expression in the selected expression system.

In one example, nucleotide sequences encoding the fusion protein are constructed into a vector system, and then expressed in host cells. In one example, the host cells are cultured cells. In one example, the vector system is used in mammalian cultured cells under conditions where the fusion proteins are expressed.

The fusion polynucleotides of the present invention may be employed for producing fusion polypeptides by recombinant techniques. Thus, for example, the polynucleotide may be included in any one of a variety of expression vectors for expressing a polypeptide. In some embodiments of the present invention, vectors include, but are not limited to, retroviral vectors, chromosomal, nonchromosomal and synthetic DNA sequences (e.g., derivatives of SV40, bacterial plasmids, phage DNA; baculovirus, yeast plasmids, vectors derived from combinations of plasmids and phage DNA, and viral DNA such as vaccinia, adenovirus, fowl pox virus, and pseudorabies). It is contemplated that any vector may be used as long as it is replicable and viable in the host. In some preferred embodiments, the vectors are retroviral vectors as described in U.S. Pat. Nos. 6,852,510 and 7,332,333 and U.S. pat. Publ. Nos. 200402335173 and 20030224415, all of which are incorporated herein by references in their entirety. In some especially preferred embodiments, the vectors are pseudotyped retroviral vectors.

In particular, some embodiments of the present invention provide recombinant constructs comprising one or more of the sequences as broadly described above. In some embodiments of the present invention, the constructs comprise a vector, such as a plasmid or viral vector, into which a sequence of the invention has been inserted, in a forward or reverse orientation. In still other embodiments, the heterologous structural sequence is assembled in appropriate phase with translation initiation and termination sequences. In preferred embodiments of the present invention, the appropriate DNA sequence is inserted into the vector using any of a variety of procedures. In general, the DNA sequence is inserted into an appropriate restriction endonuclease site(s) by procedures known in the art.

Large numbers of suitable vectors are known to those of skill in the art, and are commercially available. Such vectors include, but are not limited to, the following vectors: 1) Bacterial—pQE70, pQE60, pQE-9 (Qiagen), pBS, pD10, phagescript, psiX174, pbluescript SK, pBSKS, pNH8A, pNH16a, pNH18A, pNH46A (Stratagene); ptrc99a, pKK223-3, pKK233-3, pDR540, pRIT5 (Pharmacia); 2) Eukaryotic—pWLNEO, pSV2CAT, pOG44, PXT1, pSG (Stratagene) pSVK3, pBPV, pMSG, pSVL (Pharmacia); and 3) Baculovirus—pPbac and pMbac (Stratagene). Any other plasmid or vector may be used as long as they are replicable and viable in the host. In some preferred embodiments of the present invention, mammalian expression vectors comprise an origin of replication, a suitable promoter and enhancer, and also any necessary ribosome binding sites, polyadenylation sites, splice donor and acceptor sites, transcriptional termination sequences, and 5′ flanking non-transcribed sequences. In other embodiments, DNA sequences derived from the SV40 splice, and polyadenylation sites may be used to provide the required non-transcribed genetic elements.

In certain embodiments of the present invention, the DNA sequence in the expression vector is operatively linked to an appropriate expression control sequence(s) (promoter) to direct mRNA synthesis. Promoters useful in the present invention include, but are not limited to, the LTR or SV40 promoter, the E. coli lac or trp, the phage lambda P_{L} and P_{R}, T3 and T7 promoters, and the cytomegalovirus (CMV) immediate early, herpes simplex virus (HSV) thymidine kinase, and mouse metallothionein-I promoters and other promoters known to control expression of gene in prokaryotic or eukaryotic cells or their viruses. In other embodiments of the present invention, recombinant expression vectors include origins of replication and selectable markers permitting transformation of the host cell (e.g., dihydrofolate reductase or neomycin resistance for eukaryotic cell culture, or tetracycline or ampicillin resistance in E. coli).

In some embodiments of the present invention, transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Enhancers useful in the present invention include, but are not limited to, the SV40 enhancer on the late side of the replication origin bp 100 to 270, a cytomegalovirus early promoter enhancer, the polyoma enhancer on the late side of the replication origin, and adenovirus enhancers.

In other embodiments, the expression vector also contains a ribosome binding site for translation initiation and a transcription terminator. In still other embodiments of the present invention, the vector may also include appropriate sequences for amplifying expression.

In a further embodiment, the present invention provides host cells containing the above-described constructs. In some embodiments of the present invention, the host cell is a higher eukaryotic cell (e.g., a mammalian or insect cell). In other embodiments of the present invention, the host cell is a lower eukaryotic cell (e.g., a yeast cell). In still other embodiments of the present invention, the host cell can be a prokaryotic cell (e.g., a bacterial cell). Specific examples of host cells include, but are not limited to, Escherichia coli, Salmonella typhimurium, Bacillus subtilis, and various species within the genera Pseudomonas, Streptomyces, and Staphylococcus, as well as Saccharomycees cerivisiae, Schizosaccharomycees pombe, Drosophila S2 cells, Spodoptera Sf9 cells, Chinese hamster ovary (CHO) cells, COS-7 lines of monkey kidney fibroblasts, (Gluzman, Cell 23:175 [1981]), C127, 3T3, 293, 293T, HeLa and BHK cell lines.

The constructs in host cells can be used in a conventional manner to produce the gene product encoded by the recombinant sequence. In some embodiments, introduction of the construct into the host cell can be accomplished by retroviral transduction, calcium phosphate transfection, DEAE-Dextran mediated transfection, or electroporation (See e.g., Davis et al. [1986] Basic Methods in Molecular Biology). Alternatively, in some embodiments of the present invention, the polypeptides of the invention can be synthetically produced by conventional peptide synthesizers.

Proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al. (1989) Molecular Cloning: A Laboratory Manual, Second Edition, Cold Spring Harbor, N.Y.

In some embodiments of the present invention, following transformation of a suitable host strain and growth of the host strain to an appropriate cell density in media, protein is secreted and cells are cultured for an additional period. In other embodiments of the present invention, cells are typically harvested by centrifugation, disrupted by physical or chemical means, and the resulting crude extract retained for further purification. In still other embodiments of the present invention, microbial cells employed in expression of proteins can be disrupted by any convenient method, including freeze-thaw cycling, sonication, mechanical disruption, or use of cell lysing agents.

Further embodiments provide kits including fusion proteins and optionally other components useful, necessary, or sufficient for using the fusion proteins (e.g., therapeutic, research, and screening applications). The fusion proteins of the kit may be provided in any suitable form, including frozen, lyophilized, or in a pharmaceutically acceptable buffer such as TBS or PBS.

The fusion proteins described herein and/or derivatives thereof may also be incorporated into compositions for use in vitro or in vivo. The antibody, fusion protein, or derivatives thereof may also be fixably attached to functional effector moieties such as cytotoxic drugs or toxins, or active fragments thereof such as diphtheria A chain, exotoxin A chain, ricin A chain, abrin A chain, curcin, crotin, phenomycin, enomycin, among others.

Functional moieties may also include radiochemicals. In one embodiment, the effector moieties may be fixably attached to the binding agents. In one example, the detectable labels are fixably attached to the binding agents by chemical bonds. In one example, the chemical bonds are covalent chemical bonds. In one example, the effector moieties are conjugated to the binding agents.

The fusion proteins described herein may be prepared as an injectable preparation, such as in suspension in a non-toxic parenterally acceptable diluent or solvent. Suitable vehicles and solvents that may be utilized include water, Ringer's solution, and isotonic sodium chloride solution, TBS and PBS, among others. The formulations may contain excipients, like stablizers, for example. In certain applications, the antibodies are suitable for use in vitro. In other applications, the antibodies are suitable for use in vivo. The preparations suitable for use in either case are well-known in the art and will vary depending on the particular application.

The fusion proteins may be combined with one or more pharmaceutically acceptable carriers prior to administration to a host. A pharmaceutically acceptable carrier is a material that is not biologically or otherwise undesirable, e.g., the material may be administered to a subject, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. Suitable pharmaceutical carriers and their formulations are described in, for example, Remington s: The Science and Practice of Pharmacy, 2 yt Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose. The pH of the solution is generally from about 5 to about 8 or from about 7 to about 7.5. Other carriers include sustained-release preparations such as semipermeable matrices of solid hydrophobic polymers containing polypeptides or fragments thereof. Matrices may be in the form of shaped articles, e.g., films, liposomes or microparticles. It will be apparent to those persons skilled in the art that certain carriers may be more preferable depending upon, for instance, the route of administration and concentration of composition being administered. Carriers are those suitable for administration of polypeptides and/or fragments thereof to humans or other subjects.

Pharmaceutical compositions may also include carriers, thickeners, diluents, buffers, preservatives, surface active agents, adjuvants, immunostimulants, in addition to the fusion protein. Pharmaceutical compositions may also include one or more active ingredients such as antimicrobial agents, anti-inflammatory agents and anesthetics

### IV. Uses

Embodiments of the present disclosure provide compositions and methods for research, screening, and therapeutic application. For example, embodiments of the present invention provide methods of treating a variety of diseases using the multifunctional protein molecules described herein.

In some embodiments, the compositions and methods of the present invention are used to treat diseased cells, tissues, organs, or pathological conditions and/or disease states in an animal (e.g., a mammalian patient including, but not limited to, humans and veterinary animals). In this regard, various diseases and pathologies are amenable to treatment or prophylaxis using the present methods and compositions. A non-limiting exemplary list of these diseases and conditions includes, but is not limited to, pancreatic cancer, breast cancer, prostate cancer, lymphoma, skin cancer, colon cancer, melanoma, malignant melanoma, ovarian cancer, brain cancer, primary brain carcinoma, head—neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like, and macular degeneration.

Some embodiments of the present invention provide methods for administering an effective amount of a fusion polypeptide of the invention and at least one additional therapeutic agent (including, but not limited to, chemotherapeutic antineoplastics, apoptosis-modulating agents, antimicrobials, antivirals, antifungals, and anti-inflammatory agents) and/or therapeutic technique (e.g., surgical intervention, and/or radiotherapies). In a particular embodiment, the additional therapeutic agent(s) is an anticancer agent.

A number of suitable anticancer agents are contemplated for use in the methods of the present invention. Indeed, the present invention contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of chemotherapeutic compounds and anticancer therapies suitable for co-administration with the disclosed compounds are known to those skilled in the art.

In certain embodiments, anticancer agents comprise agents that induce or stimulate apoptosis. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present invention provide a compound of the invention and at least one anti-hyperproliferative or antineoplastic agent selected from alkylating agents, antimetabolites, and natural products (e.g., herbs and other plant and/or animal derived compounds).

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the compositions and methods of the present invention include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′— DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. The Table below provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

| | | |
|---|---|---|
| Aldesleukin | Proleukin | Chiron Corp., Emeryville, |
| (des-alanyl-1, serine-125 human | | CA |
| interleukin-2) | | |
| Alemtuzumab | Campath | Millennium and ILEX |
| (IgG1κ anti CD52 antibody) | | Partners, LP, Cambridge, |
| | | MA |
| Alitretinoin | Panretin | Ligand Pharmaceuticals, |
| (9-cis-retinoic acid) | | Inc., San Diego CA |
| Allopurinol | Zyloprim | GlaxoSmithKline, Research |
| (1,5-dihydro-4H-pyrazolo[3,4- | | Triangle Park, NC |
| d]pyrimidin-4-one monosodium salt) | | |
| Altretamine | Hexalen | US Bioscience, West |
| (N,N,N′,N′,N″,N″,-hexamethyl-1,3,5- | | Conshohocken, PA |
| triazine-2,4,6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile, a,a,a′,a′- | | Pharmaceuticals, LP, |
| tetramethyl-5-(1H-1,2,4-triazol-1- | | Wilmington, DE |
| ylmethyl)) | | |
| Arsenic trioxide | Trisenox | Cell Therapeutic, Inc., |
| | | Seattle, WA |
| Asparaginase | Elspar | Merck & Co Inc |
| (L-asparagine amidohydrolase, type EC-2) | | Whitehouse Station, NJ |
| BCG Live | TICE BCG | Organon Teknika, Corp., |
| (lyophilized preparation of an attenuated | | Durham, NC |
| strain of Mycobacterium bovis (Bacillus | | |
| Calmette-Gukin [BCG], substrain Montreal) | | |
| bexarotene capsules | Targretin | Ligand Pharmaceuticals |
| (4-[1-(5,6,7,8-tetrahydro-3,5,5,8,8- | | |
| pentamethyl-2-napthalenyl) ethenyl] | | |
| benzoic acid) | | |
| bexarotene gel | Targretin | Ligand Pharmaceuticals |
| Bleomycin | Blenoxane | Bristol-Myers Squibb Co., |
| (cytotoxic glycopeptide antibiotics | | NY, NY |
| produced by Streptomyces verticillus; | | |
| bleomycin A₂and bleomycin B₂) | | |
| Capecitabine | Xeloda | Roche |
| (5′-deoxy-5-fluoro-N- | | |
| [(pentyloxy)carbonyl]-cytidine) | | |
| Carboplatin | Paraplatin | Bristol-Myers Squibb |
| (platinum, diammine [1,1- | | |
| cyclobutanedicarboxylato(2-)-0,0′]-,(SP-4- | | |
| 2)) | | |
| Carmustine | BCNU, | Bristol-Myers Squibb |
| (1,3-bis(2-chloroethyl)-1-nitrosourea) | BiCNU | |
| Carmustine with Polifeprosan 20 Implant | Gliadel | Guilford Pharmaceuticals, |
| | Wafer | Inc., Baltimore, MD |
| Celecoxib | Celebrex | Searle Pharmaceuticals, |
| (as 4-[5-(4-methylphenyl)-3- | | England |
| (trifluoromethyl)-1H-pyrazol-1-yl] | | |
| benzenesulfonamide) | | |
| Chlorambucil | Leukeran | GlaxoSmithKline |
| (4- | | |
| [bis(2chlorethyl)amino]benzenebutanoic | | |
| acid) | | |
| Cisplatin | Platinol | Bristol-Myers Squibb |
| (PtCl₂H₆N₂) | | |
| Cladribine | Leustatin, | R.W. Johnson |
| (2-chloro-2′-deoxy-b-D-adenosine) | 2-CdA | Pharmaceutical Research |
| | | Institute, Raritan, NJ |
| Cyclophosphamide | Cytoxan, | Bristol-Myers Squibb |
| (2-[bis(2-chloroethyl)amino] tetrahydro- | Neosar | |
| 2H-13,2-oxazaphosphorine 2-oxide | | |
| monohydrate) | | |
| Cytarabine | Cytosar-U | Pharmacia & Upjohn |
| (1-b-D-Arabinofuranosylcytosine, | | Company |
| C₉H₁₃N₃O₅) | | |
| cytarabine liposomal | DepoCyt | Skye Pharmaceuticals, Inc., |
| | | San Diego, CA |
| Dacarbazine | DTIC- | Bayer AG, Leverkusen, |
| (5-(3,3-dimethyl-l-triazeno)-imidazole-4- | Dome | Germany |
| carboxamide (DTIC)) | | |
| Dactinomycin, actinomycin D | Cosmegen | Merck |
| (actinomycin produced by Streptomyces | | |
| parvullus, C₆₂H₈₆N₁₂O₁₆) | | |
| Darbepoetin alfa | Aranesp | Amgen, Inc., Thousand |
| (recombinant peptide) | | Oaks, CA |
| daunorubicin liposomal | DanuoXome | Nexstar Pharmaceuticals, |
| ((8S-cis)-8-acetyl-10-[(3-amino-2,3,6- | | Inc., Boulder, CO |
| trideoxy-á-L-lyxo-hexopyranosyl)oxy]- | | |
| 7,8,9,10-tetrahydro-6,8,11-trihydroxy-1- | | |
| methoxy-5,12-naphthacenedione | | |
| hydrochloride) | | |
| Daunorubicin HCl, daunomycin | Cerubidine | Wyeth Ayerst, Madison, |
| ((1S,3S)-3-Acetyl-1,2,3,4,6,11- | | NJ |
| hexahydro-3,5,12-trihydroxy-10-methoxy- | | |
| 6,11-dioxo-1-naphthacenyl 3-amino-2,3,6- | | |
| trideoxy-(alpha)-L-lyxo-hexopyranoside | | |
| hydrochloride) | | |
| Denileukin diftitox | Ontak | Seragen, Inc., Hopkinton, |
| (recombinant peptide) | | MA |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis Pharmaceuticals, |
| ((2R,3S)-N-carboxy-3-phenylisoserine, N- | | Inc., Bridgewater, NJ |
| tert-butyl ester, 13-ester with 5b-20- | | |
| epoxy-12a,4,7b,10b,13a-hexahydroxytax- | | |
| 11-en-9-one 4-acetate 2-benzoate, | | |
| trihydrate) | | |
| Doxorubicin HCl | Adriamycin, | Pharmacia & Upjohn |
| (8S,10S)-10-[(3-amino-2,3,6-trideoxy-a-L- | Rubex | Company |
| lyxo-hexopyranosyl)oxy]-8-glycolyl- | | |
| 7,8,9,10-tetrahydro-6,8,11-trihydroxy-1- | | |
| methoxy-5,12-naphthacenedione | | |
| hydrochloride) | | |
| doxorubicin | Adriamycin | Pharmacia & Upjohn |
| | PFS | Company |
| | Intravenous | |
| | injection | |
| doxorubicin liposomal | Doxil | Sequus Pharmaceuticals, |
| | | Inc., Menlo park, CA |
| dromostanolone propionate | Dromostanolone | Eli Lilly & Company, |
| (17b-Hydroxy-2a-methyl-5a-androstan-3- | | Indianapolis, IN |
| one propionate) | | |
| dromostanolone propionate | Masterone | Syntex, Corp., Palo Alto, |
| | injection | CA |
| Elliott's B Solution | Elliott's B | Orphan Medical, Inc |
| | Solution | |
| Epirubicin | Ellence | Pharmacia & Upjohn |
| ((8S-cis)-10-[(3-amino-2,3,6-trideoxy-a-L- | | Company |
| arabino-hexopyranosyl)oxy]-7,8,9,10- | | |
| tetrahydro-6,8,11-trihydroxy-8- | | |
| (hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, | | Company |
| 3-[bis(2-chloroethyl)carbamate] 17- | | |
| (dihydrogen phosphate), disodium salt, | | |
| monohydrate, or estradiol 3-[bis(2- | | |
| chloroethyl)carbamate] 17-(dihydrogen | | |
| phosphate), disodium salt, monohydrate) | | |
| Etoposide phosphate | Etopophos | Bristol-Myers Squibb |
| (4′-Demethylepipodophyllotoxin 9-[4,6-O- | | |
| (R)-ethylidene-(beta)-D-glucopyranoside], | | |
| 4′-(dihydrogen phosphate)) | | |
| etoposide, VP-16 | Vepesid | Bristol-Myers Squibb |
| (4′-demethylepipodophyllotoxin 9-[4,6-0- | | |
| (R)-ethylidene-(beta)-D-glucopyranoside]) | | |
| Exemestane | Aromasin | Pharmacia & Upjohn |
| (6-methylenandrosta-1,4-diene-3,17- | | Company |
| dione) | | |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, Inc., |
| (fluorinated nucleotide analog of the | | Cedar Knolls, NJ |
| antiviral agent vidarabine, 9-b-D- | | |
| arabinofuranosyladenine (ara-A)) | | |
| Fluorouracil, 5-FU | Adrucil | ICN Pharmaceuticals, Inc., |
| (5-fluoro-2,4(1H,3H)-pyrimidinedione) | | Humacao, Puerto Rico |
| Fulvestrant | Faslodex | IPR Pharmaceuticals, |
| (7-alpha-[9-(4,4,5,5,5-penta | | Guayama, Puerto Rico |
| fluoropentylsulphinyl) nonyl]estra-1,3,5- | | |
| (10)-triene-3,17-beta-diol) | | |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex | AstraZeneca |
| | Implant | Pharmaceuticals |
| Hydroxyurea | Hydrea | Bristol-Myers Squibb |
| Ibritumomab Tiuxetan | Zevalin | Biogen IDEC, Inc., |
| (immunoconjugate resulting from a | | Cambridge MA |
| thiourea covalent bond between the | | |
| monoclonal antibody Ibritumomab and | | |
| the linker-chelator tiuxetan [N-[2- | | |
| bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & Upjohn |
| (5,12-Naphthacenedione, 9-acetyl-7-[(3- | | Company |
| amino-2,3,6-trideoxy-(alpha)-L-lyxo- | | |
| hexopyranosyl)oxy]-7,8,9,10-tetrahydro- | | |
| 6,9,11-trihydroxyhydrochloride, (7S-cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4- | | Switzerland |
| methyl-3-[[4-(3-pyridinyl)-2- | | |
| pyrimidinyl]amino]-phenyl]benzamide | | |
| methanesulfonate) | | |
| Interferon alfa-2a | Roferon-A | Hoffmann-La Roche, Inc., |
| (recombinant peptide) | | Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| ((4S)-4,11-diethyl-4-hydroxy-9-[(4-piperi- | | Company |
| dinopiperidino)carbonyloxy]-1H-pyrano[3′, | | |
| 4′:6,7] indolizino[1,2-b] quinoline- | | |
| 3,14(4H,12H) dione hydrochloride | | |
| trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., Seattle, |
| (L-Glutamic acid, N[4[[(2amino-5-formyl- | Leucovorin | WA |
| 1,4,5,6,7,8 hexahydro4oxo6- | | |
| pteridinyl)methyl]amino]benzoyl], calcium | | |
| salt (1:1)) | | |
| Levamisole HCl | Ergamisol | Janssen Research |
| ((−)-(S)-2,3,5,6-tetrahydro-6- | | Foundation, Titusville, NJ |
| phenylimidazo [2,1-b] thiazole | | |
| monohydrochloride C₁₁H₁₂N₂S•HCl) | | |
| Lomustine | CeeNU | Bristol-Myers Squibb |
| (1-(2-chloro-ethyl)-3-cyclohexyl-1- | | |
| nitrosourea) | | |
| Meclorethamine, nitrogen mustard | Mustargen | Merck |
| (2-chloro-N-(2-chloroethyl)-N- | | |
| methylethanamine hydrochloride) | | |
| Megestrol acetate | Megace | Bristol-Myers Squibb |
| 17α(acetyloxy)-6-methylpregna-4,6- | | |
| diene-3,20-dione | | |
| Melphalan, L-PAM | Alkeran | GlaxoSmithKline |
| (4-[bis(2-chloroethyl) amino]-L- | | |
| phenylalanine) | | |
| Mercaptopurine, 6-MP | Purinethol | GlaxoSmithKline |
| (1,7-dihydro-6H-purine-6-thione | | |
| monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]- | | |
| L-glutamic acid) | | |
| Methoxsalen | Uvadex | Therakos, Inc., Way Exton, |
| (9-methoxy-7H-furo[3,2-g][1]-benzopyran- | | Pa |
| 7-one) | | |
| Mitomycin C | Mutamycin | Bristol-Myers Squibb |
| mitomycin C | Mitozytrex | SuperGen, Inc., Dublin, CA |
| Mitotane | Lysodren | Bristol-Myers Squibb |
| (1,1-dichloro-2-(o-chlorophenyl)-2-(p- | | |
| chlorophenyl) ethane) | | |
| Mitoxantrone | Novantrone | Immunex Corporation |
| (1,4-dihydroxy-5,8-bis[[2-[(2- | | |
| hydroxyethyl)amino]ethyl]amino]-9,10- | | |
| anthracenedione dihydrochloride) | | |
| Nandrolone phenpropionate | Durabolin- | Organon, Inc., West |
| | 50 | Orange, NJ |
| Nofetumomab | Verluma | Boehringer Ingelheim |
| | | Pharma KG, Germany |
| Oprelvekin | Neumega | Genetics Institute, Inc., |
| (IL-11) | | Alexandria, VA |
| Oxaliplatin | Eloxatin | Sanofi Synthelabo, Inc., |
| (cis-[(1R,2R)-1,2-cyclohexanediamine- | | NY, NY |
| N,N′] [oxalato(2-)-O,O′] platinum) | | |
| Paclitaxel | TAXOL | Bristol-Myers Squibb |
| (5β,20-Epoxy-1,2a,4,7β,10β,13a- | | |
| hexahydroxytax-11-en-9-one 4,10- | | |
| diacetate 2-benzoate 13-ester with (2R,3S)-N- | | |
| benzoyl-3-phenylisoserine) | | |
| Pamidronate | Aredia | Novartis |
| (phosphonic acid (3-amino-1- | | |
| hydroxypropylidene) bis-, disodium salt, | | |
| pentahydrate, (APD)) | | |
| Pegademase | Adagen | Enzon Pharmaceuticals, |
| ((monomethoxypolyethylene glycol | (Pegademase | Inc., Bridgewater, NJ |
| succinimidyl) 11-17-adenosine | Bovine) | |
| deaminase) | | |
| Pegaspargase | Oncaspar | Enzon |
| (monomethoxypolyethylene glycol | | |
| succinimidyl L-asparaginase) | | |
| Pegfilgrastim | Neulasta | Amgen, Inc |
| (covalent conjugate of recombinant | | |
| methionyl human G-CSF (Filgrastim) and | | |
| monomethoxypolyethylene glycol) | | |
| Pentostatin | Nipent | Parke-Davis |
| | | Pharmaceutical Co., |
| | | Rockville, MD |
| Pipobroman | Vercyte | Abbott Laboratories, |
| | | Abbott Park, IL |
| Plicamycin, Mithramycin | Mithracin | Pfizer, Inc., NY, NY |
| (antibiotic produced by Streptomyces | | |
| plicatus) | | |
| Porfimer sodium | Photofrin | QLT Phototherapeutics, |
| | | Inc., Vancouver, |
| | | Canada |
| Procarbazine | Matulane | Sigma Tau |
| (N-isopropyl-μ-(2-methylhydrazino)-p- | | Pharmaceuticals, Inc., |
| toluamide monohydrochloride) | | Gaithersburg, MD |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi-Synthelabo, Inc., |
| (recombinant peptide) | | |
| Rituximab | Rituxan | Genentech, Inc., South San |
| (recombinant anti-CD20 antibody) | | Francisco, CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]- | | |
| a(and b)-D-glucopyranose and 220 mg | | |
| citric acid anhydrous) | | |
| Talc | Sclerosol | Bryan, Corp., Woburn, MA |
| (Mg₃Si₄O₁₀(OH)₂) | | |
| Tamoxifen | Nolvadex | AstraZeneca |
| ((Z)2-[4-(1,2-diphenyl-1-butenyl) | | Pharmaceuticals |
| phenoxy]-N,N-dimethylethanamine 2- | | |
| hydroxy-1,2,3-propanetricarboxylate | | |
| (1:1)) | | |
| Temozolomide | Temodar | Schering |
| (3,4-dihydro-3-methyl-4-oxoimidazo]5,1- | | |
| d]-as-tetrazine-8-carboxamide) | | |
| teniposide, VM-26 | Vumon | Bristol-Myers Squibb |
| (4′-demethylepipodophyllotoxin 9-[4,6-0- | | |
| (R)-2-thenylidene-(beta)-D- | | |
| glucopyranoside]) | | |
| Testolactone | Teslac | Bristol-Myers Squibb |
| (13-hydroxy-3-oxo-13,17-secoandrosta- | | |
| 1,4-dien-17-oic acid [dgr]-lactone) | | |
| Thioguanine, 6-TG | Thioguanine | GlaxoSmithKline |
| (2-amino-1,7-dihydro-6 H-purine-6- | | |
| thione) | | |
| Thiotepa | Thioplex | Immunex Corporation |
| (Aziridine, 1,1′,1″- | | |
| phosphinothioylidynetris-, or Tris (1- | | |
| aziridinyl) phosphine sulfide) | | |
| Topotecan HCl | Hycamtin | GlaxoSmithKline |
| ((S)-10-[(dimethylamino) methyl]-4-ethyl- | | |
| 4,9-dihydroxy-1H-pyrano[3′,4′:6,7] | | |
| indolizino[1,2-b] quinoline-3,14-(4H,12H)- | | |
| dione monohydrochloride) | | |
| Toremifene | Fareston | Roberts Pharmaceutical |
| (2-(p-[(Z)-4-chloro-1,2-diphenyl-1- | | Corp., Eatontown, NJ |
| butenyl]-phenoxy)-N,N- | | |
| dimethylethylamine citrate (1:1)) | | |
| Tositumomab, I 131 Tositumomab | Bexxar | Corixa Corp., Seattle, WA |
| (recombinant murine immunotherapeutic | | |
| monoclonal IgG₂ₐ lambda anti-CD20 | | |
| antibody (I 131 is a | | |
| radioimmunotherapeutic antibody)) | | |
| Trastuzumab | Herceptin | Genentech, Inc |
| (recombinant monoclonal IgG₁kappa anti- | | |
| HER2 antibody) | | |
| Tretinoin, ATRA | Vesanoid | Roche |
| (all-trans retinoic acid) | | |
| Uracil Mustard | Uracil | Roberts Labs |
| | Mustard | |
| | Capsules | |
| Valrubicin, N-trifluoroacetyladriamycin-14- | Valstar | Anthra --> Medeva |
| valerate | | |
| ((2S-cis)-2-[1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L- | | |
| lyxo-hexopyranosyl]oxyl]-2-naphthacenyl]- | | |
| 2-oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′- | | |
| norvincaleukoblastine [R-(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl- | | |
| phosphonoethyl) phosphonic acid | | |
| monohydrate) | | |

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, S-1, S-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

In some embodiments of the present invention, a fusion protein of the invention and one or more therapeutic agents or anticancer agents are administered to an animal under one or more of the following conditions: at different periodicities, at different durations, at different concentrations, by different administration routes, etc.

Fusion proteins within the scope of this invention include all fusion proteins described herein wherein the fusion proteins of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than Cu). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXPERIMENTAL

### Example 1

### Expression Constructs

This example describes the design of the expression constructs that are inserted into expression vectors for recombinant production of fusion proteins.

### Anti-VEGF

Bevacizumab-Galacorin Fusion Heavy Chain Gene Sequence (SEQ ID NO: 1):
ATGATGTCCTTTGTCTCTCTGCTCCTGGTTGGCATCCTATTCCATGCCACCCAGGCCGAGGTGC
AGCTGGTGGAGTCTGGGGGAGGCTTGGTACAGCCTGGGGGGTCCCTGAGACTCTCCTGTGCAGC
CTCTGGATACACCTTTACCAACTATGGCATGAACTGGGTCCGCCAGGCTCCAGGGAAGGGGCTG
GAGTGGGTGGGCTGGATAAACACTTACACTGGTGAGCCAACATATGCAGCTGACTTCAAGCGCC
GGTTTACCTTCTCTTTGGACACCTCCAAGTCCACGGCCTATCTGCAAATGAACAGCCTGCGGGC
CGAGGACACGGCCGTATATTACTGTGCGAAATACCCCCACTACTACGGTAGTAGCCACTGGTAC
TTTGACGTGTGGGGCCAGGGAACCCTGGTCACCGTCTCCTCAGCCTCCACCAAGGGCCCATCGG
TCTTCCCCCTGGCACCCTCCTCCAAGAGCACCTCTGGGGGCACAGCGGCCCTGGGCTGCCTGGT
CAAGGACTACTTCCCCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAGCGTGGTGACCGTGC
CCTCCAGCAGCTTGGGCACCCAGACCTACATCTGCAACGTGAATCACAAGCCCAGCAACACCAA
GGTGGACAAGAAAGTTGAGCCCAAATCTTGTGACAAAACTCACACATGCCCACCGTGCCCAGCA
CCTGAACTCCTGGGGGGACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGAGGTCAA
GTTCAACTGGTACGTGGACGGCGTGGAGGTGCATAATGCCAAGACAAAGCCGCGGGAGGAGCAG
TACAACAGCACGTACCGGGTGGTCAGCGTCCTCACCGTCCTGCACCAGGACTGGCTGAATGGCA
AGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGT
GGGAGAGCAATGGGCAGCCGGAGAACAACTACAAGACCACGCCTCCCGTGCTGGACTCCGACGG
CTCCTTCTTCCTCTACAGCAAGCTCACCGTGGACAAGAGCAGGTGGCAGCAGGGGAACGTCTTC
TCATGCTCCGTGATGCATGAGGCTCTGCACAACCACTACACGCAGAAGAGCCTCTCCCTGTCTC

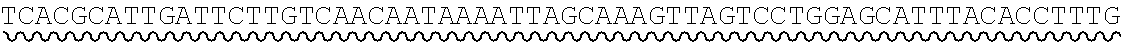

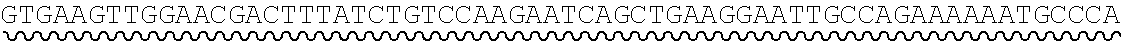

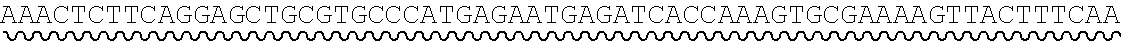

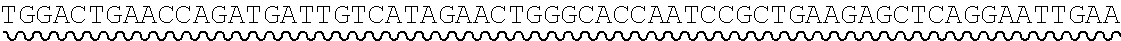

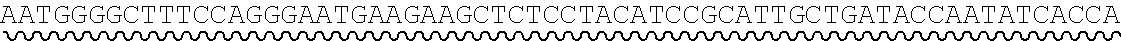

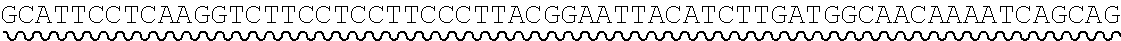

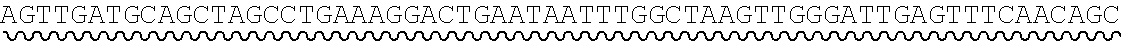

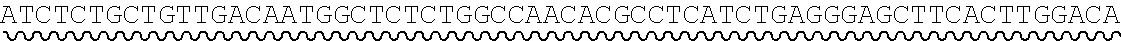

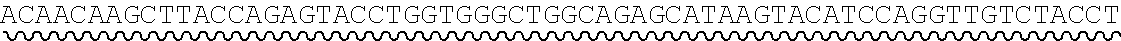

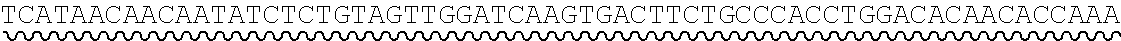

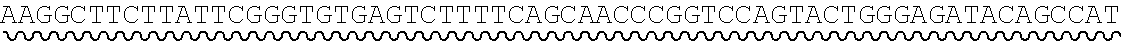

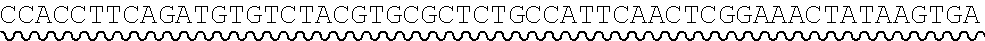

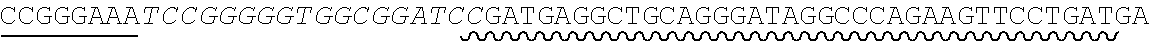

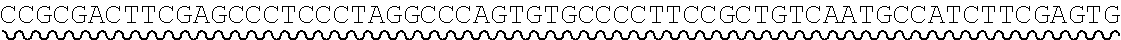

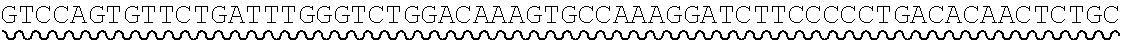

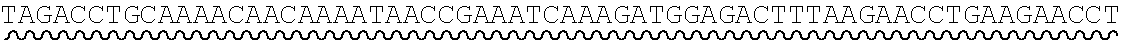

Signal peptide coding sequence is shown in boldface type. Heavy chain sequence is underlined with a straight line. Linker coding sequence is shown in italics. Galacorin coding sequence is underlined with a wavy line.

Bevacizumab-Galacorin Fusion Heavy Chain Protein Sequence (SEQ ID NO: 2):
MMSFVSLLLVGILFHATQAEVQLVESGGGLVQPGGSLRLSCAASGYTFTNYGMNWVRQAPGKGL
EWVGWINTYTGEPTYAADFKRRFTFSLDTSKSTAYLQMNSLRAEDTAVYYCAKYPHYYGSSHWY
FDVWGQGTLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMT
KNQVSLTCLVKGFYPSDIAVEWESNGQPENNYKTTPPVLDSDGSFFLYSKLTVDKSRWQQGNVF

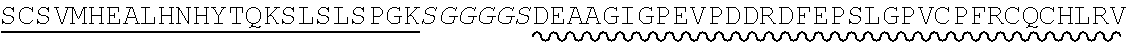

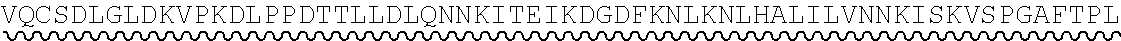

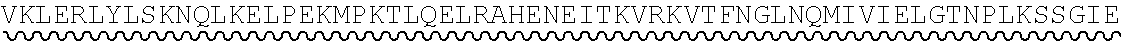

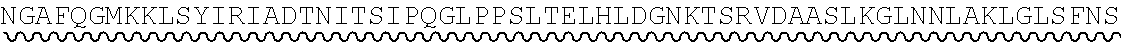

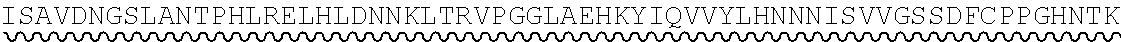

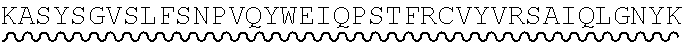

Signal peptide sequence is shown in boldface type. Heavy chain sequence is underlined with a straight line. Linker sequence is shown in italics. Galacorin sequence is underlined with a wavy line.

Bevacizumab Light Chain Gene Sequence
(SEQ ID NO: 3):
ATGATGTCCTTTGTCTCTCTGCTCCTGGTTGGCATCCTGTTCCATGCCACC
CAGGCCGACATCCAGATGACCCAGTCTCCATCCTCCCTGTCTGCATCTGTA
GGAGACAGAGTCACCATCACTTGCAGTGCAAGTCAGGACATTAGCAATTAT
TTAAACTGGTATCAGCAGAAACCAGGGAAAGCTCCTAAGGTCCTGATCTAT
TTCACATCCAGTTTGCACTCAGGGGTCCCATCTAGGTTCAGTGGCAGTGGA
TCTGGGACAGATTTCACTCTCACCATCAGCAGCCTGCAGCCTGAAGATTTT
GCAACTTATTACTGCCAACAGTATAGTACCGTGCCTTGGACGTTCGGCCAA
GGGACCAAGGTGGAAATCAAACGAACTGTGGCTGCACCATCTGTCTTCATC
TTCCCGCCATCTGATGAGCAGCTTAAGTCCGGAACTGCTAGCGTTGTGTGC
CTGCTGAATAACTTCTATCCCAGAGAGGCCAAAGTACAGTGGAAGGTGGAT
AACGCCCTCCAATCGGGTAACTCCCAGGAGAGTGTCACAGAGCAGGACAGC
AAGGACAGCACCTACAGCCTCAGCAGCACCCTGACGCTGAGCAAAGCAGAC
TACGAGAAACACAAAGTCTACGCCTGCGAAGTCACCCATCAGGGCCTGAGC
TCGCCCGTCACAAAGAGCTTCAACAGGGGAGAGTGTTAG

Signal peptide coding sequence is shown in boldface type. Light chain coding sequence is underlined with a straight line.

Bevacizumab Light Chain Protein Sequence
(SEQ ID NO: 4):
MMSFVSLLLVGILFHATQADIQMTQSPSSLSASVGDRVTITCSASQDISNY
LNWYQQKPGKAPKVLIYFTSSLHSGVPSRFSGSGSGTDFTLTISSLQPEDF
ATYYCQQYSTVPWTFGQGTKVEIKRTVAAPSVFIFPPSDEQLKSGTASVVC
LLNNFYPREAKVQWKVDNALQSGNSQESVTEQDSKDSTYSLSSTLTLSKAD
YEKHKVYACEVTHQGLSSPVTKSFNRGEC

Signal peptide sequence is shown in boldface type. Light chain sequence is underlined with a straight line.

Signal sequence for heavy and light chains
(SEQ ID NO: 5):
MMSFVSLLLVGILFHATQA
Linker sequence between decorin and heavy chain
(SEQ ID NO: 6):
SGGGGS
Decorin (SEQ ID NO: 7):
DEAAGIGPEVPDDRDFEPSLGPVCPFRCQCHLRVVQCSDLGLDKVPKDLPP
DTTLLDLQNNKITEIKDGDFKNLKNLHALILVNNKISKVSPGAFTPLVKLE
RLYLSKNQLKELPEKMPKTLQELRAHENEITKVRKVTFNGLNQMIVIELGT
NPLKSSGIENGAFQGMKKLSYIRIADTNITSIPQGLPPSLTELHLDGNKIS
RVDAASLKGLNNLAKLGLSFNSISAVDNGSLANTPHLRELHLDNNKLTRVP
GGLAEHKYIQVVYLHNNNISVVGSSDFCPPGHNTKKASYSGVSLFSNPVQY
WEIQPSTFRCVYVRSAIQLGNYK
Bevacizumab Heavy Chain Protein Sequence
(SEQ ID NO: 8):
EVQLVESGGGLVQPGGSLRLSCAASGYTFTNYGMNWVRQAPGKGLEWVGWI
NTYTGEPTYAADFKRRFTFSLDTSKSTAYLQMNSLRAEDTAVYYCAKYPHY
YGSSHWYFDVWGQGTLVTVSSASTKGPSVFPLAPSSKSTSGGTAALGCLVK
DYFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTL
PPSREEMTKNQVSLTCLVKGFYPSDIAVEWESNGQPENNYKTIPPVLDSDG
SFFLYSKLTVDKSRWQQGNVFSCSVMHEALHNHYTQKSLSLSPGK
Bevacizumab Light Chain Protein Sequence
(SEQ ID NO: 9):
DIQMTQSPSSLSASVGDRVTITCSASQDISNYLNWYQQKPGKAPKVLIYFT
SSLHSGVPSRFSGSGSGTDFTLTISSLQPEDFATYYCQQYSTVPWTFGQGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC

### Anti-CTLA4

Ipilimumab-Galacorin Fusion Heavy Chain Gene Sequence (SEQ ID NO: 10):
ATGATGTCCTTTGTCTCTCTGCTCCTGGTTGGCATCCTATTCCATGCCACCCAGGCCCAGGTGC
AGCTGGTGGAGTCCGGCGGCGGCGTCGTGCAGCCCGGCCGGTCCCTGCGGCTGTCCTGCGCCGC
CTCCGGCTTCACCTTCTCCTCCTACACCATGCACTGGGTGCGGCAGGCCCCCGGCAAGGGCCTG
GAGTGGGTGACTTTCATCTCCTACGACGGCAACAACAAGTACTACGCCGACTCCGTGAAGGGCC
GGTTCACCATCTCCCGCGACAACTCCAAGAACACCCTGTACCTGCAGATGAACTCCCTGCGGGC
CGAGGACACCGCCATCTACTACTGCGCCCGGACCGGCTGGCTGGGCCCCTTCGACTACTGGGGC
CAGGGCACCCTGGTGACCGTGTCCTCCGCCTCCACCAAGGGCCCATCGGTCTTCCCCCTGGCAC
CCTCTAGCAAGAGCACCTCTGGGGGCACAGCGGCCCTGGGCTGCCTGGTCAAGGACTACTTCCC
CGAACCGGTGACGGTGTCGTGGAACTCAGGCGCCCTGACCAGCGGCGTGCACACCTTCCCGGCT
GTCCTACAGTCCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTGGACAAGCGGGT
TGAGCCCAAATCTTGTGACAAAACTCACACATGCCCACCGTGCCCAGCACCTGAACTCCTGGGG
GGACCGTCAGTCTTCCTCTTCCCCCCAAAACCCAAGGACACCCTCATGATCTCCCGGACCCCTG
AGGTCACATGCGTGGTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGTACAACAGCACGTAC
CGTGTGGTCAGCGTCCTCACCGTCCTGCACCAGGACTGGCTGAATGGCAAGGAGTACAAGTGCA
AGGTCTCCAACAAAGCCCTCCCAGCCCCCATCGAGAAAACCATCTCCAAAGCCAAAGGGCAGCC
CCGAGAACCACAGGTGTACACCCTGCCTCCATCCCGCGATGAGCTGACCAAGAACCAGGTCAGC
CTGACCTGCCTGGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCTA
TAGCAAGCTCACCGTGGACAAGAGCAGGTGGCAGCAGGGGAACGTCTTCTCATGCTCCGTGATG

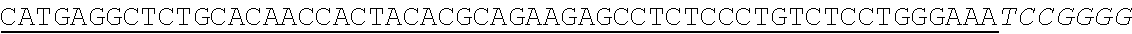

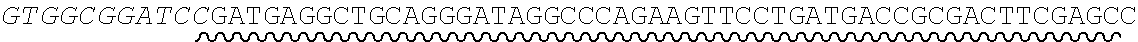

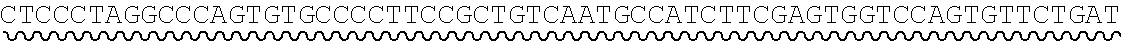

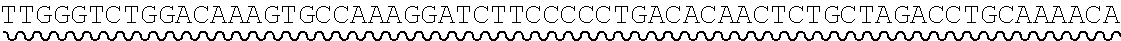

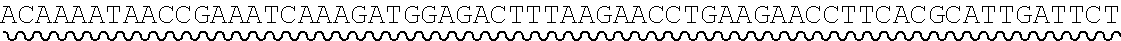

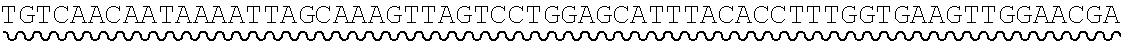

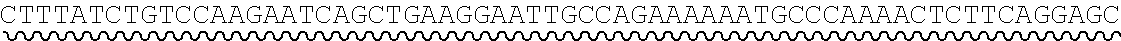

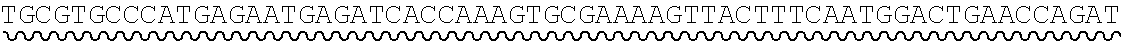

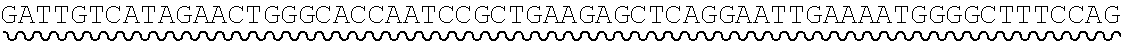

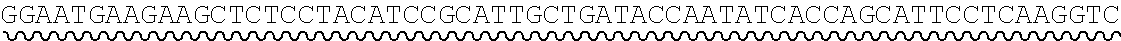

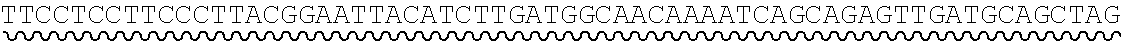

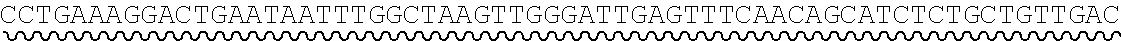

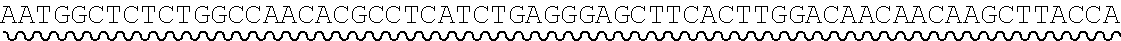

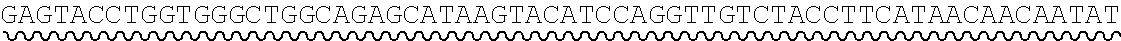

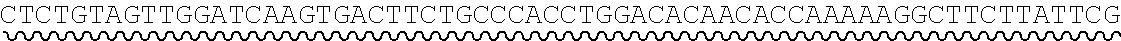

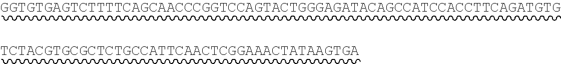

Signal peptide coding sequence is shown in boldface type. Heavy chain coding sequence is underlined with a straight line. Linker coding sequence is shown in italics. Galacorin coding sequence is underlined with a wavy line.

Ipilimumab-Galacorin Fusion Heavy Chain Protein Sequence (SEQ ID NO: 11):
MMSFVSLLLVGILFHATQAQVQLVESGGGVVQPGRSLRLSCAASGFTFSSYTMHWVRQAPGKGL
EWVTFISYDGNNKYYADSVKGRFTISRDNSKNTLYLQMNSLRAEDTAIYYCARTGWLGPFDYWG
QGTLVTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCPAPELLG
GPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSVM

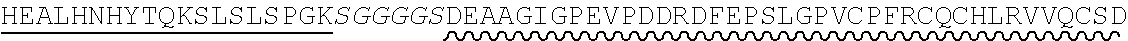

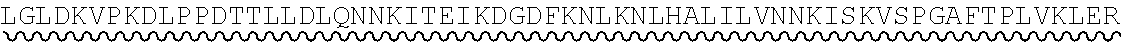

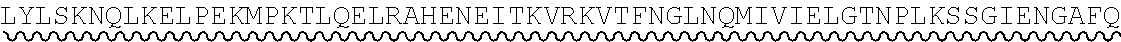

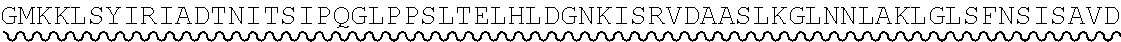

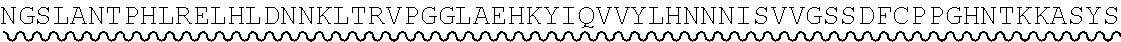

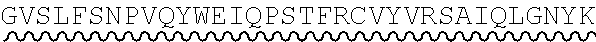

Signal peptide sequence is shown in boldface type. Heavy chain sequence is underlined with a straight line. Linker sequence is shown in italics. Galacorin sequence is underlined with a wavy line.

Ipilimumab Light Chain Gene Sequence
(SEQ ID NO: 12):
ATGATGTCCTTTGTCTCTCTGCTCCTGGTTGGCATCCTATTCCATGCCACC
CAGGCCGAGATCGTGCTGACCCAGTCCCCCGGCACCCTGTCCCTGTCCCCC
GGCGAGCGGGCCACCCTGTCCTGCCGGGCCTCCCAGTCCGTGGGCTCCTCC
TACCTGGCCTGGTACCAGCAGAAGCCCGGCCAGGCCCCCCGGCTGCTGATC
TACGGCGCCTTCTCCCGCGCCACCGGCATCCCCGACCGGTTCTCCGGCTCC
GGCTCCGGCACCGACTTCACCCTGACCATCTCCCGGCTGGAGCCCGAGGAC
TTCGCCGTGTACTACTGCCAGCAGTACGGCTCCTCCCCCTGGACCTTCGGC
CAGGGCACCAAGGTGGAGATCAAGCGAACTGTGGCTGCACCATCTGTCTTC
ATCTTCCCGCCATCTGATGAGCAGCTTAAGTCCGGAACTGCTAGCGTTGTG
TGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGAC
AGCAAGGACAGCACCTACAGCCTCAGCAGCACCCTGACGCTGAGCAAAGCA
GACTACGAGAAACACAAAGTCTACGCCTGCGAAGTCACCCATCAGGGCCTG
AGCTCGCCCGTCACAAAGAGCTTCAACAGGGGAGAGTGTTAG

Signal peptide coding sequence is shown in boldface type. Light chain coding sequence is underlined with a straight line.

Ipilimumab Light Chain Protein Sequence
(SEQ ID NO: 13):
MMSFVSLLLVGILFHATQAEIVLTQSPGTLSLSPGERATLSCRASQSVGSS
YLAWYQQKPGQAPRLLIYGAFSRATGIPDRFSGSGSGTDFTLTISRLEPED
FAVYYCQQYGSSPWTFGQGTKVEIKRTVAAPSVFIFPPSDEQLKSGTASVV
CLLNNFYPREAKVQWKVDNALQSGNSQESVTEQDSKDSTYSLSSTLTLSKA
DYEKHKVYACEVTHQGLSSPVTKSFNRGEC

Signal peptide sequence is shown in boldface type. Light chain sequence is underlined with a straight line.
Component amino acid sequences:
Signal sequence for heavy and light chains (SEQ ID NO:5)
Linker sequence between decorin and heavy chain (SEQ ID NO:6)
Decorin (SEQ ID NO:7)

Ipilimumab Heavy Chain Protein Sequence
(SEQ ID NO: 14):
QVQLVESGGGVVQPGRSLRLSCAASGFTFSSYTMHWVRQAPGKGLEWVTFI
SYDGNNKYYADSVKGRFTISRDNSKNTLYLQMNSLRAEDTAIYYCARTGWL
GPFDYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRD
ELTKNQVSLTCLVKGFYPSDIAVEWESNGQPENNYKTIPPVLDSDGSFFLY
SKLTVDKSRWQQGNVFSCSVMHEALHNHYTQKSLSLSPGK
Ipilimumab Light Chain Protein Sequence
(SEQ ID NO: 15):
EIVLTQSPGTLSLSPGERATLSCRASQSVGSSYLAWYQQKPGQAPRLLIYG
AFSRATGIPDRFSGSGSGTDFTLTISRLEPEDFAVYYCQQYGSSPWTFGQG
TKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSS
PVTKSFNRGEC

### Anti-PD-L1

Atezolizumab-Galacorin Fusion Heavy Chain Gene Sequence (SEQ ID NO: 16):
ATGATGTCCTTTGTCTCTCTGCTCCTGGTTGGCATCCTATTCCATGCCACCCAGGCCGAGGTAC
AACTCGTGGAATCCGGCGGGGGACTCGTCCAACCGGGTGGTAGTCTCAGGTTGAGCTGCGCTGC
GAGCGGTTTCACTTTCTCAGACTCATGGATTCATTGGGTGCGCCAAGCACCTGGAAAAGGGTTG
GAATGGGTTGCCTGGATCTCTCCTTACGGAGGTTCTACCTACTACGCTGATTCAGTAAAGGGGC
GGTTCACAATTTCAGCCGATACTTCCAAAAATACGGCTTACCTGCAAATGAACTCTTTGAGGGC
GGAGGATACGGCGGTCTACTACTGTGCACGCAGGCACTGGCCTGGAGGGTTCGATTATTGGGGT
CAAGGCACTTTGGTAACCGTATCCTCCGCTTCTACCAAAGGCCCATCAGTATTTCCTTTGGCTC
CCAGCTCTAAGTCCACTTCCGGTGGAACTGCCGCACTTGGATGTCTCGTCAAAGACTACTTTCC
TGAGCCGGTAACTGTGTCATGGAACTCCGGCGCCCTCACTAGCGGCGTCCATACATTTCCAGCG
GTTCTCCAGTCAAGTGGCCTCTACAGCCTGTCCAGTGTAGTTACTGTCCCGTCTTCTAGTCTGG
GAACGCAAACATATATTTGCAATGTGAATCATAAGCCTAGTAACACAAAAGTCGATAAAAAAGT
GGAGCCGAAAAGTTGTGACAAAACGCATACCTGTCCGCCTTGTCCGGCCCCCGAACTCTTGGGC
GGCCCATCAGTCTTTCTCTTCCCGCCCAAACCTAAGGACACGTTGATGATAAGTCGCACGCCCG
AGGTTACATGCGTCGTAGTCGATGTCAGCCACGAGGATCCGGAGGTAAAGTTTAACTGGTATGT
AGACGGAGTTGAAGTACACAACGCCAAAACTAAACCGAGAGAGGAGCAGTACGCATCAACCTAT
CGCGTAGTATCTGTATTGACGGTCCTTCACCAAGACTGGCTCAATGGGAAAGAATACAAGTGCA
AAGTTTCTAATAAAGCCCTCCCTGCACCAATCGAAAAGACTATTTCAAAAGCCAAAGGACAACC
AAGAGAACCACAAGTTTATACATTGCCACCTAGTCGCGAGGAGATGACTAAAAACCAAGTGTCC
CTTACTTGTCTCGTAAAGGGTTTCTATCCAAGCGACATAGCAGTTGAGTGGGAAAGTAATGGCC
AGCCGGAAAACAACTACAAGACGACCCCCCCGGTTCTCGACTCCGATGGATCATTCTTTTTGTA
TAGTAAACTCACAGTTGATAAGAGTCGATGGCAGCAGGGGAATGTTTTTTCTTGCTCTGTGATG
CACGAGGCGCTCCACAACCACTATACGCAAAAGTCCCTCAGCCTGAGCCCCGGGAAATCCGGGG

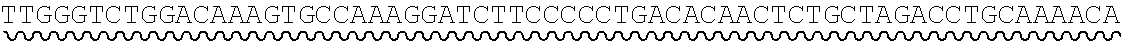

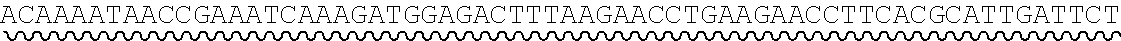

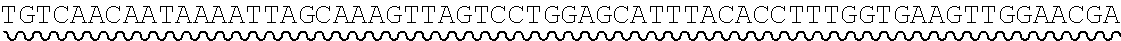

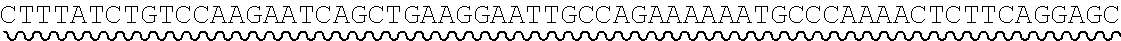

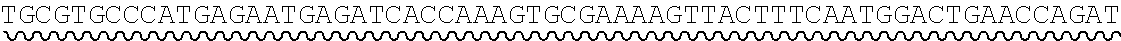

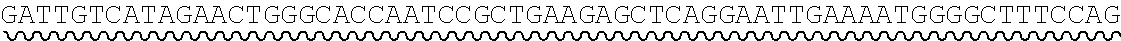

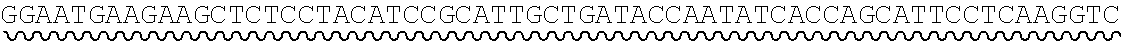

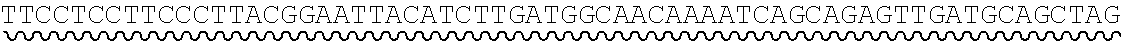

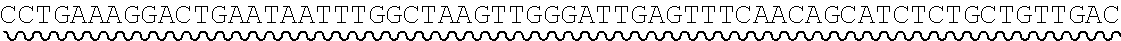

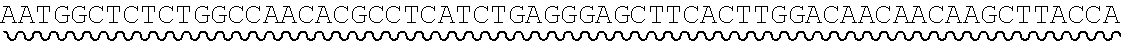

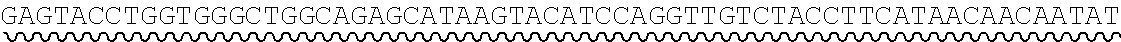

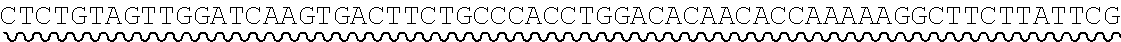

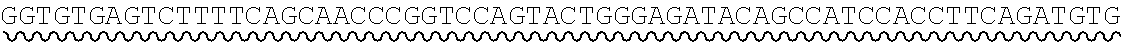

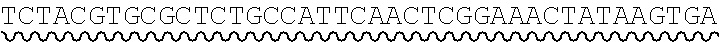

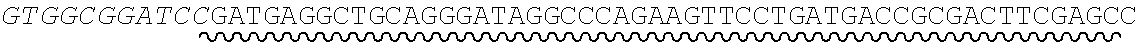

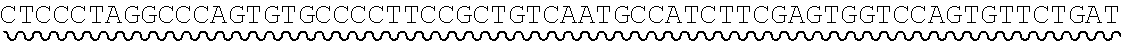

Signal peptide coding sequence is shown in boldface type. Heavy chain coding sequence is underlined with a straight line. Linker coding sequence is shown in italics. Galacorin coding sequence is underlined with a wavy line.

Atezolizumab-Galacorin Fusion Heavy Chain Protein Sequence (SEQ ID NO: 17):
MMSFVSLLLVGILFHATQAEVQLVESGGGLVQPGGSLRLSCAASGFTFSDSWIHWVRQAPGKGL
EWVAWISPYGGSTYYADSVKGRFTISADTSKNTAYLQMNSLRAEDTAVYYCARRHWPGGFDYWG
QGTLVTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVHNAKTKPREEQYASTY
RVVSVLTVLHQDWLNGKEYKCKVSNKALPAPIEKTISKAKGQPREPQVYTLPPSREEMTKNQVS
LTCLVKGFYPSDIAVEWESNGQPENNYKTIPPVLDSDGSFFLYSKLTVDKSRWQQGNVFSCSVM

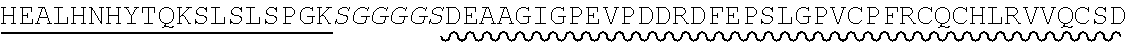

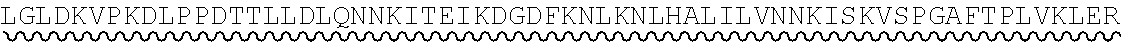

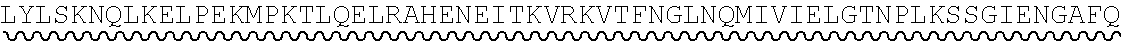

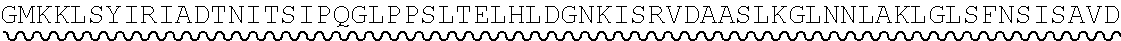

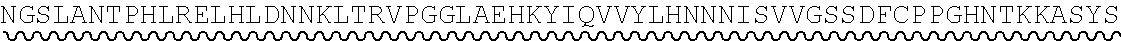

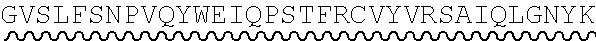

Signal peptide sequence is shown in boldface type. Heavy chain sequence is underlined with a straight line. Linker sequence is shown in italics. Galacorin sequence is underlined with a wavy line.

Atezolizumab Light Chain Gene Sequence
(SEQ ID NO: 18):
ATGATGTCCTTTGTCTCTCTGCTCCTGGTTGGCATCCTATTCCATGCCACC
CAGGCCGACATTCAGATGACACAATCACCTAGCAGTCTGAGTGCGAGCGTA
GGTGATCGCGTAACGATTACCTGCAGGGCCTCTCAAGACGTGTCAACGGCA
GTGGCGTGGTACCAGCAGAAGCCTGGTAAAGCTCCTAAGCTCCTCATCTAT
TCAGCTTCCTTCTTGTATAGTGGAGTACCGTCAAGATTTTCCGGAAGCGGA
TCAGGTACAGATTTTACTTTGACTATCAGTAGTTTGCAGCCAGAGGATTTC
GCTACATATTACTGTCAACAATATCTCTATCACCCTGCCACTTTTGGACAA
GGGACTAAAGTCGAAATAAAACGAACAGTGGCCGCACCAAGCGTTTTTATC
TTTCCCCCATCCGACGAGCAGTTGAAGAGCGGCACCGCGTCCGTGGTCTGC
CTGTTGAATAATTTCTATCCAAGGGAGGCAAAAGTGCAATGGAAAGTTGAT
AATGCGCTTCAATCCGGAAACTCACAAGAATCAGTAACAGAACAAGACTCT
AAAGACAGTACATATTCTCTTAGTAGCACACTCACTCTTTCAAAGGCTGAC
TATGAGAAACATAAAGTGTACGCTTGTGAAGTGACACATCAAGGTCTTAGC
TCCCCAGTAACTAAGAGCTTTAATAGGGGCGAGTGCTGA

Signal peptide coding sequence is shown in boldface type. Light chain coding sequence is underlined with a straight line.

Atezolizumab Light Chain Protein Sequence
(SEQ ID NO: 19):
MMSFVSLLLVGILFHATQADIQMTQSPSSLSASVGDRVTITCRASQDVSTA
VAWYQQKPGKAPKLLIYSASFLYSGVPSRFSGSGSGTDFTLTISSLQPEDF
ATYYCQQYLYHPATFGQGTKVEIKRTVAAPSVFIFPPSDEQLKSGTASVVC
LLNNFYPREAKVQWKVDNALQSGNSQESVTEQDSKDSTYSLSSTLTLSKAD
YEKHKVYACEVTHQGLSSPVTKSFNRGEC

Signal peptide sequence is shown in boldface type. Light chain sequence is underlined with a straight line.
Component amino acid sequences:
Signal sequence for heavy and light chains (SEQ ID NO:5)
Linker sequence between decorin and heavy chain (SEQ ID NO:6)
Decorin (SEQ ID NO:7)

Atezolizumab Heavy Chain Protein Sequence
(SEQ ID NO: 20):
EVQLVESGGGLVQPGGSLRLSCAASGFTFSDSWIHWVRQAPGKGLEW
VAWISPYGGSTYYADSVKGRFTISADTSKNTAYLQMNSLRAEDTAVY
YCARRHWPGGFDYWGQGTLVTVSSASTKGPSVFPLAPSSKSTSGGTA
ALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDG
VEVHNAKTKPREEQYASTYRVVSVLTVLHQDWLNGKEYKCKVSNKAL
PAPIEKTISKAKGQPREPQVYTLPPSREEMTKNQVSLTCLVKGFYPS
DIAVEWESNGQPENNYKTTPPVLDSDGSFFLYSKLTVDKSRWQQGNV
FSCSVMHEALHNHYTQKSLSLSPGK
Atezolizumab Light Chain Protein Sequence
(SEQ ID NO: 21):
DIQMTQSPSSLSASVGDRVTITCRASQDVSTAVAWYQQKPGKAPKLL
IYSASFLYSGVPSRFSGSGSGTDFTLTISSLQPEDFATYYCQQYLYH
PATFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC

### Anti-PD-L1

Avelumab-Galacorin Fusion Heavy Chain Gene Sequence (SEQ ID NO: 22):
ATGATGTCCTTTGTCTCTCTGCTCCTGGTTGGCATCCTATTCCATGCCACCCAGGCCGAGGTAC
AGCTTTTGGAGTCAGGCGGGGGGCTCGTCCAACCTGGGGGGTCACTCCGGTTGTCATGTGCTGC
CAGTGGCTTCACATTCTCATCTTACATTATGATGTGGGTTCGACAGGCCCCTGGGAAGGGGCTG
GAGTGGGTTTCCTCCATCTACCCCTCCGGGGGAATTACCTTCTATGCCGACACGGTAAAGGGTC
GCTTCACTATAAGTCGAGATAACAGCAAAAATACGCTGTATCTCCAGATGAACTCTCTCAGGGC
TGAGGACACAGCTGTATATTACTGCGCGCGGATTAAGTTGGGGACCGTCACAACAGTGGATTAC
TGGGGTCAAGGCACTCTGGTAACCGTATCCTCAGCATCCACCAAGGGCCCAAGTGTATTCCCGC
TGGCCCCTTCAAGTAAATCCACGTCTGGCGGCACAGCCGCTCTCGGTTGCCTGGTTAAGGACTA
CTTCCCAGAACCTGTCACTGTCAGTTGGAACTCAGGCGCATTGACATCTGGTGTCCATACATTC
CCCGCAGTCCTGCAAAGCTCTGGACTTTACAGTCTTAGTAGCGTAGTGACAGTCCCATCTTCAA
GTCTTGGGACCCAAACTTATATTTGCAACGTAAATCATAAACCCTCCAACACTAAAGTAGACAA
AAAAGTAGAGCCGAAATCTTGCGACAAAACGCATACATGCCCACCATGTCCCGCTCCGGAACTC
CTGGGCGGCCCGTCCGTTTTTCTCTTTCCCCCAAAGCCCAAGGATACGCTTATGATCAGCAGAA
CACCGGAAGTTACTTGTGTAGTCGTTGACGTGTCTCACGAAGATCCCGAAGTCAAATTTAATTG
GTATGTGGATGGCGTCGAAGTGCACAACGCAAAAACCAAACCCAGAGAGGAACAGTATAACAGC
ACGTATCGAGTGGTCTCCGTACTTACGGTCCTCCACCAGGACTGGTTGAATGGCAAGGAGTACA
AGTGCAAAGTGAGCAATAAAGCGTTGCCAGCCCCGATCGAAAAAACCATCAGCAAGGCCAAGGG
ACAGCCTAGAGAGCCGCAGGTTTACACCTTGCCGCCATCAAGGGATGAACTGACTAAAAACCAG
GTATCCCTGACCTGCCTGGTTAAGGGTTTTTACCCCAGTGATATAGCGGTTGAATGGGAGTCTA
ACGGGCAGCCAGAGAACAACTACAAAACGACACCTCCCGTTCTGGATTCCGATGGCAGCTTTTT
CTTGTATTCTAAACTCACCGTGGATAAATCCCGATGGCAGCAAGGCAACGTCTTCTCCTGCAGC
GTGATGCATGAAGCCTTGCACAACCACTATACCCAAAAGAGTCTCAGCCTGTCACCCGGGAAAT

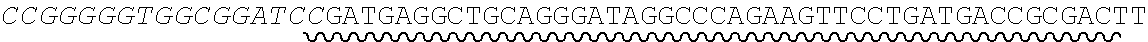

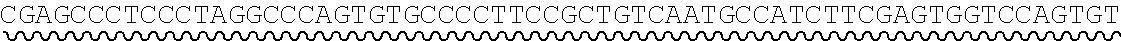

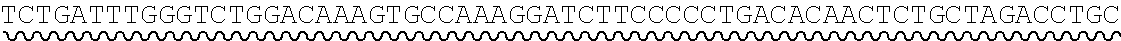

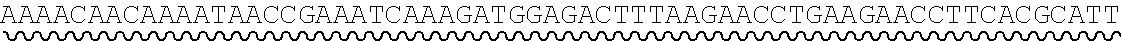

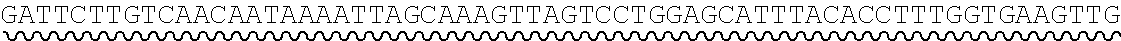

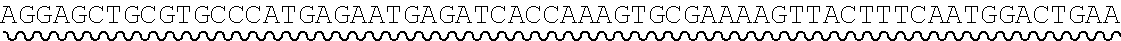

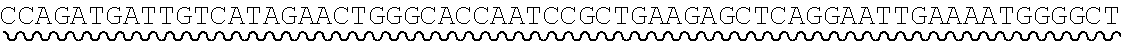

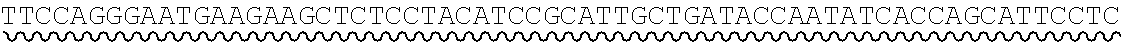

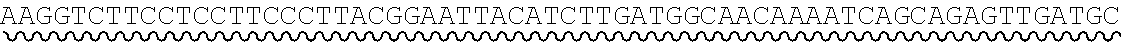

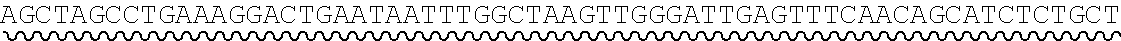

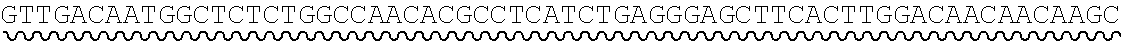

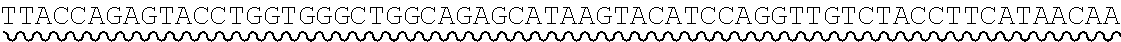

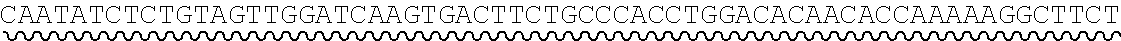

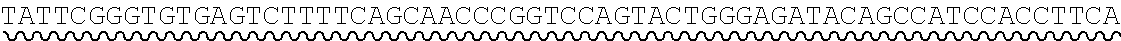

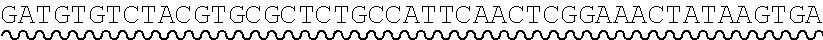

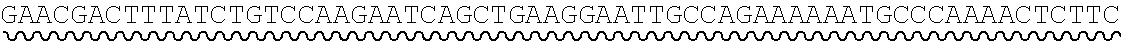

Signal peptide coding sequence is shown in boldface type. Heavy chain coding sequence is underlined with a straight line. Linker coding sequence is shown in italics. Galacorin coding sequence is underlined with a wavy line.

Avelumab-Galacorin Fusion Heavy Chain Protein Sequence (SEQ ID NO: 23):
MMSFVSLLLVGILFHATQAEVQLLESGGGLVQPGGSLRLSCAASGFTFSSYIMMWVRQAPGKGL
EWVSSIYPSGGITFYADTVKGRFTISRDNSKNTLYLQMNSLRAEDTAVYYCARIKLGTVTTVDY
WGQGTLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNVFSCS

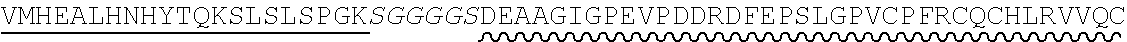

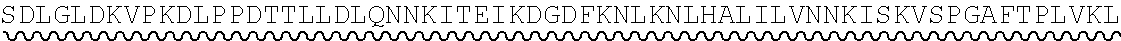

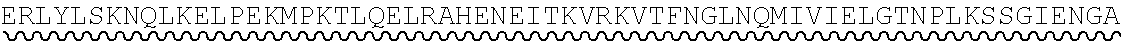

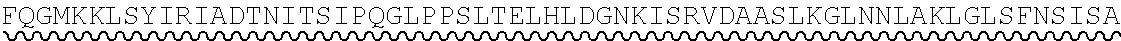

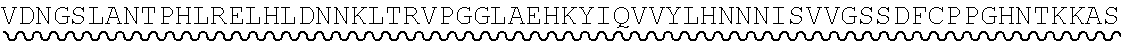

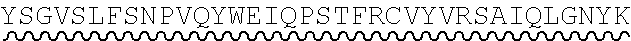

Signal peptide sequence is shown in boldface type. Heavy chain sequence is underlined with a straight line. Linker sequence is shown in italics. Galacorin sequence is underlined with a wavy line.

Avelumab Light Chain Gene Sequence
(SEQ ID NO: 24):
ATGATGTCCTTTGTCTCTCTGCTCCTGGTTGGCATCCTATTCCATGC
CACCCAGGCCCAGTCTGCACTTACACAACCGGCGTCCGTTTCCGGAT
CTCCAGGACAGAGCATTACTATCAGTTGCACGGGAACCTCCTCAGAC
GTAGGGGGGTATAATTATGTGTCTTGGTATCAACAGCATCCCGGGAA
AGCCCCCAAACTGATGATCTACGATGTCAGCAATAGACCAAGCGGTG
TGAGTAATCGATTTAGCGGGTCTAAATCTGGTAACACAGCATCCCTC
ACTATTAGTGGACTGCAAGCAGAAGATGAGGCAGACTATTATTGCAG
TAGCTATACGTCTAGTTCCACCCGCGTTTTTGGCACTGGGACGAAAG
TCACCGTTCTCGGACAACCAAAAGCAAACCCCACCGTGACTCTGTTT
CCGCCTAGCAGCGAAGAATTGCAGGCCAATAAGGCGACACTCGTATG
CCTTATCTCCGACTTCTACCCGGGCGCTGTGACAGTCGCGTGGAAAG
CCGACGGCAGCCCTGTTAAAGCTGGAGTCGAGACCACGAAGCCGTCC
AAGCAGAGTAACAATAAGTATGCTGCATCCAGTTATCTCTCTCTCAC
TCCGGAACAGTGGAAGTCCCATCGGTCCTATAGTTGCCAAGTGACCC
ATGAGGGTTCCACCGTAGAGAAAACGGTAGCACCTACCGAATGTAGT
TGA

Signal peptide coding sequence is shown in boldface type. Light chain coding sequence is underlined with a straight line.

Avelumab Light Chain Protein Sequence
(SEQ ID NO: 25):
MMSFVSLLLVGILFHATQAQSALTQPASVSGSPGQSITISCTGTSSD
VGGYNYVSWYQQHPGKAPKLMIYDVSNRPSGVSNRFSGSKSGNTASL
TISGLQAEDEADYYCSSYTSSSTRVFGTGTKVTVLGQPKANPTVTLF
PPSSEELQANKATLVCLISDFYPGAVTVAWKADGSPVKAGVETTKPS
KQSNNKYAASSYLSLTPEQWKSHRSYSCQVTHEGSTVEKTVAPTECS

Signal peptide sequence is shown in boldface type. Light chain sequence is underlined with a straight line.
Component amino acid sequences:
Signal sequence for heavy and light chains (SEQ ID NO:5)
Linker sequence between decorin and heavy chain (SEQ ID NO:6)
Decorin (SEQ ID NO:7)

Avelumab Heavy Chain Protein Sequence
(SEQ ID NO: 26):
EVQLLESGGGLVQPGGSLRLSCAASGFTFSSYIMMWVRQAPGKGLEW
VSSIYPSGGITFYADTVKGRFTISRDNSKNTLYLQMNSLRAEDTAVY
YCARIKLGTVTTVDYWGQGTLVTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNK
ALPAPIEKTISKAKGQPREPQVYTLPPSRDELTKNQVSLTCLVKGFY
PSDIAVEWESNGQPENNYKTTPPVLDSDGSFFLYSKLTVDKSRWQQG
NVFSCSVMHEALHNHYTQKSLSLSPGK
Avelumab Light Chain Protein Sequence
(SEQ ID NO: 27):
QSALTQPASVSGSPGQSITISCTGTSSDVGGYNYVSWYQQHPGKAPK
LMIYDVSNRPSGVSNRFSGSKSGNTASLTISGLQAEDEADYYCSSYT
SSSTRVFGTGTKVTVLGQPKANPTVTLFPPSSEELQANKATLVCLIS
DFYPGAVTVAWKADGSPVKAGVETTKPSKQSNNKYAASSYLSLTPEQ
WKSHRSYSCQVTHEGSTVEKTVAPTECS

### Anti-PD-L1

Durvalumab-Galacorin Fusion Heavy Chain Gene Sequence (SEQ ID NO: 28):
ATGATGTCCTTTGTCTCTCTGCTCCTGGTTGGCATCCTATTCCATGCCACCCAGGCCGAGGTCC
AGCTTGTTGAAAGCGGTGGTGGCCTCGTGCAGCCTGGTGGCAGTTTGCGGTTGTCTTGCGCAGC
TAGTGGTTTTACCTTCTCCAGATACTGGATGTCATGGGTCCGACAGGCCCCTGGCAAGGGGCTT
GAATGGGTTGCAAATATAAAGCAGGACGGTTCTGAAAAGTACTATGTAGACTCCGTCAAAGGAA
GATTTACTATTAGTCGAGACAACGCCAAGAATAGCCTCTACCTTCAGATGAATTCTTTGCGAGC
GGAGGACACAGCCGTATATTACTGCGCACGAGAAGGGGGGTGGTTCGGTGAACTGGCTTTTGAC
TACTGGGGGCAAGGTACGCTTGTCACGGTGAGCTCTGCCTCTACAAAGGGGCCGTCTGTGTTTC
CACTTGCTCCATCTAGTAAGTCAACTTCTGGAGGTACTGCGGCATTGGGATGCCTTGTTAAGGA
TTATTTTCCCGAACCTGTAACTGTGAGCTGGAATTCAGGTGCCCTCACCTCTGGTGTACATACC
TTTCCAGCAGTTTTGCAATCTTCCGGTTTGTACTCTCTTAGTTCAGTTGTAACTGTCCCCTCTT
CCTCTCTTGGTACCCAAACATACATTTGTAATGTCAATCACAAACCAAGCAATACCAAGGTAGA
CAAGCGGGTGGAACCCAAATCTTGTGACAAAACTCATACCTGCCCACCATGTCCCGCCCCGGAG
TTTGAAGGAGGTCCAAGTGTATTTCTTTTCCCGCCTAAGCCTAAGGATACCCTCATGATAAGTC
GGACACCAGAGGTGACGTGTGTTGTGGTAGACGTGAGTCACGAAGATCCCGAAGTTAAATTTAA
TTGGTATGTGGACGGGGTGGAAGTCCATAACGCGAAGACAAAGCCACGCGAAGAGCAGTACAAT
TCCACGTACAGGGTGGTTAGCGTGCTTACCGTCCTGCATCAAGATTGGCTGAACGGGAAAGAAT
ACAAATGCAAAGTATCCAACAAGGCGTTGCCTGCGAGTATCGAGAAAACGATTTCTAAAGCTAA
AGGACAACCCCGGGAACCCCAGGTCTATACACTGCCGCCCAGCAGAGAAGAGATGACGAAAAAT
CAAGTATCCCTTACGTGTCTCGTCAAAGGCTTCTACCCTTCCGATATTGCTGTAGAGTGGGAAT
CTAACGGGCAGCCGGAAAATAACTACAAGACTACTCCGCCAGTACTTGATTCAGACGGCTCCTT
CTTCCTTTATTCAAAACTCACCGTAGATAAAAGTAGGTGGCAACAAGGTAATGTTTTTAGCTGT
AGCGTAATGCACGAAGCGTTGCATAACCATTATACACAGAAATCACTCAGCCTGTCCCCCGGGA

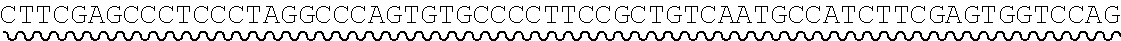

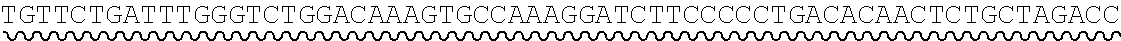

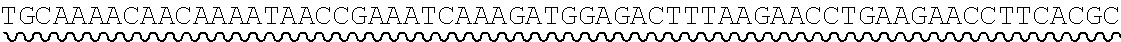

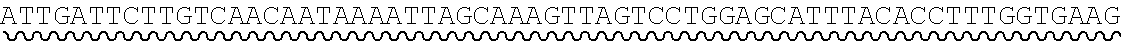

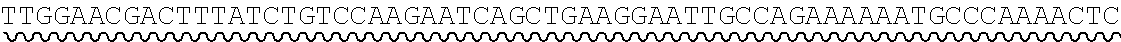

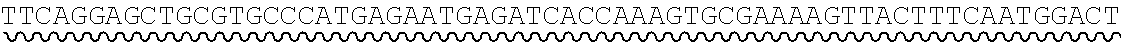

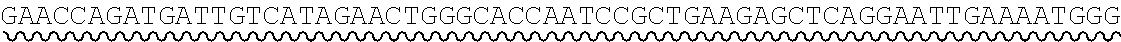

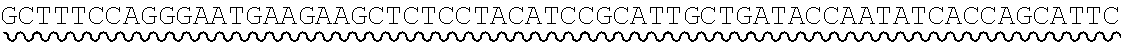

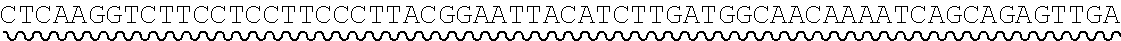

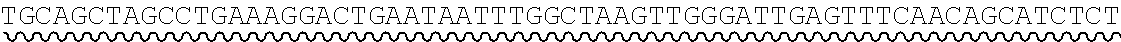

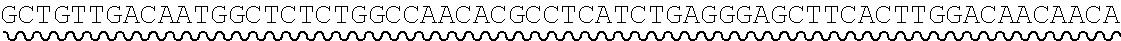

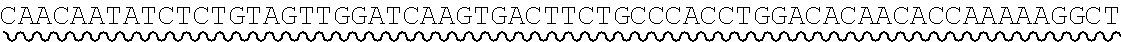

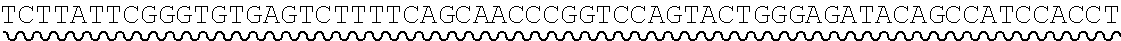

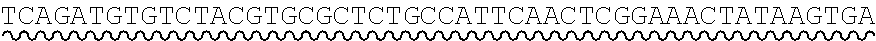

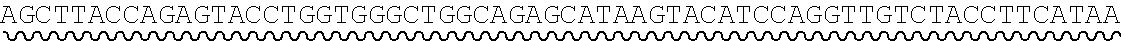

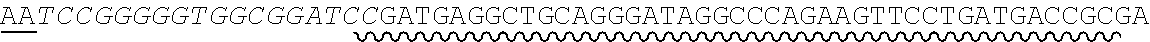

Signal peptide coding sequence is shown in boldface type. Heavy chain coding sequence is underlined with a straight line. Linker coding sequence is shown in italics. Galacorin coding sequence is underlined with a wavy line.

Durvalumab-Galacorin Fusion Heavy Chain Protein Sequence (SEQ ID NO: 29):
MMSFVSLLLVGILFHATQAEVQLVESGGGLVQPGGSLRLSCASGFTFSRYWMSWVRQAPGKGL
EWVANIKQDGSEKYYVDSVKGRFTISRDNAKNSLYLQMNSLRAEDTAVYYCAREGGWFGELAFD
YWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTHTCPPCPAPE
FEGGPSVFLFPPKPKDTLMISRTPEVTCVVVDVSHEDPEVKFNWYVDGVEVHNAKTKPREEQYN
STYRVVSVLTVLHQDWLNGKEYKCKVSNKALPASIEKTISKAKGQPREPQVYTLPPSREEMTKN
QVSLTCLVKGFYPSDIAVEWESNGQPENNYKTTPPVLDSDGSFFLYSKLTVDKSRWQQGNVFSC

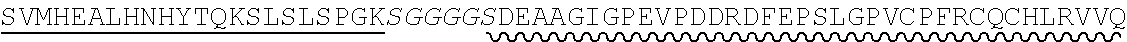

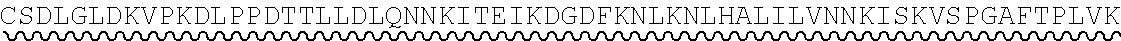

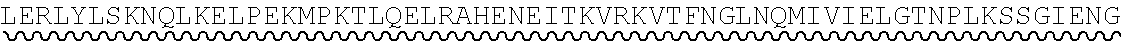

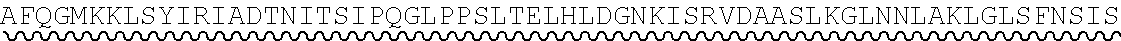

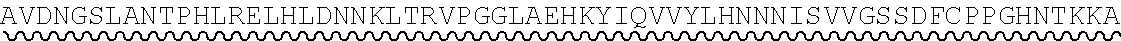

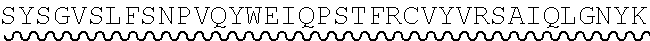

Signal peptide sequence is shown in boldface type. Heavy chain sequence is underlined with a straight line. Linker sequence is shown in italics. Galacorin sequence is underlined with a wavy line.

Durvalumab Light Chain Gene Sequence
(SEQ ID NO: 30):
ATGATGTCCTTTGTCTCTCTGCTCCTGGTTGGCATCCTATTCCATGC
CACCCAGGCCGAGATAGTTTTGACTCAAAGCCCTGGAACGCTCTCTT
TGTCTCCCGGCGAGCGGGCGACCCTTTCCTGTAGGGCTAGCCAGAGG
GTATCTTCCTCTTACCTGGCATGGTACCAGCAAAAGCCCGGACAAGC
CCCCCGACTTCTGATTTATGACGCCTCATCCCGGGCGACAGGCATCC
CTGACCGATTTTCAGGGAGTGGCTCTGGTACCGATTTTACGCTTACG
ATTTCCAGGCTGGAGCCCGAGGATTTCGCAGTGTATTACTGTCAACA
ATACGGCAGCTTGCCCTGGACCTTTGGACAAGGAACCAAGGTAGAGA
TCAAAAGGACCGTTGCCGCCCCGTCCGTGTTCATCTTCCCTCCGAGC
GATGAGCAACTTAAAAGTGGAACTGCAAGCGTTGTATGTCTTCTGAA
CAATTTCTATCCCCGAGAAGCCAAGGTACAGTGGAAAGTGGATAATG
CCCTCCAATCTGGCAATAGCCAAGAGTCTGTCACAGAGCAGGACAGC
AAGGACTCAACTTATTCACTTAGCTCCACCCTGACGCTGAGTAAAGC
AGACTACGAGAAGCATAAGGTGTATGCTTGTGAGGTTACACACCAAG
GCTTGTCTTCTCCTGTCACGAAGTCTTTCAATAGGGGCGAATGCTGA

Signal peptide coding sequence is shown in boldface type. Light chain coding sequence is underlined with a straight line.

Durvalumab Light Chain Protein Sequence
(SEQ ID NO: 31):
MMSFVSLLLVGILFHATQAEIVLTQSPGTLSLSPGERATLSCRASQR
VSSSYLAWYQQKPGQAPRLLIYDASSRATGIPDRFSGSGSGTDFTLT
ISRLEPEDFAVYYCQQYGSLPWTFGQGTKVEIKRTVAAPSVFIFPPS
DEQLKSGTASVVCLLNNFYPREAKVQWKVDNALQSGNSQESVTEQDS
KDSTYSLSSTLTLSKADYEKHKVYACEVTHQGLSSPVTKSFNRGEC

Signal peptide sequence is shown in boldface type. Light chain sequence is underlined with a straight line.
Component amino acid sequences:
Signal sequence for heavy and light chains (SEQ ID NO:5)
Linker sequence between decorin and heavy chain (SEQ ID NO:6)
Decorin (SEQ ID NO:7)

Durvalumab Heavy Chain Protein Sequence
(SEQ ID NO: 32):
EVQLVESGGGLVQPGGSLRLSCAASGFTFSRYWMSWVRQAPGKGLEW
VANIKQDGSEKYYVDSVKGRFTISRDNAKNSLYLQMNSLRAEDTAVY
YCAREGGWFGELAFDYWGQGTLVTVSSASTKGPSVFPLAPSSKSTSG
GTAALGCLVKDYFPEPVTVSWNSGALTSGVHTFPAVLQSSGLYSLSS
VVTVPSSSLGTQTYICNVNHKPSNTKVDKRVEPKSCDKTHTCPPCPA
PEFEGGPSVFLFPPKPKDTLMISRTPEVTCVVVDVSHEDPEVKFNWY
VDGVEVHNAKTKPREEQYNSTYRVVSVLTVLHQDWLNGKEYKCKVSN
KALPASIEKTISKAKGQPREPQVYTLPPSREEMTKNQVSLTCLVKGF
YPSDIAVEWESNGQPENNYKTTPPVLDSDGSFFLYSKLTVDKSRWQQ
GNVFSCSVMHEALHNHYTQKSLSLSPGK
Durvalumab Light Chain Protein Sequence
(SEQ ID NO: 33):
EIVLTQSPGTLSLSPGERATLSCRASQRVSSSYLAWYQQKPGQAPRL
LIYDASSRATGIPDRFSGSGSGTDFTLTISRLEPEDFAVYYCQQYGS
LPWTFGQGTKVEIKRTVAAPSVFIFPPSDEQLKSGTASVVCLLNNFY
PREAKVQWKVDNALQSGNSQESVTEQDSKDSTYSLSSTLTLSKADYE
KHKVYACEVTHQGLSSPVTKSFNRGEC

### Anti-PD-1

Nivolumab-Galacorin Fusion Heavy Chain Gene Sequence (SEQ ID NO: 34):
ATGATGTCCTTTGTCTCTCTGCTCCTGGTTGGCATCCTATTCCATGCCACCCAGGCCCAAGTCC
AGCTCGTGGAATCAGGAGGGGGTGTAGTCCAACCAGGGCGGAGCCTCCGACTTGATTGTAAAGC
ATCAGGAATTACATTTTCTAATAGCGGAATGCATTGGGTCCGACAAGCGCCAGGCAAGGGACTG
GAATGGGTCGCGGTGATATGGTATGATGGATCAAAACGCTATTATGCGGACTCTGTGAAGGGTC
GATTCACTATTAGCAGAGATAACAGCAAGAATACTCTCTTCCTTCAGATGAATTCACTTAGGGC
AGAGGACACAGCGGTGTACTATTGCGCGACGAACGACGATTATTGGGGCCAAGGGACATTGGTA
ACGGTGAGTTCTGCTAGTACTAAAGGGCCTTCCGTCTTCCCACTCGCCCCTTGTTCTAGAAGTA
CTAGTGAGTCAACAGCTGCTTTGGGTTGCTTGGTTAAAGACTACTTTCCTGAACCCGTGACTGT
GTCTTGGAATTCCGGTGCTCTTACTTCAGGTGTTCATACATTCCCAGCAGTATTGCAGAGCTCT
GGCTTGTATTCTCTCTCCTCAGTGGTGACAGTACCTTCCTCCTCTCTTGGCACAAAAACTTACA
CATGTAATGTAGACCATAAACCATCAAACACGAAAGTTGACAAGAGAGTAGAAAGCAAGTATGG
GCCTCCATGTCCCCCGTGCCCGGCGCCCGAGTTCCTGGGTGGTCCGTCAGTGTTCTTGTTCCCT
CCCAAGCCAAAAGATACATTGATGATAAGTCGGACGCCGGAGGTCACATGTGTAGTAGTTGATG
TCTCTCAGGAGGATCCTGAGGTGCAGTTTAACTGGTACGTCGATGGTGTTGAGGTACACAACGC
CAAAACTAAGCCGAGGGAAGAGCAGTTCAATTCAACATATCGGGTCGTGTCCGTATTGACAGTT
CTGCACCAAGATTGGTTGAACGGAAAAGAGTATAAGTGCAAAGTTAGCAATAAGGGACTTCCGT
CCTCAATTGAAAAAACCATTTCCAAAGCGAAAGGCCAACCTCGGGAACCTCAGGTATATACCTT
GCCACCCAGCCAAGAAGAAATGACTAAAAACCAGGTTAGTTTGACATGTTTGGTTAAAGGCTTT
TACCCGTCCGACATTGCCGTCGAGTGGGAAAGCAATGGGCAGCCTGAAAATAACTACAAGACAA
CCCCACCAGTATTGGATTCCGACGGTTCCTTCTTTCTTTACAGCCGCCTCACCGTCGATAAGAG
TCGGTGGCAAGAGGGGAATGTCTTTTCCTGTAGTGTCATGCACGAAGCACTTCACAACCATTAC

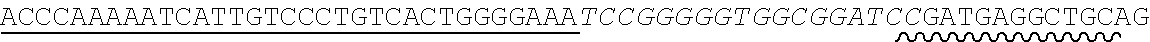

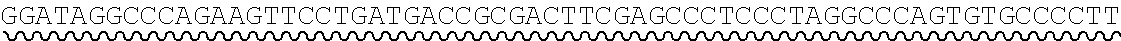

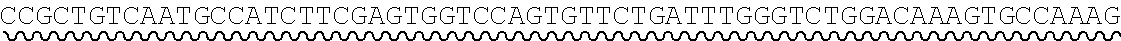

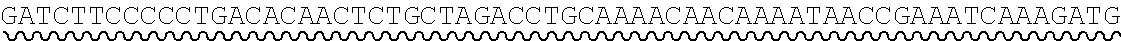

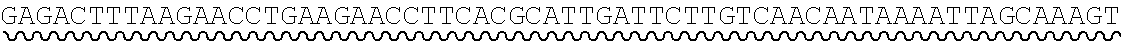

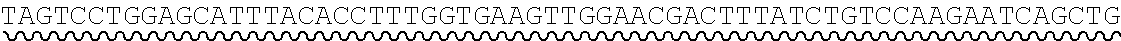

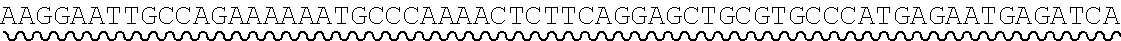

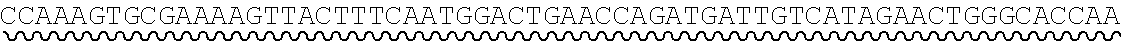

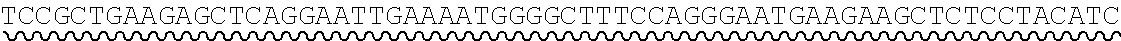

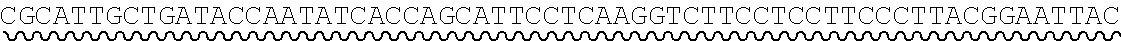

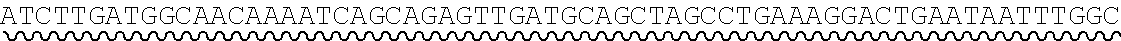

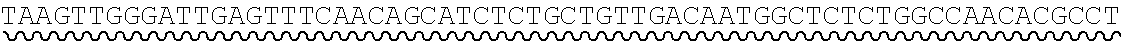

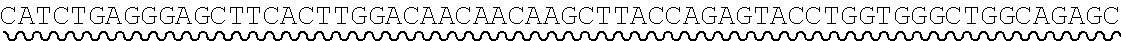

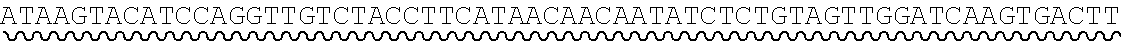

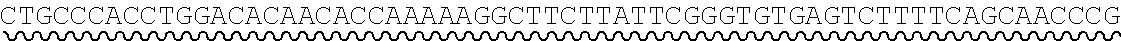

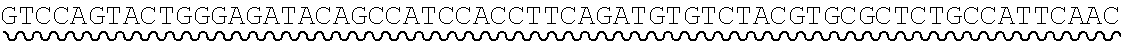

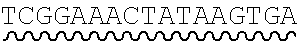

Signal peptide coding sequence is shown in boldface type. Heavy chain coding sequence is underlined with a straight line. Linker coding sequence is shown in italics. Galacorin coding sequence is underlined with a wavy line.

Nivolumab-Galacorin Fusion Heavy Chain Protein Sequence (SEQ ID NO: 35):
MMSFVSLLLVGILFHATQAQVQLVESGGGVVQPGRSLRLDCKASGITFSNSGMHWVRQAPGKGL
EWVAVIWYDGSKRYYADSVKGRFTISRDNSKNTLFLQMNSLRAEDTAVYYCATNDDYWGQGTLV
TVSSASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSVFLFP
PKPKDTLMISRTPEVTCVVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQEEMTKNQVSLTCLVKGF
YPSDIAVEWESNGQPENNYKTTPPVLDSDGSFFLYSRLTVDKSRWQEGNVFSCSVMHEALHNHY

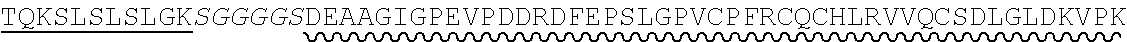

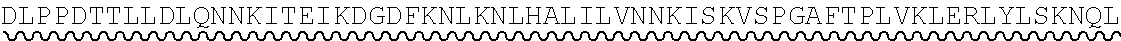

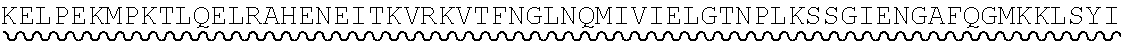

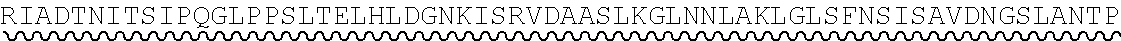

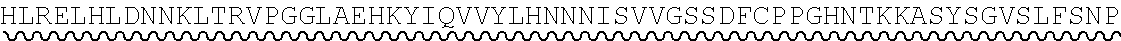

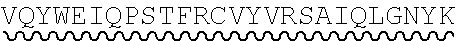

Signal peptide sequence is shown in boldface type. Heavy chain sequence is underlined with a straight line. Linker sequence is shown in italics. Galacorin sequence is underlined with a wavy line.

Nivolumab Light Chain Gene Sequence
(SEQ ID NO: 36):
ATGATGTCCTTTGTCTCTCTGCTCCTGGTTGGCATCCTATTCCATGC
CACCCAGGCCGAAATCGTATTGACTCAGTCCCCTGCTACACTTTCAC
TGAGTCCGGGTGAGCGGGCGACTTTGTCATGTCGGGCATCACAGAGT
GTAAGTAGTTATCTGGCCTGGTATCAACAGAAACCGGGACAGGCTCC
TCGCCTGCTGATTTATGACGCAAGCAATCGCGCGACCGGCATCCCGG
CGAGGTTCTCAGGGTCTGGATCAGGTACTGACTTTACCCTTACGATC
TCTTCTCTCGAACCTGAGGATTTCGCTGTCTATTACTGCCAGCAGTC
TTCTAACTGGCCGAGAACATTTGGTCAAGGGACAAAAGTCGAGATTA
AGCGAACTGTCGCAGCGCCATCTGTCTTTATCTTCCCTCCAAGCGAC
GAACAGCTTAAGAGTGGCACCGCCAGTGTTGTCTGCCTTCTGAATAA
CTTCTATCCAAGGGAAGCGAAAGTTCAGTGGAAGGTGGATAACGCTC
TGCAGTCTGGGAACTCTCAGGAAAGTGTAACAGAACAAGACTCCAAA
GACTCAACCTACTCTCTTAGTTCCACGTTGACCCTCTCCAAAGCGGA
CTATGAGAAGCATAAGGTCTACGCTTGCGAGGTAACACACCAGGGGC
TGAGTAGTCCGGTTACGAAGAGCTTCAACAGAGGGGAATGCTGA

Signal peptide coding sequence is shown in boldface type. Light chain coding sequence is underlined with a straight line.

Nivolumab Light Chain Protein Sequence
(SEQ ID NO: 37):
MMSFVSLLLVGILFHATQAEIVLTQSPATLSLSPGERATLSCRASQS
VSSYLAWYQQKPGQAPRLLIYDASNRATGIPARFSGSGSGTDFTLTI
SSLEPEDFAVYYCQQSSNWPRTFGQGTKVEIKRTVAAPSVFIFPPSD
EQLKSGTASVVCLLNNFYPREAKVQWKVDNALQSGNSQESVTEQDSK
DSTYSLSSTLTLSKADYEKHKVYACEVTHQGLSSPVTKSFNRGEC

Signal peptide sequence is shown in boldface type. Light chain sequence is underlined with a straight line.
Component amino acid sequences:
Signal sequence for heavy and light chains (SEQ ID NO:5)
Linker sequence between decorin and heavy chain (SEQ ID NO:6)
Decorin (SEQ ID NO:7)

Nivolumab Heavy Chain Protein Sequence
(SEQ ID NO: 38):
QVQLVESGGGVVQPGRSLRLDCKASGITFSNSGMHWVRQAPGKGLEW
VAVIWYDGSKRYYADSVKGRFTISRDNSKNTLFLQMNSLRAEDTAVY
YCATNDDYWGQGTLVTVSSASTKGPSVFPLAPCSRSTSESTAALGCL
VKDYFPEPVTVSWNSGALTSGVHTFPAVLQSSGLYSLSSVVTVPSSS
LGTKTYTCNVDHKPSNTKVDKRVESKYGPPCPPCPAPEFLGGPSVFL
FPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHE
ALHNHYTQKSLSLSLGK
Nivolumab Light Chain Protein Sequence
(SEQ ID NO: 39):
EIVLTQSPATLSLSPGERATLSCRASQSVSSYLAWYQQKPGQAPRLL
IYDASNRATGIPARFSGSGSGTDFTLTISSLEPEDFAVYYCQQSSNW
PRTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC

### Anti-PD-1

Pembrolizumab-Galacorin Fusion Heavy Chain Gene Sequence
(SEQ ID NO: 40):
ATGATGTCCTTTGTCTCTCTGCTCCTGGTTGGCATCCTATTCCATGCCACCCAGGCCCAGGTGC
AATTGGTCCAAAGTGGGGTCGAGGTCAAGAAGCCAGGAGCTTCTGTAAAAGTTTCATGTAAGGC
ATCTGGGTATACCTTCACGAACTACTATATGTATTGGGTGCGCCAAGCGCCAGGGCAGGGCCTC
GAATGGATGGGTGGCATCAATCCGAGCAACGGGGGCACCAACTTTAACGAAAAGTTTAAAAACC
GGGTCACCTTGACAACGGACAGTAGCACGACTACCGCTTATATGGAGCTGAAGAGTTTGCAGTT
TGATGATACTGCGGTTTATTATTGTGCACGCAGAGATTATAGGTTCGACATGGGCTTCGACTAC
TGGGGTCAAGGTACTACGGTAACTGTATCATCTGCTAGTACAAAGGGCCCTTCCGTTTTCCCCC
TCGCCCCGTGCAGCCGCTCAACGTCCGAAAGCACCGCTGCACTTGGGTGCCTTGTAAAAGACTA
TTTTCCAGAGCCAGTTACCGTGTCTTGGAATAGTGGCGCACTTACGTCCGGGGTGCACACTTTT
CCGGCTGTCTTGCAATCCTCTGGACTCTATTCCTTGAGTAGCGTCGTAACAGTACCAAGTAGTA
GTCTCGGCACCAAAACGTATACGTGCAATGTTGATCATAAGCCTAGCAACACGAAAGTTGACAA
AAGAGTTGAGAGTAAATATGGACCCCCCTGTCCGCCATGCCCGGCCCCTGAATTCCTTGGGGGC
CCGTCTGTATTTCTTTTCCCGCCCAAGCCGAAGGATACACTGATGATAAGCAGAACGCCTGAGG
TTACCTGCGTCGTGGTCGACGTAAGCCAGGAAGATCCTGAGGTGCAATTTAATTGGTATGTGGA
CGGGGTCGAGGTTCATAATGCAAAAACAAAACCCCGAGAGGAGCAATTTAATTCAACGTACAGA
GTCGTTAGCGTACTTACAGTGCTGCACCAGGATTGGCTCAACGGGAAGGAGTATAAGTGCAAGG
TGTCTAATAAAGGTTTGCCCTCCAGTATAGAAAAAACCATCTCAAAGGCGAAAGGACAGCCTAG
AGAACCTCAAGTATATACCCTCCCACCCTCCCAAGAAGAGATGACAAAGAACCAAGTGAGTCTC
ACATGCCTCGTCAAGGGTTTCTACCCAAGCGATATAGCCGTAGAGTGGGAATCAAATGGTCAGC
CGGAGAATAACTACAAAACTACTCCGCCAGTCTTGGATAGCGACGGGTCTTTTTTCCTGTACTC
TAGGCTGACGGTGGACAAGTCAAGATGGCAGGAAGGAAATGTTTTTAGCTGCTCCGTTATGCAT
GAGGCTCTCCACAATCATTATACACAAAAAAGTTTGTCTCTGTCATTGGGGAAATCCGGGGGTG

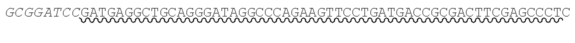

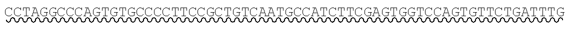

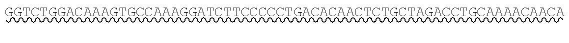

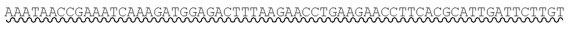

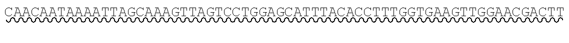

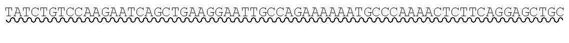

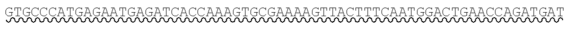

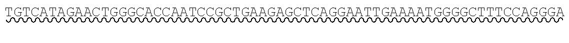

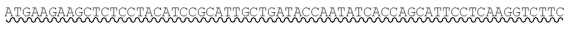

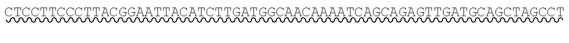

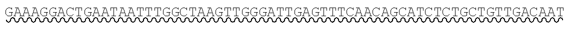

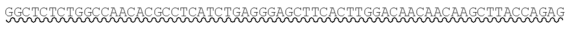

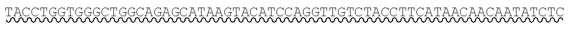

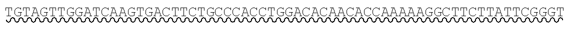

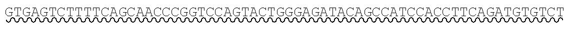

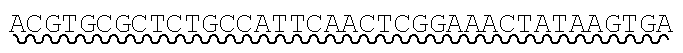

Signal peptide coding sequence is shown in boldface type. Heavy chain coding sequence is underlined with a straight line. Linker coding sequence is shown in italics. Galacorin coding sequence is underlined with a wavy line.

Pembrolizumab-Galacorin Fusion Heavy Chain Protein Sequence
(SEQ ID NO: 41):
MMSFVSLLLVGILFHATQAQVQLVQSGVEVKKPGASVKVSCKASGYTFTNYYMYWVRQAPGQGL
EWMGGINPSNGGTNFNEKFKNRVTLTTDSSTTTAYMELKSLQFDDTAVYYCARRDYRFDMGFDY
WGQGTTVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPEFLGG
PSVFLFPPKPKDTLMISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTLPPSQEEMTKNQVSL
TCLVKGFYPSDIAVEWESNGQPENNYKTIPPVLDSDGSFFLYSRLTVDKSRWQEGNVFSCSVMH

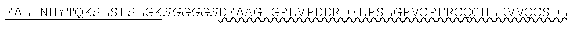

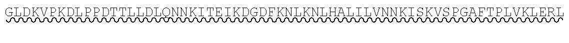

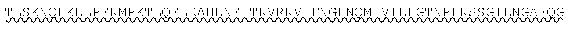

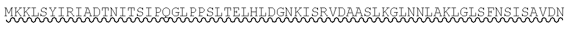

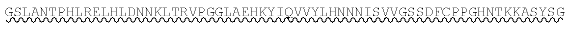

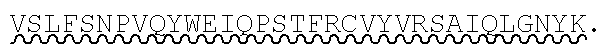

Signal peptide sequence is shown in boldface type. Heavy chain sequence is underlined with a straight line. Linker sequence is shown in italics. Galacorin sequence is underlined with a wavy line.

Pembrolizumab Light Chain Gene Sequence
(SEQ ID NO: 42):
ATGATGTCCTTTGTCTCTCTGCTCCTGGTTGGCATCCTATTCCATGC
CACCCAGGCCGAAATTGTTCTTACGCAGAGTCCAGCAACTTTGTCAC
TCTCTCCCGGCGAACGAGCGACATTGTCCTGTCGCGCGAGTAAGGGT
GTCTCAACATCTGGATACTCATATCTGCATTGGTACCAGCAAAAACC
GGGACAAGCGCCGCGATTGCTGATTTATCTCGCCTCCTACCTTGAAA
GTGGTGTGCCTGCGAGGTTCTCCGGTAGTGGATCAGGCACCGATTTC
ACTTTGACCATCAGCAGCCTCGAACCAGAAGATTTTGCCGTCTACTA
CTGCCAACATAGCAGGGATTTGCCACTGACATTCGGCGGGGGTACGA
AAGTTGAGATTAAACGGACTGTAGCGGCACCTTCTGTCTTCATCTTC
CCACCAAGCGATGAGCAGCTTAAAAGCGGTACAGCTTCAGTGGTGTG
CCTTTTGAACAACTTTTATCCGCGAGAAGCCAAGGTCCAATGGAAGG
TGGATAACGCTTTGCAATCCGGTAACTCACAGGAGTCAGTAACAGAG
CAAGATAGTAAAGATAGCACGTATTCACTTAGCAGTACCCTTACTCT
TAGCAAGGCTGATTATGAAAAACATAAGGTATATGCGTGCGAGGTAA
CGCACCAAGGACTTAGCTCCCCAGTGACGAAGTCATTTAACCGGGGG
GAGTGCTGA

Signal peptide coding sequence is shown in boldface type. Light chain coding sequence is underlined with a straight line.

Pembrolizumab Light Chain Protein Sequence
(SEQ ID NO: 43):
MMSFVSLLLVGILFHATQAEIVLTQSPATLSLSPGERATLSCRASKG
VSTSGYSYLHWYQQKPGQAPRLLIYLASYLESGVPARFSGSGSGTDF
TLTISSLEPEDFAVYYCQHSRDLPLTFGGGTKVEIKRTVAAPSVFIF
PPSDEQLKSGTASVVCLLNNFYPREAKVQWKVDNALQSGNSQESVTE
QDSKDSTYSLSSTLTLSKADYEKHKVYACEVTHQGLSSPVTKSFNRG
EC.

Signal peptide sequence is shown in boldface type. Light chain sequence is underlined with a straight line.
Component amino acid sequences:
Signal sequence for heavy and light chains (SEQ ID NO:5)
Linker sequence between decorin and heavy chain (SEQ ID NO:6)

Pembrolizumab Heavy Chain Protein Sequence
(SEQ ID NO: 44):
QVQLVQSGVEVKKPGASVKVSCKASGYTFTNYYMYWVRQAPGQGLEW
MGGINPSNGGTNFNEKFKNRVTLTTDSSTTTAYMELKSLQFDDTAVY
YCARRDYRFDMGFDYWGQGTTVTVSSASTKGPSVFPLAPCSRSTSES
TAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFL
GGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLP
SSIEKTISKAKGQPREPQVYTLPPSQEEMTKNQVSLTCLVKGFYPSD
IAVEWESNGQPENNYKTTPPVLDSDGSFFLYSRLTVDKSRWQEGNVF
SCSVMHEALHNHYTQKSLSLSLGK
Pembrolizumab Light Chain Protein Sequence
(SEQ ID NO: 45):
EIVLTQSPATLSLSPGERATLSCRASKGVSTSGYSYLHWYQQKPGQA
PRLLIYLASYLESGVPARFSGSGSGTDFTLTISSLEPEDFAVYYCQH
SRDLPLTFGGGTKVEIKRTVAAPSVFIFPPSDEQLKSGTASVVCLLN
NFYPREAKVQWKVDNALQSGNSQESVTEQDSKDSTYSLSSTLTLSKA
DYEKHKVYACEVTHQGLSSPVTKSFNRGEC

### Anti-PD-L1

Avelumab-Galacorin Fusion Heavy Chain Gene Sequence
(SEQ ID NO: 50):
ATGATGTCCTTTGTCTCTCTGCTCCTGGTTGGCATCCTATTCCATGCCACCCAGGCCGAGGTAC
AGCTTTTGGAGTCAGGCGGGGGGCTCGTCCAACCTGGGGGGTCACTCCGGTTGTCATGTGCTGC
CAGTGGCTTCACATTCTCATCTTACATTATGATGTGGGTTCGACAGGCCCCTGGGAAGGGGCTG
GAGTGGGTTTCCTCCATCTACCCCTCCGGGGGAATTACCTTCTATGCCGACACGGTAAAGGGTC
GCTTCACTATAAGTCGAGATAACAGCAAAAATACGCTGTATCTCCAGATGAACTCTCTCAGGGC
TGAGGACACAGCTGTATATTACTGCGCGCGGATTAAGTTGGGGACCGTCACAACAGTGGATTAC
TGGGGTCAAGGCACTCTGGTAACCGTATCCTCAGCATCCACCAAGGGCCCAAGTGTATTCCCGC
TGGCCCCTTCAAGTAAATCCACGTCTGGCGGCACAGCCGCTCTCGGTTGCCTGGTTAAGGACTA
CTTCCCAGAACCTGTCACTGTCAGTTGGAACTCAGGCGCATTGACATCTGGTGTCCATACATTC
CCCGCAGTCCTGCAAAGCTCTGGACTTTACAGTCTTAGTAGCGTAGTGACAGTCCCATCTTCAA
GTCTTGGGACCCAAACTTATATTTGCAACGTAAATCATAAACCCTCCAACACTAAAGTAGACAA
AAAAGTAGAGCCGAAATCTIGCGACAAAACGCATACATGCCCACCATGTCCCGCTCCGGAACTC
CTGGGCGGCCCGTCCGTTTTTCTCTTTCCCCCAAAGCCCAAGGATACGCTTATGATCAGCAGAA
CACCGGAAGTTACTTGTGTAGTCGTTGACGTGTCTCACGAAGATCCCGAAGTCAAATTTAATTG
GTATGTGGATGGCGTCGAAGTGCACAACGCAAAAACCAAACCCAGAGAGGAACAGTATAACAGC
ACGTATCGAGTGGTCTCCGTACTTACGGTCCTCCACCAGGACTGGTTGAATGGCAAGGAGTACA
AGTGCAAAGTGAGCAATAAAGCGTTGCCAGCCCCGATCGAAAAAACCATCAGCAAGGCCAAGGG
ACAGCCTAGAGAGCCGCAGGTTTACACCTTGCCGCCATCAAGGGATGAACTGACTAAAAACCAG
GTATCCCTGACCTGCCTGGTTAAGGGTTTTTACCCCAGTGATATAGCGGTTGAATGGGAGTCTA
ACGGGCAGCCAGAGAACAACTACAAAACGACACCTCCCGTTCTGGATTCCGATGGCAGCTTTTT
CTTGTATTCTAAACTCACCGTGGATAAATCCCGATGGCAGCAAGGCAACGTCTTCTCCTGCAGC
GTGATGCATGAAGCCTTGCACAACCACTATACCCAAAAGAGTCTCAGCCTGTCACCCGGGAAAT

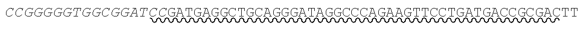

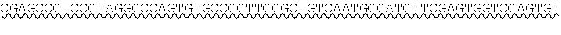

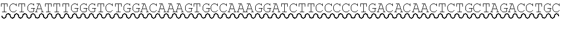

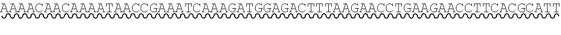

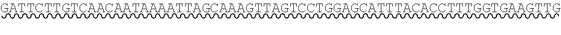

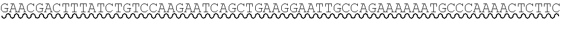

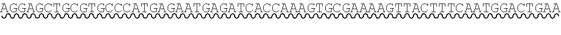

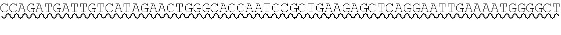

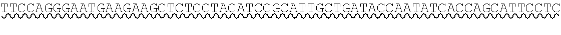

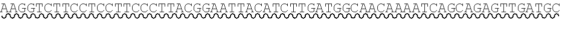

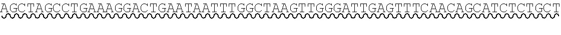

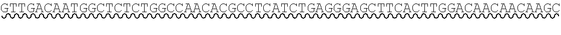

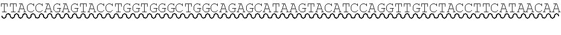

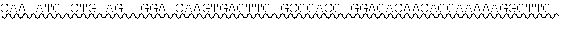

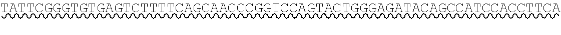

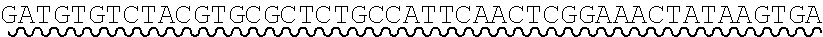

Signal peptide coding sequence is shown in boldface type. Heavy chain coding sequence is underlined with a straight line. Linker coding sequence is shown in italics. Galacorin coding sequence is underlined with a wavy line.

Avelumab-Galacorin Fusion Heavy Chain Protein Sequence
(SEQ ID NO: 51):
MMSFVSLLLVGILFHATQAEVQLLESGGGLVQPGGSLRLSCAASGFTFSSYIMMWVRQAPGKGL
EWVSSIYPSGGITFYADTVKGRFTISRDNSKNTLYLQMNSLRAEDTAVYYCARIKLGTVTTVDY
WGQGTLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNVFSCS

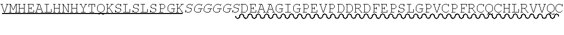

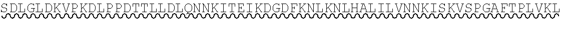

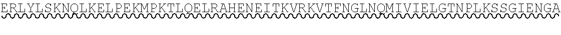

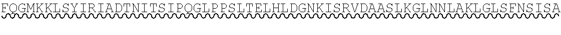

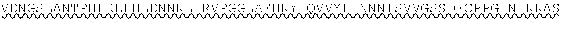

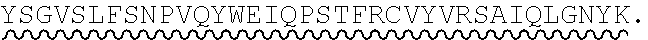

Signal peptide sequence is shown in boldface type. Heavy chain sequence is underlined with a straight line. Linker sequence is shown in italics. Galacorin sequence is underlined with a wavy line.

Avelumab Light Chain Gene Sequence
(SEQ ID NO: 52):
ATGATGTCCTTTGTCTCTCTGCTCCTGGTTGGCATCCTATTCCATGC
CACCCAGGCCCAGTCTGCACTTACACAACCGGCGTCCGTTTCCGGAT
CTCCAGGACAGAGCATTACTATCAGTTGCACGGGAACCTCCTCAGAC
GTAGGGGGGTATAATTATGTGTCTTGGTATCAACAGCATCCCGGGAA
AGCCCCCAAACTGATGATCTACGATGTCAGCAATAGACCAAGCGGTG
TGAGTAATCGATTTAGCGGGTCTAAATCTGGTAACACAGCATCCCTC
ACTATTAGTGGACTGCAAGCAGAAGATGAGGCAGACTATTATTGCAG
TAGCTATACGTCTAGTTCCACCCGCGTTTTTGGCACTGGGACGAAAG
TCACCGTTCTCGGACAACCAAAAGCAAACCCCACCGTGACTCTGTTT
CCGCCTAGCAGCGAAGAATTGCAGGCCAATAAGGCGACACTCGTATG
CCTTATCTCCGACTTCTACCCGGGCGCTGTGACAGTCGCGTGGAAAG
CCGACGGCAGCCCTGTTAAAGCTGGAGTCGAGACCACGAAGCCGTCC
AAGCAGAGTAACAATAAGTATGCTGCATCCAGTTATCTCTCTCTCAC
TCCGGAACAGTGGAAGTCCCATCGGTCCTATAGTTGCCAAGTGACCC
ATGAGGGTTCCACCGTAGAGAAAACGGTAGCACCTACCGAATGTAGT
TGA

Signal peptide coding sequence is shown in boldface type. Light chain coding sequence is underlined with a straight line.

Avelumab Light Chain Protein Sequence
(SEQ ID NO: 53):
MMSFVSLLLVGILFHATQAQSALTQPASVSGSPGQSITISCTGTSSD
VGGYNYVSWYQQHPGKAPKLMIYDVSNRPSGVSNRFSGSKSGNTASL
TISGLQAEDEADYYCSSYTSSSTRVFGTGTKVTVLGQPKANPTVTLF
PPSSEELQANKATLVCLISDFYPGAVTVAWKADGSPVKAGVETTKPS
KQSNNKYAASSYLSLTPEQWKSHRSYSCQVTHEGSTVEKTVAPTECS.

Signal peptide sequence is shown in boldface type. Light chain sequence is underlined with a straight line.

### Anti-PD-L1

In some embodiments, constructs comprise two or more copies of galacorin or other decorin molecule attached to each C heavy chain gene or a portion of the galacorin molecule (e.g., TGF-Beta binding moiety) attached to the heavy chain gene as a single entity or as two or more copies. There are multiple TGF-Beta binding domains in the Galacorin/Decorin molecule. These domains are configured in any suitable configuration. In some embodiments, each of the above-described options is attached to a bi-or multi specific antibody directed at 2 or more targets. Exemplary sequences are shown below.

Avelumab-Galacorin2x Fusion Heavy Chain Gene Sequence
(SEQ ID NO: 54):
ATGATGTCCTTTGTCTCTCTGCTCCTGGTTGGCATCCTATTCCATGCCACCCAGGCCGAGGTAC
AGCTTTTGGAGTCAGGCGGGGGGCTCGTCCAACCTGGGGGGTCACTCCGGTTGTCATGTGCTGC
CAGTGGCTTCACATTCTCATCTTACATTATGATGTGGGTTCGACAGGCCCCTGGGAAGGGGCTG
GAGTGGGTTTCCTCCATCTACCCCTCCGGGGGAATTACCTTCTATGCCGACACGGTAAAGGGTC
GCTTCACTATAAGTCGAGATAACAGCAAAAATACGCTGTATCTCCAGATGAACTCTCTCAGGGC
TGAGGACACAGCTGTATATTACTGCGCGCGGATTAAGTTGGGGACCGTCACAACAGTGGATTAC
TGGGGTCAAGGCACTCTGGTAACCGTATCCTCAGCATCCACCAAGGGCCCAAGTGTATTCCCGC
TGGCCCCTTCAAGTAAATCCACGTCTGGCGGCACAGCCGCTCTCGGTTGCCTGGTTAAGGACTA
CTTCCCAGAACCTGTCACTGTCAGTTGGAACTCAGGCGCATTGACATCTGGTGTCCATACATTC
CCCGCAGTCCTGCAAAGCTCTGGACTTTACAGTCTTAGTAGCGTAGTGACAGTCCCATCTTCAA
GTCTTGGGACCCAAACTTATATTTGCAACGTAAATCATAAACCCTCCAACACTAAAGTAGACAA
AAAAGTAGAGCCGAAATCTIGCGACAAAACGCATACATGCCCACCATGTCCCGCTCCGGAACTC
CTGGGCGGCCCGTCCGTTTTTCTCTTTCCCCCAAAGCCCAAGGATACGCTTATGATCAGCAGAA
CACCGGAAGTTACTTGTGTAGTCGTTGACGTGTCTCACGAAGATCCCGAAGTCAAATTTAATTG
GTATGTGGATGGCGTCGAAGTGCACAACGCAAAAACCAAACCCAGAGAGGAACAGTATAACAGC
ACGTATCGAGTGGTCTCCGTACTTACGGTCCTCCACCAGGACTGGTTGAATGGCAAGGAGTACA
AGTGCAAAGTGAGCAATAAAGCGTTGCCAGCCCCGATCGAAAAAACCATCAGCAAGGCCAAGGG
ACAGCCTAGAGAGCCGCAGGTTTACACCTTGCCGCCATCAAGGGATGAACTGACTAAAAACCAG
GTATCCCTGACCTGCCTGGTTAAGGGTTTTTACCCCAGTGATATAGCGGTTGAATGGGAGTCTA
ACGGGCAGCCAGAGAACAACTACAAAACGACACCTCCCGTTCTGGATTCCGATGGCAGCTTTTT
CTTGTATTCTAAACTCACCGTGGATAAATCCCGATGGCAGCAAGGCAACGTCTTCTCCTGCAGC
GTGATGCATGAAGCCTTGCACAACCACTATACCCAAAAGAGTCTCAGCCTGTCACCCGGGAAAT

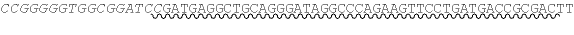

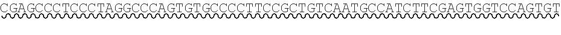

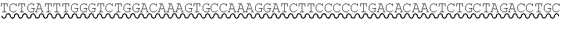

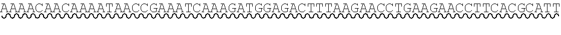

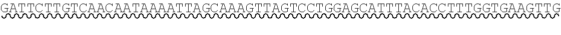

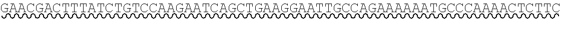

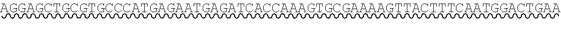

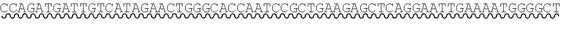

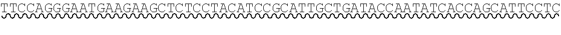

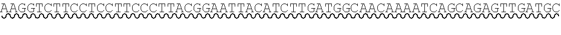

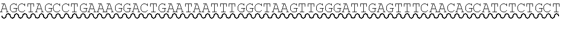

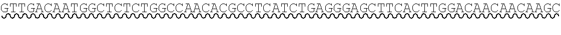

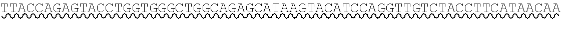

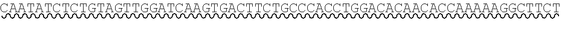

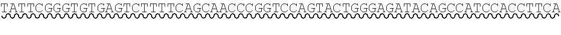

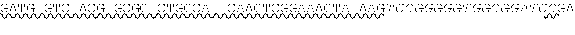

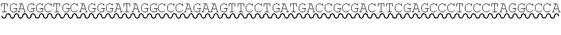

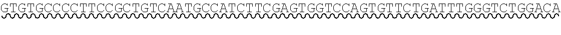

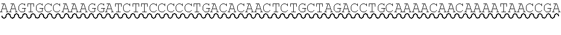

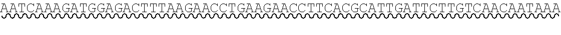

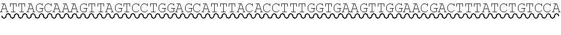

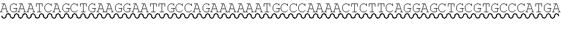

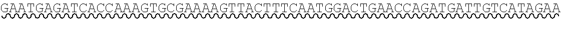

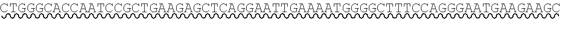

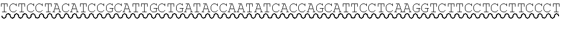

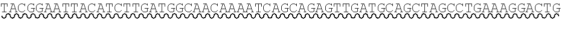

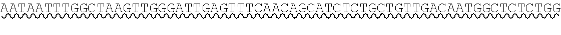

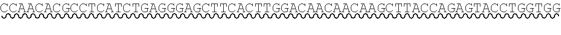

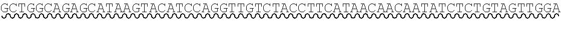

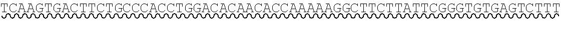

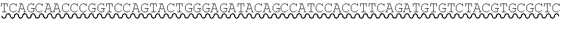

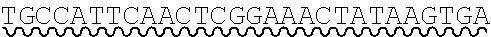

Signal peptide coding sequence is shown in boldface type. Heavy chain coding sequence is underlined with a straight line. Linker coding sequences are shown in italics. Galacorin coding sequences are underlined with a wavy line.

Avelumab-Galacorin2x Fusion Heavy Chain Protein Sequence
(SEQ ID NO: 55):
MMSFVSLLLVGILFHATQAEVQLLESGGGLVQPGGSLRLSCAASGFTFSSYIMMWVRQAPGKGL
EWVSSIYPSGGITFYADTVKGRFTISRDNSKNTLYLQMNSLRAEDTAVYYCARIKLGTVTTVDY
WGQGTLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNVFSCS

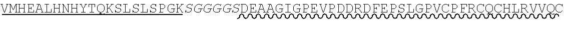

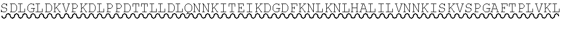

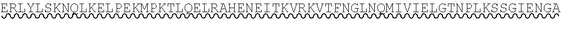

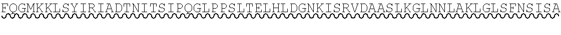

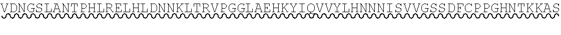

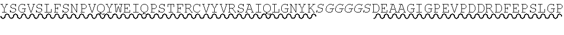

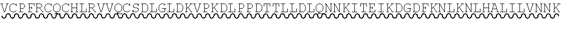

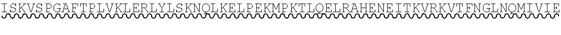

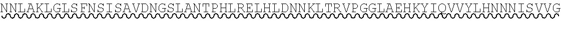

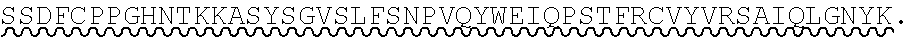

Signal peptide sequence is shown in boldface type. Heavy chain sequence is underlined with a straight line. Linker sequences are shown in italics. Galacorin sequences are underlined with a wavy line.

Avelumab-Galacorin/Decorin (Asp45-Lys359 of full length endogenous human Decorin) TGF-
Beta Binding Domains Fusion Heavy Chain Gene Sequence
(SEQ ID NO: 56):
ATGATGTCCTTTGTCTCTCTGCTCCTGGTTGGCATCCTATTCCATGCCACCCAGGCCGAGGTAC
AGCTTTTGGAGTCAGGCGGGGGGCTCGTCCAACCTGGGGGGTCACTCCGGTTGTCATGTGCTGC
CAGTGGCTTCACATTCTCATCTTACATTATGATGTGGGTTCGACAGGCCCCTGGGAAGGGGCTG
GAGTGGGTTTCCTCCATCTACCCCTCCGGGGGAATTACCTTCTATGCCGACACGGTAAAGGGTC
GCTTCACTATAAGTCGAGATAACAGCAAAAATACGCTGTATCTCCAGATGAACTCTCTCAGGGC
TGAGGACACAGCTGTATATTACTGCGCGCGGATTAAGTTGGGGACCGTCACAACAGTGGATTAC
TGGGGTCAAGGCACTCTGGTAACCGTATCCTCAGCATCCACCAAGGGCCCAAGTGTATTCCCGC
TGGCCCCTTCAAGTAAATCCACGTCTGGCGGCACAGCCGCTCTCGGTTGCCTGGTTAAGGACTA
CTTCCCAGAACCTGTCACTGTCAGTTGGAACTCAGGCGCATTGACATCTGGTGTCCATACATTC
CCCGCAGTCCTGCAAAGCTCTGGACTTTACAGTCTTAGTAGCGTAGTGACAGTCCCATCTTCAA
GTCTTGGGACCCAAACTTATATTTGCAACGTAAATCATAAACCCTCCAACACTAAAGTAGACAA
AAAAGTAGAGCCGAAATCTIGCGACAAAACGCATACATGCCCACCATGTCCCGCTCCGGAACTC
CTGGGCGGCCCGTCCGTTTTTCTCTTTCCCCCAAAGCCCAAGGATACGCTTATGATCAGCAGAA
CACCGGAAGTTACTTGTGTAGTCGTTGACGTGTCTCACGAAGATCCCGAAGTCAAATTTAATTG
GTATGTGGATGGCGTCGAAGTGCACAACGCAAAAACCAAACCCAGAGAGGAACAGTATAACAGC
ACGTATCGAGTGGTCTCCGTACTTACGGTCCTCCACCAGGACTGGTTGAATGGCAAGGAGTACA
AGTGCAAAGTGAGCAATAAAGCGTTGCCAGCCCCGATCGAAAAAACCATCAGCAAGGCCAAGGG
ACAGCCTAGAGAGCCGCAGGTTTACACCTTGCCGCCATCAAGGGATGAACTGACTAAAAACCAG
GTATCCCTGACCTGCCTGGTTAAGGGTTTTTACCCCAGTGATATAGCGGTTGAATGGGAGTCTA
ACGGGCAGCCAGAGAACAACTACAAAACGACACCTCCCGTTCTGGATTCCGATGGCAGCTTTTT
CTTGTATTCTAAACTCACCGTGGATAAATCCCGATGGCAGCAAGGCAACGTCTTCTCCTGCAGC
GTGATGCATGAAGCCTTGCACAACCACTATACCCAAAAGAGTCTCAGCCTGTCACCCGGGAAAT

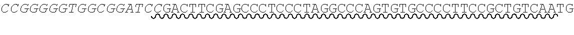

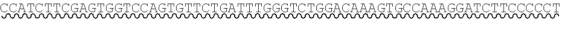

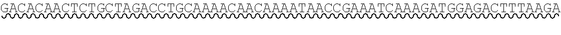

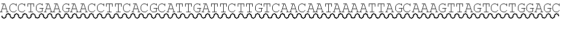

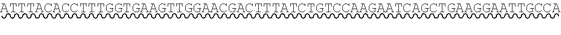

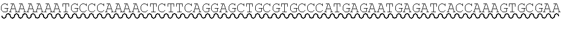

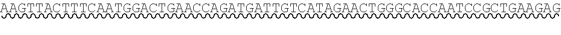

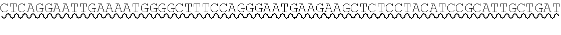

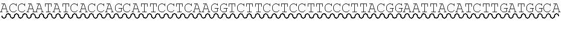

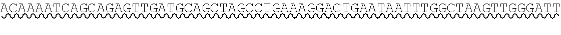

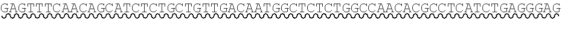

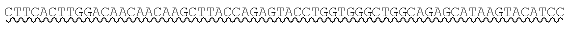

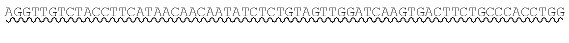

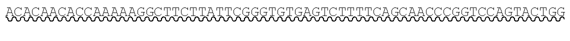

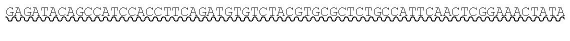

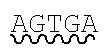

Signal peptide coding sequence is shown in boldface type. Heavy chain coding sequence is underlined with a straight line. Linker coding sequence is shown in italics. Galacorin/Decorin (Asp45-Lys 359) coding sequence is underlined with a wavy line.

Avelumab-Galacorin/Decorin (Asp45-Lys359 of full length endogenous human Decorin) TGF-
Beta Binding Domains Fusion Heavy Chain Protein Sequence
(SEQ ID NO: 57):
MMSFVSLLLVGILFHATQAEVQLLESGGGLVQPGGSLRLSCAASGFITSSYIMMWVRQAPGKGL
EWVSSIYPSGGITFYADTVKGRFTISRDNSKNTLYLQMNSLRAEDTAVYYCARIKLGTVTTVDY
WGQGTLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNVFSCS

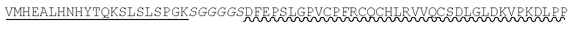

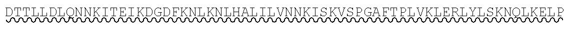

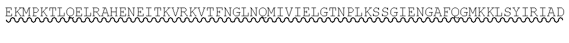

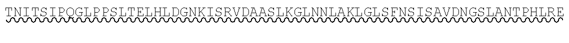

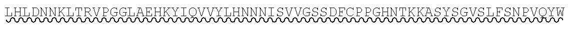

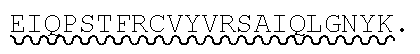

Signal peptide sequence is shown in boldface type. Heavy chain sequence is underlined with a straight line. Linker sequence is shown in italics. Galacorin/Decorin (Asp45-Lys 359) sequence is underlined with a wavy line.

Avelumab-Galacorin/Decorin (Asp45-Lys359 of full length endogenous human Decorin) TGF-
Beta Binding Domains 2x Fusion Heavy Chain Gene Sequence
(SEQ ID NO: 58):
ATGATGTCCTTTGTCTCTCTGCTCCTGGTTGGCATCCTATTCCATGCCACCCAGGCCGAGGTAC
AGCTTTTGGAGTCAGGCGGGGGGCTCGTCCAACCTGGGGGGTCACTCCGGTTGTCATGTGCTGC
CAGTGGCTTCACATTCTCATCTTACATTATGATGTGGGTTCGACAGGCCCCTGGGAAGGGGCTG
GAGTGGGTTTCCTCCATCTACCCCTCCGGGGGAATTACCTTCTATGCCGACACGGTAAAGGGTC
GCTTCACTATAAGTCGAGATAACAGCAAAAATACGCTGTATCTCCAGATGAACTCTCTCAGGGC
TGAGGACACAGCTGTATATTACTGCGCGCGGATTAAGTTGGGGACCGTCACAACAGTGGATTAC
TGGGGTCAAGGCACTCTGGTAACCGTATCCTCAGCATCCACCAAGGGCCCAAGTGTATTCCCGC
TGGCCCCTTCAAGTAAATCCACGTCTGGCGGCACAGCCGCTCTCGGTTGCCTGGTTAAGGACTA
CTTCCCAGAACCTGTCACTGTCAGTTGGAACTCAGGCGCATTGACATCTGGTGTCCATACATTC
CCCGCAGTCCTGCAAAGCTCTGGACTTTACAGTCTTAGTAGCGTAGTGACAGTCCCATCTTCAA
GTCTTGGGACCCAAACTTATATTTGCAACGTAAATCATAAACCCTCCAACACTAAAGTAGACAA
AAAAGTAGAGCCGAAATCTIGCGACAAAACGCATACATGCCCACCATGTCCCGCTCCGGAACTC
CTGGGCGGCCCGTCCGTTTTTCTCTTTCCCCCAAAGCCCAAGGATACGCTTATGATCAGCAGAA
CACCGGAAGTTACTTGTGTAGTCGTTGACGTGTCTCACGAAGATCCCGAAGTCAAATTTAATTG
GTATGTGGATGGCGTCGAAGTGCACAACGCAAAAACCAAACCCAGAGAGGAACAGTATAACAGC
ACGTATCGAGTGGTCTCCGTACTTACGGTCCTCCACCAGGACTGGTTGAATGGCAAGGAGTACA
AGTGCAAAGTGAGCAATAAAGCGTTGCCAGCCCCGATCGAAAAAACCATCAGCAAGGCCAAGGG
ACAGCCTAGAGAGCCGCAGGTTTACACCTTGCCGCCATCAAGGGATGAACTGACTAAAAACCAG
GTATCCCTGACCTGCCTGGTTAAGGGTTTTTACCCCAGTGATATAGCGGTTGAATGGGAGTCTA
ACGGGCAGCCAGAGAACAACTACAAAACGACACCTCCCGTTCTGGATTCCGATGGCAGCTTTTT
CTTGTATTCTAAACTCACCGTGGATAAATCCCGATGGCAGCAAGGCAACGTCTTCTCCTGCAGC
GTGATGCATGAAGCCTTGCACAACCACTATACCCAAAAGAGTCTCAGCCTGTCACCCGGGAAAT

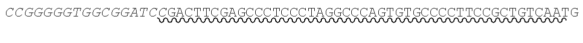

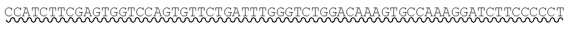

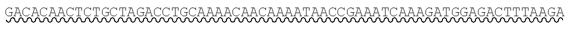

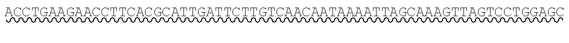

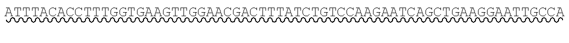

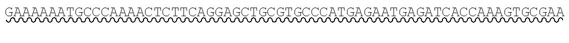

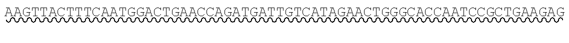

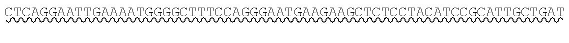

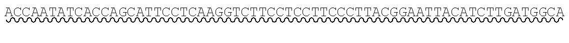

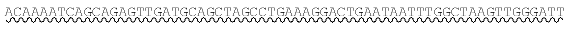

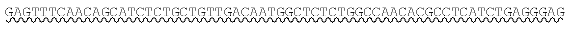

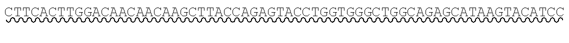

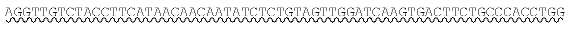

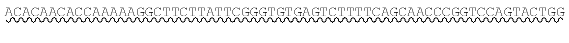

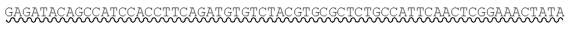

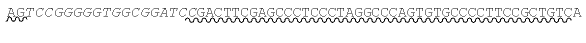

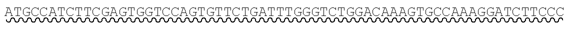

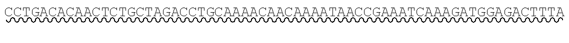

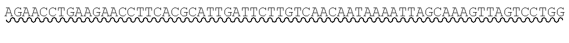

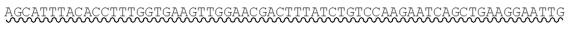

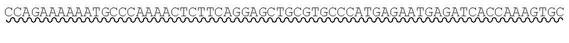

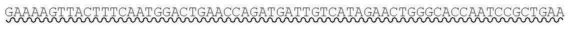

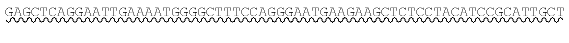

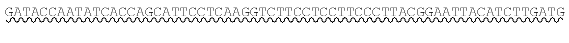

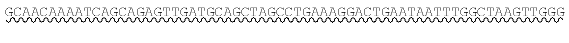

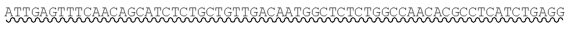

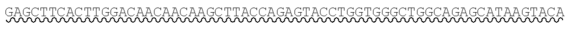

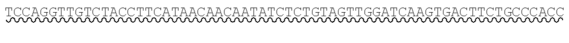

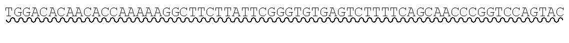

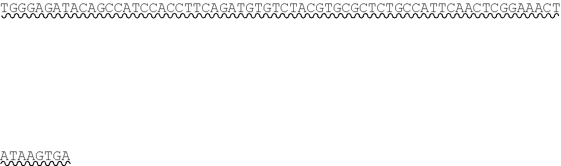

underlined with a straight line. Linker coding sequences are shown in italics. Galacorin/Decorin (Asp45-Lys 359) coding sequences are underlined with a wavy line.
Avelumab-Galacorin/Decorin (Asp45-Lys359 of full length endogenous human Decorin) TGF

Avelumab-Galacorin/Decorin (Asp45-Lys359 of full length endogenous human Decorin) TGF-
Beta Binding Domains 2x Fusion Heavy Chain Protein Sequence
(SEQ ID NO: 59):
MMSFVSLLLVGILFHATQAEVQLLESGGGLVQPGGSLRLSCAASGFTFSSYIMMWVRQAPGKGL
EWVSSIYPSGGITFYADTVKGRFTISRDNSKNTLYLQMNSLRAEDTAVYYCARIKLGTVTTVDY
WGQGTLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNVFSCS

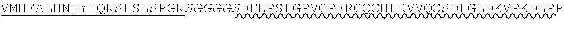

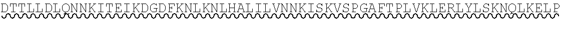

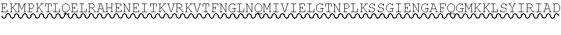

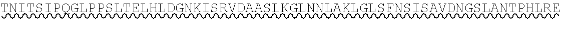

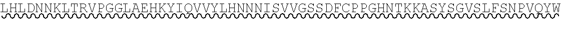

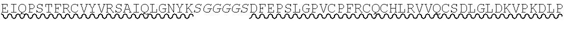

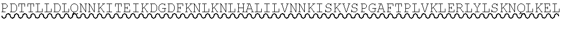

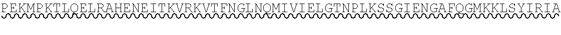

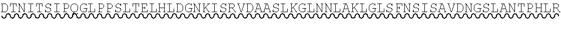

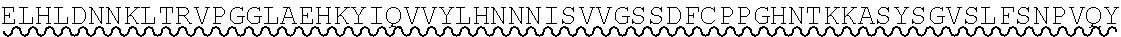

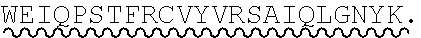

Signal peptide sequence is shown in boldface type. Heavy chain sequence is underlined with a straight line. Linker sequences are shown in italics. Galacorin/Decorin (Asp45-Lys 359) sequences are underlined with a wavy line.

Avelumab-Galacorin/Decorin (Leu155-Va1260 of full length endogenous human Decorin) TGF-
Beta Binding Domain Fusion Heavy Chain Gene Sequence
(SEQ ID NO: 60):
ATGATGTCCTTTGTCTCTCTGCTCCTGGTTGGCATCCTATTCCATGCCACCCAGGCCGAGGTAC
AGCTTTTGGAGTCAGGCGGGGGGCTCGTCCAACCTGGGGGGTCACTCCGGTTGTCATGTGCTGC
CAGTGGCTTCACATTCTCATCTTACATTATGATGTGGGTTCGACAGGCCCCTGGGAAGGGGCTG
GAGTGGGTTTCCTCCATCTACCCCTCCGGGGGAATTACCTTCTATGCCGACACGGTAAAGGGTC
GCTTCACTATAAGTCGAGATAACAGCAAAAATACGCTGTATCTCCAGATGAACTCTCTCAGGGC
TGAGGACACAGCTGTATATTACTGCGCGCGGATTAAGTTGGGGACCGTCACAACAGTGGATTAC
TGGGGTCAAGGCACTCTGGTAACCGTATCCTCAGCATCCACCAAGGGCCCAAGTGTATTCCCGC
TGGCCCCTTCAAGTAAATCCACGTCTGGCGGCACAGCCGCTCTCGGTTGCCTGGTTAAGGACTA
CTTCCCAGAACCTGTCACTGTCAGTTGGAACTCAGGCGCATTGACATCTGGTGTCCATACATTC
CCCGCAGTCCTGCAAAGCTCTGGACTTTACAGTCTTAGTAGCGTAGTGACAGTCCCATCTTCAA
GTCTTGGGACCCAAACTTATATTTGCAACGTAAATCATAAACCCTCCAACACTAAAGTAGACAA
AAAAGTAGAGCCGAAATCTIGCGACAAAACGCATACATGCCCACCATGTCCCGCTCCGGAACTC
CTGGGCGGCCCGTCCGTTTTTCTCTTTCCCCCAAAGCCCAAGGATACGCTTATGATCAGCAGAA
CACCGGAAGTTACTTGTGTAGTCGTTGACGTGTCTCACGAAGATCCCGAAGTCAAATTTAATTG
GTATGTGGATGGCGTCGAAGTGCACAACGCAAAAACCAAACCCAGAGAGGAACAGTATAACAGC
ACGTATCGAGTGGTCTCCGTACTTACGGTCCTCCACCAGGACTGGTTGAATGGCAAGGAGTACA
AGTGCAAAGTGAGCAATAAAGCGTTGCCAGCCCCGATCGAAAAAACCATCAGCAAGGCCAAGGG
ACAGCCTAGAGAGCCGCAGGTTTACACCTTGCCGCCATCAAGGGATGAACTGACTAAAAACCAG
GTATCCCTGACCTGCCTGGTTAAGGGTTTTTACCCCAGTGATATAGCGGTTGAATGGGAGTCTA
ACGGGCAGCCAGAGAACAACTACAAAACGACACCTCCCGTTCTGGATTCCGATGGCAGCTTTTT
CTTGTATTCTAAACTCACCGTGGATAAATCCCGATGGCAGCAAGGCAACGTCTTCTCCTGCAGC
GTGATGCATGAAGCCTTGCACAACCACTATACCCAAAAGAGTCTCAGCCTGTCACCCGGGAAAT

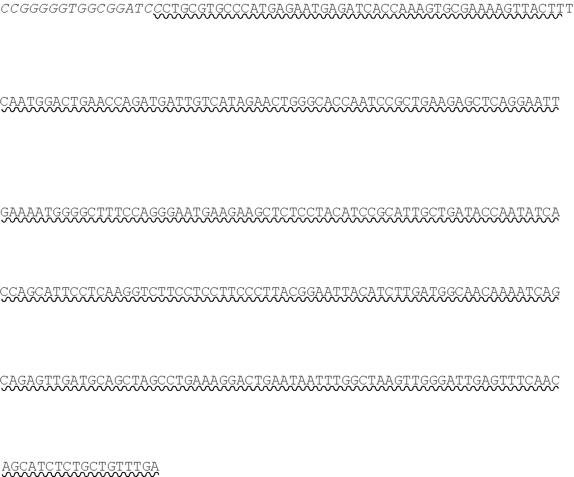

Signal peptide coding sequence is shown in boldface type. Heavy chain coding sequence is underlined with a straight line. Linker coding sequence is shown in italics. Galacorin/Decorin (Leu155-Va1260) coding sequence is underlined with a wavy line.

Avelumab-Galacorin/Decorin (Leu155-Va1260 of full length endogenous human Decorin) TGF-
Beta Binding Domain Fusion Heavy Chain Protein Sequence
(SEQ ID NO: 61):
MMSFVSLLLVGILFHATQAEVQLLESGGGLVQPGGSLRLSCAASGFITSSYIMMWVRQAPGKGL
EWVSSIYPSGGITFYADTVKGRFTISRDNSKNTLYLQMNSLRAEDTAVYYCARIKLGTVTTVDY
WGQGTLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNVFSCS

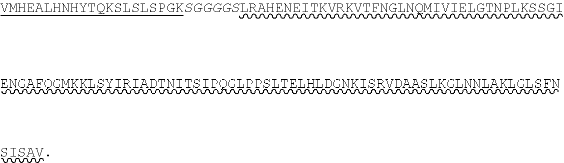

Signal peptide sequence is shown in boldface type. Heavy chain sequence is underlined with a straight line. Linker sequence is shown in italics. Galacorin/Decorin (Leu155-Va1260) sequence is underlined with a wavy line.

Avelumab-Galacorin/Decorin (Leu155-Va1260 of full length endogenous human Decorin) TGF-
Beta Binding Domain 2x Fusion Heavy Chain Gene Sequence
(SEQ ID NO: 62):
ATGATGTCCTTTGTCTCTCTGCTCCTGGTTGGCATCCTATTCCATGCCACCCAGGCCGAGGTAC
AGCTTTTGGAGTCAGGCGGGGGGCTCGTCCAACCTGGGGGGTCACTCCGGTTGTCATGTGCTGC
CAGTGGCTTCACATTCTCATCTTACATTATGATGTGGGTTCGACAGGCCCCTGGGAAGGGGCTG
GAGTGGGTTTCCTCCATCTACCCCTCCGGGGGAATTACCTTCTATGCCGACACGGTAAAGGGTC
GCTTCACTATAAGTCGAGATAACAGCAAAAATACGCTGTATCTCCAGATGAACTCTCTCAGGGC
TGAGGACACAGCTGTATATTACTGCGCGCGGATTAAGTTGGGGACCGTCACAACAGTGGATTAC
TGGGGTCAAGGCACTCTGGTAACCGTATCCTCAGCATCCACCAAGGGCCCAAGTGTATTCCCGC
TGGCCCCTTCAAGTAAATCCACGTCTGGCGGCACAGCCGCTCTCGGTTGCCTGGTTAAGGACTA
CTTCCCAGAACCTGTCACTGTCAGTTGGAACTCAGGCGCATTGACATCTGGTGTCCATACATTC
CCCGCAGTCCTGCAAAGCTCTGGACTTTACAGTCTTAGTAGCGTAGTGACAGTCCCATCTTCAA
GTCTTGGGACCCAAACTTATATTTGCAACGTAAATCATAAACCCTCCAACACTAAAGTAGACAA
AAAAGTAGAGCCGAAATCTTGCGACAAAACGCATACATGCCCACCATGTCCCGCTCCGGAACTC
CTGGGCGGCCCGTCCGTTTTTCTCTTTCCCCCAAAGCCCAAGGATACGCTTATGATCAGCAGAA
CACCGGAAGTTACTTGTGTAGTCGTTGACGTGTCTCACGAAGATCCCGAAGTCAAATTTAATTG
GTATGTGGATGGCGTCGAAGTGCACAACGCAAAAACCAAACCCAGAGAGGAACAGTATAACAGC
ACGTATCGAGTGGTCTCCGTACTTACGGTCCTCCACCAGGACTGGTTGAATGGCAAGGAGTACA
AGTGCAAAGTGAGCAATAAAGCGTTGCCAGCCCCGATCGAAAAAACCATCAGCAAGGCCAAGGG
ACAGCCTAGAGAGCCGCAGGTTTACACCTTGCCGCCATCAAGGGATGAACTGACTAAAAACCAG
GTATCCCTGACCTGCCTGGTTAAGGGTTTTTACCCCAGTGATATAGCGGTTGAATGGGAGTCTA
ACGGGCAGCCAGAGAACAACTACAAAACGACACCTCCCGTTCTGGATTCCGATGGCAGCTTTTT
CTTGTATTCTAAACTCACCGTGGATAAATCCCGATGGCAGCAAGGCAACGTCTTCTCCTGCAGC
GTGATGCATGAAGCCTTGCACAACCACTATACCCAAAAGAGTCTCAGCCTGTCACCCGGGAAAT

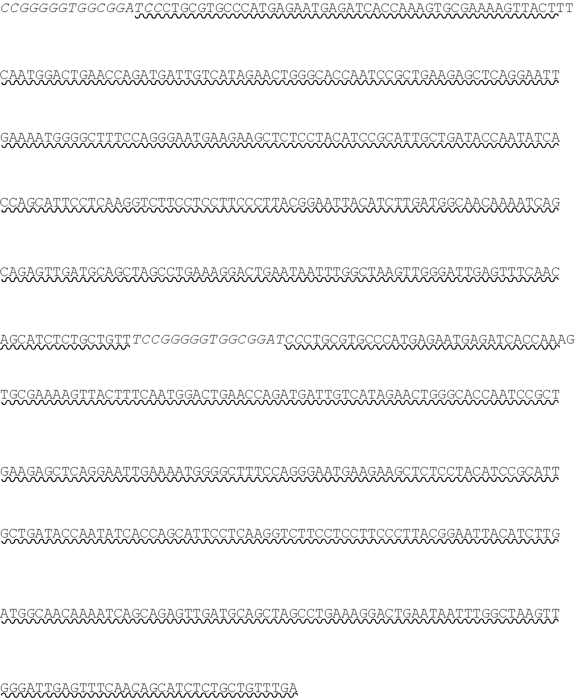

Signal peptide coding sequence is shown in boldface type. Heavy chain coding sequence is underlined with a straight line. Linker coding sequences are shown in italics. Galacorin/Decorin (Leu155-Va1260) coding sequences are underlined with a wavy line.

Avelumab-Galacorin/Decorin (Leu155-Va1260 of full length endogenous human Decorin) TGF-
Beta Binding Domain 2x Fusion Heavy Chain Protein Sequence
(SEQ ID NO: 63):
MMSFVSLLLVGILFHATQAEVQLLESGGGLVQPGGSLRLSCAASGFTFSSYIMMWVRQAPGKGL
EWVSSIYPSGGITFYADTVKGRFTISRDNSKNTLYLQMNSLRAEDTAVYYCARIKLGTVTTVDY
WGQGTLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNVFSCS

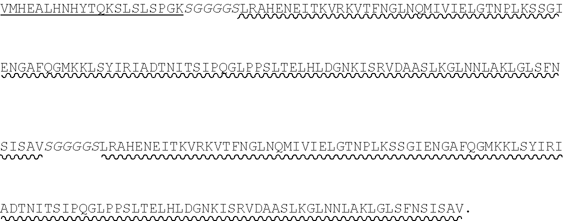

Signal peptide sequence is shown in boldface type. Heavy chain sequence is underlined with a straight line. Linker sequences are shown in italics. Galacorin/Decorin (Leu155-Val260) sequences are underlined with a wavy line.

### Example 2

This example describes the production of expression cells lines for the production of the fusion proteins described in Example 1.

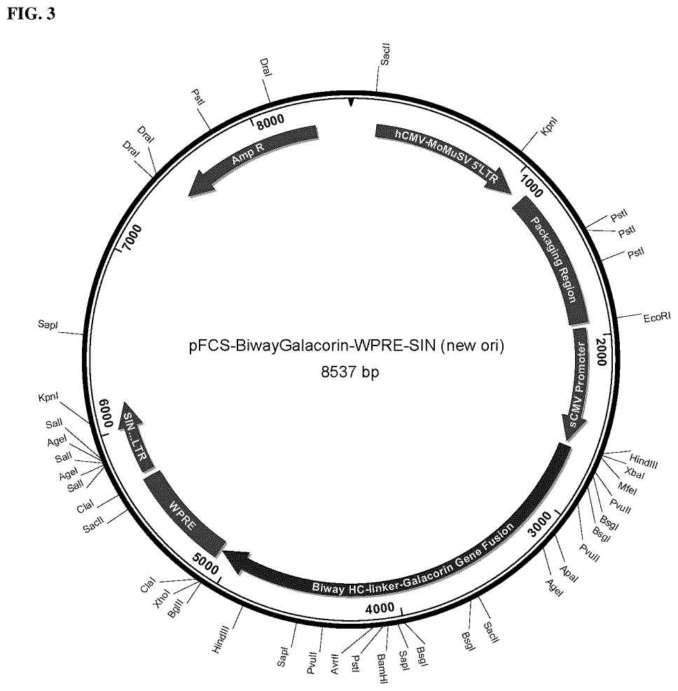

Retrovector Production: The expression constructs outlined above were introduced into a HEK 293 cell line that constitutively produces the MLV gag, pro, and pol proteins. An envelope containing expression plasmid was also co-tranfected with either the bevacizumab light chain (See FIG. 2 for vector map) or bevacizumab heavy chain-galacorin fusion (See FIG. 3 for vector map) gene construct. The two co-transfections resulted in the production of replication incompetent high titer retrovector for either light chain or heavy chain-galacorin, that was concentrated by ultracentrifugation and used for cell transductions (See, e.g., Bleck, G. T. 2005 An alternative method for the rapid generation of stable, high-expressing mammalian cell lines (A Technical Review). Bioprocessing J. Setp/Oct. pp 1-7; Bleck, G. T., 2010. GPEx® A Flexible Method for the Rapid Generation of Stable, High Expressing, Antibody Producing Mammalian Cell Lines Chapter 4 In: Current Trends in Monoclonal Antibody Development and Manufacturing, Biotechnology: Pharmaceutical Aspects, Edited by: S. J. Shire et al. © 2010 American Association of Pharmaceutical Scientists, DOI 10.1007/978-0-387-76643-O_4.).

Summary Features of pCS-Bi-wayLC-WPRE(new ori), GDD2107.0003

| Component | Description | Function/Notes |
|---|---|---|
| 5′ hCMV-MoMuSV LTR (R- | A fusion of the human | The human cytomegalovirus IE promoter has |
| U5) | CMV promoter to the | strong constitutive activity in most |
| bp 142-766 hCMV | R-U5 regions of the | mammalian cells. Used to create high titer of |
| promoter | Moloney Murine | retrovector particles when transfected into |
| bp 767-911 MoMuSV | Sarcoma Virus 5′ LTR | packaging cells. |
| R-U5 | | The hCMV promoter is lost after the |
| | | packaging cell step. |
| Extended packaging region | MoMuLV/SV | Packaging region allows creation of |
| bp 981-1790 | packaging region | retrovector particles by allowing RNA to |
| | from the LTR through | associate with MoMuLV Gag/Pol gene |
| | a mutated ATG site in | products. |
| | the MLV Gag gene | |
| sCMV promoter | The immediate early | Alternative strong constitutive promoter to |
| bp 1822-2424 | promoter from simian | drive expression of product gene. |
| | CMV | |
| Biway light chain gene | Biway light chain | Biway light chain CDS directly cloned from |
| bp 2510-3211 | CDS | Bio Basic's pUC57 plasmid |
| WPRE | A fragment from the | Region that is thought to aid export of |
| bp 3230-3830 | woodchuck hepatitis | unspliced RNA and improve protein |
| | B virus Pol gene | expression. |
| 3′ LTR | The 3′ LTR from | Functions as a Poly A signal for RNA. |
| bp 3870-4463 | MoMuLV | Allows reverse transcription and DNA |
| | | insertion of retrovector into mammalian cells |
| | | from retrovector particles. |
| Plasmid backbone - E. coli | Basic E. coli plasmid | Allows selection of plasmid containing |
| origin of replication and β- | sequences | bacteria in E. coli and replication of DNA in |
| lactamase gene for ampicillin | | E. coli. |
| resistance | | These regions are lost after transfecting |
| bp 5765 < 6625 | | plasmids into packaging cells and creating |
| bp 1-148 | | retrovector particles. |

Summary: Features of pFCS-BiwayGalacorin-WPRE-SIN (new ori), GDD2134.0001

| Component | Description | Function/Notes |
|---|---|---|
| 5′ hCMV-MoMuSV LTR | A fusion of the full- | The human cytomegalovirus IE promoter has |
| (R-U5) | length human CMV | strong constitutive activity in most |
| bp 149-865 hCMV | promoter to the | mammalian cells. Used to create high titer of |
| promoter | R-U5 regions of the | retrovector particles when transfected into |
| bp 866-1041 MoMuSV | Moloney Murine | packaging cells. |
| R-U5 | Sarcoma Virus 5′ LTR | The hCMV promoter is lost after the |
| | | packaging cell step. |
| Extended packaging region | MoMuLV/SV | Packaging region allows creation of |
| bp 1111-1920 | packaging region | retrovector particles by allowing RNA to |
| | from the LTR through | associate with MoMuLV Gag/Pol gene |
| | a mutated ATG site in | products. |
| | the MLV Gag | gene |
| sCMV promoter | The immediate early | Alternative strong constitutive promoter to |
| bp 1952-2624 | promoter from simian | drive expression of product gene. |
| | CMV | |
| Biway HC-Galacorin gene | Biway HC-Galacorin | Full Biway HC-linker-Galacorin CDS |
| fusion | CDS | assembled by PCR and cloned by restriction |
| bp 2650-5073 | | digestion |
| WPRE | A fragment from the | Region that is thought to aid export of |
| bp 5089-5689 | woodchuck Hepatitis | unspliced RNA and improve protein |
| | B virus Pol gene | expression. |
| SIN 3′ LTR | The 3′ LTR from | Functions as a Poly A signal for RNA. |
| bp 5730-6153 | MoMuLV | Allows reverse transcription and DNA |
| | | insertion of retrovector into mammalian cells |
| | | from retrovector particles. In proviral DNA |
| | | deletion in U3 region is duplicated to 5′ LTR |
| | | hereby inactivating 5′ LTR promoter activity. |
| Plasmid backbone - E. coli | Basic E. coli plasmid | Allows selection of plasmid containing |
| origin of replication and β- | sequences | bacteria in E. coli and replication of DNA in |
| lactamase gene for ampicillin | | E. coli. |
| resistance | | These regions are lost after transfecting |
| bp 7472-8332 | | plasmids into packaging cells and creating |
| bp 1-148 | | retrovector particles. |

Transduction of GCHO Cells with Retrovector: The bevacizumab-galacorin fusion antibody pooled cell line was produced by multiple cycles of cell transduction of the GPEx® Chinese Hamster Ovary (GCHO) parental cell line, with two light chain transductions followed by two heavy chain-galacorin transductions performed once a week over the span of 4 weeks. These transductions were performed to generate a pooled cell line each of the two gene products.

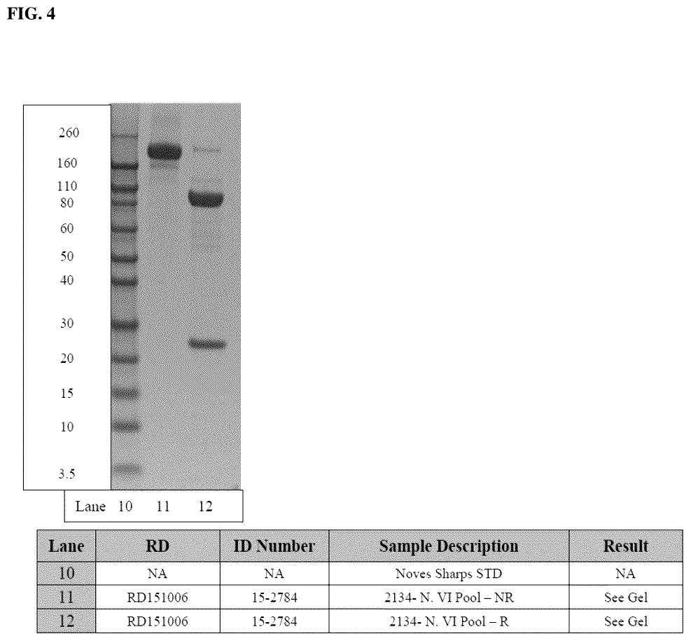

Fed Batch Production of Bevacizumab-Galacorin Fusion from the Pooled Population of Cells: Post-transduction, the pooled cell line for the Bevacizumab-Galacorin Fusion was scaled up for productivity in a fed batch study in duplicate 250 mL shake flasks. Each shake flask was seeded with 300,000 viable cells per mL in a 60 mL working volume of PF CHO LS media (HyClone) and incubated in a humidified (70-80%) shaking incubator at 130 rpm with 5% CO₂ and temperature of 37° C. Cultures were fed four times during the production run using two different feed supplements. Cultures were terminated when viabilities were <70%. Confirmation of the fusion antibody production was determined by SDS-PAGE gel analysis (FIG. 4) and by ELISA to quantitate the amount of product produced. The cultures produce 360 mg/L of the fusion antibody product. The product behave as expected in SDS-PAGE showing a predominant single band under non-reduced conditions and two bands (Heavy chain-galacorin fusion and light chain) under reduced conditions. The −80 kDa size of the fusion heavy chain matches the expected size of that product and the light chain is the normal 25 kDa in size.

### Example 3

This example describes expression of an Avelumab-Galacorin fusion.

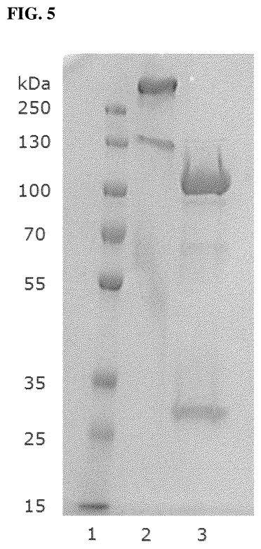

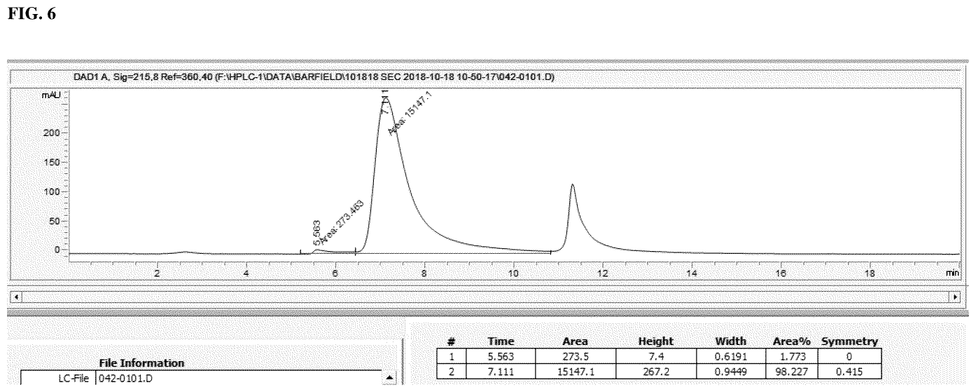

The gene construct of SEQ ID NO:50 was transiently transfected into ExpiCHO cells and production of the fusion molecule was performed at the 250 ml scale. The titer of the fusion on day of harvest was 377 mg/L. The fusion was purified over a MabSelectSuRe protein A column. The resulting purified protein was buffer exchanged into 20 mM NaCitrate, pH 5.5, 50 mM NaCl using tangential flow filtration. The resulting material was examined using SDS-PAGE gels and SEC-HPLC. The product showed expected size profile on the SDS-PAGE gel (FIG. 5) and showed very low aggregation levels on SEC-HPLC (FIG. 6).

Material produced in the above production was used for evaluation in a mouse tumor model study. The study used C57BL/6 mice and the MC-38 human colorectal cancer cells. Forty C57BL/6 mice were injected subcutaneously with MC-38 cells. Tumors were allowed to grow to approximately 100 mm 3 and then 10 mice each were assigned to 4 different treatments. Each group received a single dose treatment IV and tumor size was measure over subsequent days. Treatment groups were Vehicle, Galacorin/Decorin 4 mg/kg, Avelumab/Anti-PD-L1 17 mg/kg, or Avelumab/Anti PD-L1-Decorin/Galacorin fusion 25 mg/kg.

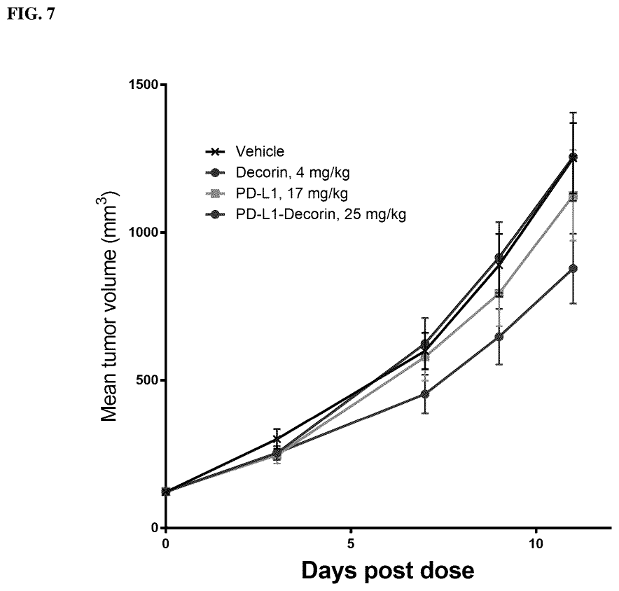

Tumor growth was inhibited by both the Avelumab/Anti-PD-L1 and Avelumab/Anti PD-L1-Decorin/Galacorin fusion treatments as compared to Vehicle and Decorin/Galacorin alone (FIG. 7). In addition, Avelumab/Anti PD-L1-Decorin/Galacorin fusion treatment also inhibited growth more effectively than Avelumab/Anti-PD-L1 alone.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of this invention are intended to be within the scope of the following claims.

What is claimed is:

1. A method of inhibiting a target protein and a signaling molecule in a cell, comprising:
contacting the cell with the multifunctional protein molecule of claim 1 under conditions such that at least one activity of the target protein and at least one activity of a signaling protein are inhibited in the cell, wherein the target protein is selected from the group consisting of VEGF-1, PD-1, PD-L1, CTLA-4, PD-L2, CD27, CD28, CD40, CD47, CD115, CD122, CD137, OX40, GITR, ICOS, A2AR, B7-H3, B7-H4, BTLA, IDO, KIR, LAG3, NOX2, TIM-3, VISTA, SIGLEC-7, TIGIT and 4-1BB.


SEQUENCE LISTING (67 sequences)

SEQ ID NO: 1 - DNA, 2424 bp, synthetic construct
  source 1..2424: mol_type = other DNA
atgatgtcctttgtctctctgctcctggttggcatcctattccatgccacccaggccgaggtgcagctggtggagtctgggggaggcttggtacagcctggggggtccctgagactctcctgtgcagcctctggatacacctttaccaactatggcatgaactgggtccgccaggctccagggaaggggctggagtgggtgggctggataaacacttacactggtgagccaacatatgcagctgacttcaagcgccggtttaccttctctttggacacctccaagtccacggcctatctgcaaatgaacagcctgcgggccgaggacacggccgtatattactgtgcgaaatacccccactactacggtagtagccactggtactttgacgtgtggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatccgggggtggcggatccgatgaggctgcagggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtga

SEQ ID NO: 2 - protein, 807 aa, synthetic construct
MMSFVSLLLVGILFHATQAEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSDEAAGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 3 - DNA, 702 bp, synthetic construct
  source 1..702: mol_type = other DNA
atgatgtcctttgtctctctgctcctggttggcatcctgttccatgccacccaggccgacatccagatgacccagtctccatcctccctgtctgcatctgtaggagacagagtcaccatcacttgcagtgcaagtcaggacattagcaattatttaaactggtatcagcagaaaccagggaaagctcctaaggtcctgatctatttcacatccagtttgcactcaggggtcccatctaggttcagtggcagtggatctgggacagatttcactctcaccatcagcagcctgcagcctgaagattttgcaacttattactgccaacagtatagtaccgtgccttggacgttcggccaagggaccaaggtggaaatcaaacgaactgtggctgcaccatctgtcttcatcttcccgccatctgatgagcagcttaagtccggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 4 - protein, 233 aa, synthetic construct
MMSFVSLLLVGILFHATQADIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 19 aa, synthetic construct
MMSFVSLLLVGILFHATQA

SEQ ID NO: 6 - protein, 6 aa, synthetic construct
SGGGGS

SEQ ID NO: 7 - protein, 329 aa, synthetic construct
DEAAGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 8 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 214 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 2409 bp, synthetic construct
  source 1..2409: mol_type = other DNA
atgatgtcctttgtctctctgctcctggttggcatcctattccatgccacccaggcccaggtgcagctggtggagtccggcggcggcgtcgtgcagcccggccggtccctgcggctgtcctgcgccgcctccggcttcaccttctcctcctacaccatgcactgggtgcggcaggcccccggcaagggcctggagtgggtgactttcatctcctacgacggcaacaacaagtactacgccgactccgtgaagggccggttcaccatctcccgcgacaactccaagaacaccctgtacctgcagatgaactccctgcgggccgaggacaccgccatctactactgcgcccggaccggctggctgggccccttcgactactggggccagggcaccctggtgaccgtgtcctccgcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagcgggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcctgggaaatccgggggtggcggatccgatgaggctgcagggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtga

SEQ ID NO: 11 - protein, 802 aa, synthetic construct
MMSFVSLLLVGILFHATQAQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSDEAAGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 12 - DNA, 705 bp, synthetic construct
  source 1..705: mol_type = other DNA
atgatgtcctttgtctctctgctcctggttggcatcctattccatgccacccaggccgagatcgtgctgacccagtcccccggcaccctgtccctgtcccccggcgagcgggccaccctgtcctgccgggcctcccagtccgtgggctcctcctacctggcctggtaccagcagaagcccggccaggccccccggctgctgatctacggcgccttctcccgcgccaccggcatccccgaccggttctccggctccggctccggcaccgacttcaccctgaccatctcccggctggagcccgaggacttcgccgtgtactactgccagcagtacggctcctccccctggaccttcggccagggcaccaaggtggagatcaagcgaactgtggctgcaccatctgtcttcatcttcccgccatctgatgagcagcttaagtccggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 13 - protein, 234 aa, synthetic construct
MMSFVSLLLVGILFHATQAEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 448 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 215 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 2409 bp, synthetic construct
  source 1..2409: mol_type = other DNA
atgatgtcctttgtctctctgctcctggttggcatcctattccatgccacccaggccgaggtacaactcgtggaatccggcgggggactcgtccaaccgggtggtagtctcaggttgagctgcgctgcgagcggtttcactttctcagactcatggattcattgggtgcgccaagcacctggaaaagggttggaatgggttgcctggatctctccttacggaggttctacctactacgctgattcagtaaaggggcggttcacaatttcagccgatacttccaaaaatacggcttacctgcaaatgaactctttgagggcggaggatacggcggtctactactgtgcacgcaggcactggcctggagggttcgattattggggtcaaggcactttggtaaccgtatcctccgcttctaccaaaggcccatcagtatttcctttggctcccagctctaagtccacttccggtggaactgccgcacttggatgtctcgtcaaagactactttcctgagccggtaactgtgtcatggaactccggcgccctcactagcggcgtccatacatttccagcggttctccagtcaagtggcctctacagcctgtccagtgtagttactgtcccgtcttctagtctgggaacgcaaacatatatttgcaatgtgaatcataagcctagtaacacaaaagtcgataaaaaagtggagccgaaaagttgtgacaaaacgcatacctgtccgccttgtccggcccccgaactcttgggcggcccatcagtctttctcttcccgcccaaacctaaggacacgttgatgataagtcgcacgcccgaggttacatgcgtcgtagtcgatgtcagccacgaggatccggaggtaaagtttaactggtatgtagacggagttgaagtacacaacgccaaaactaaaccgagagaggagcagtacgcatcaacctatcgcgtagtatctgtattgacggtccttcaccaagactggctcaatgggaaagaatacaagtgcaaagtttctaataaagccctccctgcaccaatcgaaaagactatttcaaaagccaaaggacaaccaagagaaccacaagtttatacattgccacctagtcgcgaggagatgactaaaaaccaagtgtcccttacttgtctcgtaaagggtttctatccaagcgacatagcagttgagtgggaaagtaatggccagccggaaaacaactacaagacgacccccccggttctcgactccgatggatcattctttttgtatagtaaactcacagttgataagagtcgatggcagcaggggaatgttttttcttgctctgtgatgcacgaggcgctccacaaccactatacgcaaaagtccctcagcctgagccccgggaaatccgggggtggcggatccgatgaggctgcagggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtga

SEQ ID NO: 17 - protein, 802 aa, synthetic construct
MMSFVSLLLVGILFHATQAEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSDEAAGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 18 - DNA, 702 bp, synthetic construct
  source 1..702: mol_type = other DNA
atgatgtcctttgtctctctgctcctggttggcatcctattccatgccacccaggccgacattcagatgacacaatcacctagcagtctgagtgcgagcgtaggtgatcgcgtaacgattacctgcagggcctctcaagacgtgtcaacggcagtggcgtggtaccagcagaagcctggtaaagctcctaagctcctcatctattcagcttccttcttgtatagtggagtaccgtcaagattttccggaagcggatcaggtacagattttactttgactatcagtagtttgcagccagaggatttcgctacatattactgtcaacaatatctctatcaccctgccacttttggacaagggactaaagtcgaaataaaacgaacagtggccgcaccaagcgtttttatctttcccccatccgacgagcagttgaagagcggcaccgcgtccgtggtctgcctgttgaataatttctatccaagggaggcaaaagtgcaatggaaagttgataatgcgcttcaatccggaaactcacaagaatcagtaacagaacaagactctaaagacagtacatattctcttagtagcacactcactctttcaaaggctgactatgagaaacataaagtgtacgcttgtgaagtgacacatcaaggtcttagctccccagtaactaagagctttaataggggcgagtgctga

SEQ ID NO: 19 - protein, 233 aa, synthetic construct
MMSFVSLLLVGILFHATQADIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - DNA, 2415 bp, synthetic construct
  source 1..2415: mol_type = other DNA
atgatgtcctttgtctctctgctcctggttggcatcctattccatgccacccaggccgaggtacagcttttggagtcaggcggggggctcgtccaacctggggggtcactccggttgtcatgtgctgccagtggcttcacattctcatcttacattatgatgtgggttcgacaggcccctgggaaggggctggagtgggtttcctccatctacccctccgggggaattaccttctatgccgacacggtaaagggtcgcttcactataagtcgagataacagcaaaaatacgctgtatctccagatgaactctctcagggctgaggacacagctgtatattactgcgcgcggattaagttggggaccgtcacaacagtggattactggggtcaaggcactctggtaaccgtatcctcagcatccaccaagggcccaagtgtattcccgctggccccttcaagtaaatccacgtctggcggcacagccgctctcggttgcctggttaaggactacttcccagaacctgtcactgtcagttggaactcaggcgcattgacatctggtgtccatacattccccgcagtcctgcaaagctctggactttacagtcttagtagcgtagtgacagtcccatcttcaagtcttgggacccaaacttatatttgcaacgtaaatcataaaccctccaacactaaagtagacaaaaaagtagagccgaaatcttgcgacaaaacgcatacatgcccaccatgtcccgctccggaactcctgggcggcccgtccgtttttctctttcccccaaagcccaaggatacgcttatgatcagcagaacaccggaagttacttgtgtagtcgttgacgtgtctcacgaagatcccgaagtcaaatttaattggtatgtggatggcgtcgaagtgcacaacgcaaaaaccaaacccagagaggaacagtataacagcacgtatcgagtggtctccgtacttacggtcctccaccaggactggttgaatggcaaggagtacaagtgcaaagtgagcaataaagcgttgccagccccgatcgaaaaaaccatcagcaaggccaagggacagcctagagagccgcaggtttacaccttgccgccatcaagggatgaactgactaaaaaccaggtatccctgacctgcctggttaagggtttttaccccagtgatatagcggttgaatgggagtctaacgggcagccagagaacaactacaaaacgacacctcccgttctggattccgatggcagctttttcttgtattctaaactcaccgtggataaatcccgatggcagcaaggcaacgtcttctcctgcagcgtgatgcatgaagccttgcacaaccactatacccaaaagagtctcagcctgtcacccgggaaatccgggggtggcggatccgatgaggctgcagggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtga

SEQ ID NO: 23 - protein, 804 aa, synthetic construct
MMSFVSLLLVGILFHATQAEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSDEAAGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 24 - DNA, 708 bp, synthetic construct
  source 1..708: mol_type = other DNA
atgatgtcctttgtctctctgctcctggttggcatcctattccatgccacccaggcccagtctgcacttacacaaccggcgtccgtttccggatctccaggacagagcattactatcagttgcacgggaacctcctcagacgtaggggggtataattatgtgtcttggtatcaacagcatcccgggaaagcccccaaactgatgatctacgatgtcagcaatagaccaagcggtgtgagtaatcgatttagcgggtctaaatctggtaacacagcatccctcactattagtggactgcaagcagaagatgaggcagactattattgcagtagctatacgtctagttccacccgcgtttttggcactgggacgaaagtcaccgttctcggacaaccaaaagcaaaccccaccgtgactctgtttccgcctagcagcgaagaattgcaggccaataaggcgacactcgtatgccttatctccgacttctacccgggcgctgtgacagtcgcgtggaaagccgacggcagccctgttaaagctggagtcgagaccacgaagccgtccaagcagagtaacaataagtatgctgcatccagttatctctctctcactccggaacagtggaagtcccatcggtcctatagttgccaagtgacccatgagggttccaccgtagagaaaacggtagcacctaccgaatgtagttga

SEQ ID NO: 25 - protein, 235 aa, synthetic construct
MMSFVSLLLVGILFHATQAQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 26 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 28 - DNA, 2418 bp, synthetic construct
  source 1..2418: mol_type = other DNA
atgatgtcctttgtctctctgctcctggttggcatcctattccatgccacccaggccgaggtccagcttgttgaaagcggtggtggcctcgtgcagcctggtggcagtttgcggttgtcttgcgcagctagtggttttaccttctccagatactggatgtcatgggtccgacaggcccctggcaaggggcttgaatgggttgcaaatataaagcaggacggttctgaaaagtactatgtagactccgtcaaaggaagatttactattagtcgagacaacgccaagaatagcctctaccttcagatgaattctttgcgagcggaggacacagccgtatattactgcgcacgagaaggggggtggttcggtgaactggcttttgactactgggggcaaggtacgcttgtcacggtgagctctgcctctacaaaggggccgtctgtgtttccacttgctccatctagtaagtcaacttctggaggtactgcggcattgggatgccttgttaaggattattttcccgaacctgtaactgtgagctggaattcaggtgccctcacctctggtgtacatacctttccagcagttttgcaatcttccggtttgtactctcttagttcagttgtaactgtcccctcttcctctcttggtacccaaacatacatttgtaatgtcaatcacaaaccaagcaataccaaggtagacaagcgggtggaacccaaatcttgtgacaaaactcatacctgcccaccatgtcccgccccggagtttgaaggaggtccaagtgtatttcttttcccgcctaagcctaaggataccctcatgataagtcggacaccagaggtgacgtgtgttgtggtagacgtgagtcacgaagatcccgaagttaaatttaattggtatgtggacggggtggaagtccataacgcgaagacaaagccacgcgaagagcagtacaattccacgtacagggtggttagcgtgcttaccgtcctgcatcaagattggctgaacgggaaagaatacaaatgcaaagtatccaacaaggcgttgcctgcgagtatcgagaaaacgatttctaaagctaaaggacaaccccgggaaccccaggtctatacactgccgcccagcagagaagagatgacgaaaaatcaagtatcccttacgtgtctcgtcaaaggcttctacccttccgatattgctgtagagtgggaatctaacgggcagccggaaaataactacaagactactccgccagtacttgattcagacggctccttcttcctttattcaaaactcaccgtagataaaagtaggtggcaacaaggtaatgtttttagctgtagcgtaatgcacgaagcgttgcataaccattatacacagaaatcactcagcctgtcccccgggaaatccgggggtggcggatccgatgaggctgcagggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtga

SEQ ID NO: 29 - protein, 805 aa, synthetic construct
MMSFVSLLLVGILFHATQAEVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSDEAAGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 30 - DNA, 705 bp, synthetic construct
  source 1..705: mol_type = other DNA
atgatgtcctttgtctctctgctcctggttggcatcctattccatgccacccaggccgagatagttttgactcaaagccctggaacgctctctttgtctcccggcgagcgggcgaccctttcctgtagggctagccagagggtatcttcctcttacctggcatggtaccagcaaaagcccggacaagccccccgacttctgatttatgacgcctcatcccgggcgacaggcatccctgaccgattttcagggagtggctctggtaccgattttacgcttacgatttccaggctggagcccgaggatttcgcagtgtattactgtcaacaatacggcagcttgccctggacctttggacaaggaaccaaggtagagatcaaaaggaccgttgccgccccgtccgtgttcatcttccctccgagcgatgagcaacttaaaagtggaactgcaagcgttgtatgtcttctgaacaatttctatccccgagaagccaaggtacagtggaaagtggataatgccctccaatctggcaatagccaagagtctgtcacagagcaggacagcaaggactcaacttattcacttagctccaccctgacgctgagtaaagcagactacgagaagcataaggtgtatgcttgtgaggttacacaccaaggcttgtcttctcctgtcacgaagtctttcaataggggcgaatgctga

SEQ ID NO: 31 - protein, 234 aa, synthetic construct
MMSFVSLLLVGILFHATQAEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - DNA, 2385 bp, synthetic construct
  source 1..2385: mol_type = other DNA
atgatgtcctttgtctctctgctcctggttggcatcctattccatgccacccaggcccaagtccagctcgtggaatcaggagggggtgtagtccaaccagggcggagcctccgacttgattgtaaagcatcaggaattacattttctaatagcggaatgcattgggtccgacaagcgccaggcaagggactggaatgggtcgcggtgatatggtatgatggatcaaaacgctattatgcggactctgtgaagggtcgattcactattagcagagataacagcaagaatactctcttccttcagatgaattcacttagggcagaggacacagcggtgtactattgcgcgacgaacgacgattattggggccaagggacattggtaacggtgagttctgctagtactaaagggccttccgtcttcccactcgccccttgttctagaagtactagtgagtcaacagctgctttgggttgcttggttaaagactactttcctgaacccgtgactgtgtcttggaattccggtgctcttacttcaggtgttcatacattcccagcagtattgcagagctctggcttgtattctctctcctcagtggtgacagtaccttcctcctctcttggcacaaaaacttacacatgtaatgtagaccataaaccatcaaacacgaaagttgacaagagagtagaaagcaagtatgggcctccatgtcccccgtgcccggcgcccgagttcctgggtggtccgtcagtgttcttgttccctcccaagccaaaagatacattgatgataagtcggacgccggaggtcacatgtgtagtagttgatgtctctcaggaggatcctgaggtgcagtttaactggtacgtcgatggtgttgaggtacacaacgccaaaactaagccgagggaagagcagttcaattcaacatatcgggtcgtgtccgtattgacagttctgcaccaagattggttgaacggaaaagagtataagtgcaaagttagcaataagggacttccgtcctcaattgaaaaaaccatttccaaagcgaaaggccaacctcgggaacctcaggtatataccttgccacccagccaagaagaaatgactaaaaaccaggttagtttgacatgtttggttaaaggcttttacccgtccgacattgccgtcgagtgggaaagcaatgggcagcctgaaaataactacaagacaaccccaccagtattggattccgacggttccttctttctttacagccgcctcaccgtcgataagagtcggtggcaagaggggaatgtcttttcctgtagtgtcatgcacgaagcacttcacaaccattacacccaaaaatcattgtccctgtcactggggaaatccgggggtggcggatccgatgaggctgcagggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtga

SEQ ID NO: 35 - protein, 794 aa, synthetic construct
MMSFVSLLLVGILFHATQAQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSDEAAGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 36 - DNA, 702 bp, synthetic construct
  source 1..702: mol_type = other DNA
atgatgtcctttgtctctctgctcctggttggcatcctattccatgccacccaggccgaaatcgtattgactcagtcccctgctacactttcactgagtccgggtgagcgggcgactttgtcatgtcgggcatcacagagtgtaagtagttatctggcctggtatcaacagaaaccgggacaggctcctcgcctgctgatttatgacgcaagcaatcgcgcgaccggcatcccggcgaggttctcagggtctggatcaggtactgactttacccttacgatctcttctctcgaacctgaggatttcgctgtctattactgccagcagtcttctaactggccgagaacatttggtcaagggacaaaagtcgagattaagcgaactgtcgcagcgccatctgtctttatcttccctccaagcgacgaacagcttaagagtggcaccgccagtgttgtctgccttctgaataacttctatccaagggaagcgaaagttcagtggaaggtggataacgctctgcagtctgggaactctcaggaaagtgtaacagaacaagactccaaagactcaacctactctcttagttccacgttgaccctctccaaagcggactatgagaagcataaggtctacgcttgcgaggtaacacaccaggggctgagtagtccggttacgaagagcttcaacagaggggaatgctga

SEQ ID NO: 37 - protein, 233 aa, synthetic construct
MMSFVSLLLVGILFHATQAEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 2406 bp, synthetic construct
  source 1..2406: mol_type = other DNA
atgatgtcctttgtctctctgctcctggttggcatcctattccatgccacccaggcccaggtgcaattggtccaaagtggggtcgaggtcaagaagccaggagcttctgtaaaagtttcatgtaaggcatctgggtataccttcacgaactactatatgtattgggtgcgccaagcgccagggcagggcctcgaatggatgggtggcatcaatccgagcaacgggggcaccaactttaacgaaaagtttaaaaaccgggtcaccttgacaacggacagtagcacgactaccgcttatatggagctgaagagtttgcagtttgatgatactgcggtttattattgtgcacgcagagattataggttcgacatgggcttcgactactggggtcaaggtactacggtaactgtatcatctgctagtacaaagggcccttccgttttccccctcgccccgtgcagccgctcaacgtccgaaagcaccgctgcacttgggtgccttgtaaaagactattttccagagccagttaccgtgtcttggaatagtggcgcacttacgtccggggtgcacacttttccggctgtcttgcaatcctctggactctattccttgagtagcgtcgtaacagtaccaagtagtagtctcggcaccaaaacgtatacgtgcaatgttgatcataagcctagcaacacgaaagttgacaaaagagttgagagtaaatatggacccccctgtccgccatgcccggcccctgaattccttgggggcccgtctgtatttcttttcccgcccaagccgaaggatacactgatgataagcagaacgcctgaggttacctgcgtcgtggtcgacgtaagccaggaagatcctgaggtgcaatttaattggtatgtggacggggtcgaggttcataatgcaaaaacaaaaccccgagaggagcaatttaattcaacgtacagagtcgttagcgtacttacagtgctgcaccaggattggctcaacgggaaggagtataagtgcaaggtgtctaataaaggtttgccctccagtatagaaaaaaccatctcaaaggcgaaaggacagcctagagaacctcaagtatataccctcccaccctcccaagaagagatgacaaagaaccaagtgagtctcacatgcctcgtcaagggtttctacccaagcgatatagccgtagagtgggaatcaaatggtcagccggagaataactacaaaactactccgccagtcttggatagcgacgggtcttttttcctgtactctaggctgacggtggacaagtcaagatggcaggaaggaaatgtttttagctgctccgttatgcatgaggctctccacaatcattatacacaaaaaagtttgtctctgtcattggggaaatccgggggtggcggatccgatgaggctgcagggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtga

SEQ ID NO: 41 - protein, 801 aa, synthetic construct
MMSFVSLLLVGILFHATQAQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSDEAAGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 42 - DNA, 714 bp, synthetic construct
  source 1..714: mol_type = other DNA
atgatgtcctttgtctctctgctcctggttggcatcctattccatgccacccaggccgaaattgttcttacgcagagtccagcaactttgtcactctctcccggcgaacgagcgacattgtcctgtcgcgcgagtaagggtgtctcaacatctggatactcatatctgcattggtaccagcaaaaaccgggacaagcgccgcgattgctgatttatctcgcctcctaccttgaaagtggtgtgcctgcgaggttctccggtagtggatcaggcaccgatttcactttgaccatcagcagcctcgaaccagaagattttgccgtctactactgccaacatagcagggatttgccactgacattcggcgggggtacgaaagttgagattaaacggactgtagcggcaccttctgtcttcatcttcccaccaagcgatgagcagcttaaaagcggtacagcttcagtggtgtgccttttgaacaacttttatccgcgagaagccaaggtccaatggaaggtggataacgctttgcaatccggtaactcacaggagtcagtaacagagcaagatagtaaagatagcacgtattcacttagcagtacccttactcttagcaaggctgattatgaaaaacataaggtatatgcgtgcgaggtaacgcaccaaggacttagctccccagtgacgaagtcatttaaccggggggagtgctga

SEQ ID NO: 43 - protein, 237 aa, synthetic construct
MMSFVSLLLVGILFHATQAEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 329 aa, synthetic construct; identical to SEQ ID NO: 7

SEQ ID NO: 47 - protein, 14 aa, synthetic construct
GPFQQRGLFDFMLE

SEQ ID NO: 48 - DNA, 42 bp, synthetic construct
  source 1..42: mol_type = other DNA
ggcccgtttcaacagagaggcttatttgactttatgctagaa

SEQ ID NO: 49 - DNA, 990 bp, synthetic construct
  source 1..990: mol_type = other DNA
gatgaggctgcagggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtga

SEQ ID NO: 50 - DNA, 2415 bp, synthetic construct; identical to SEQ ID NO: 22
  source 1..2415: mol_type = other DNA

SEQ ID NO: 51 - protein, 804 aa, synthetic construct; identical to SEQ ID NO: 23

SEQ ID NO: 52 - DNA, 708 bp, synthetic construct; identical to SEQ ID NO: 24
  source 1..708: mol_type = other DNA

SEQ ID NO: 53 - protein, 235 aa, synthetic construct; identical to SEQ ID NO: 25

SEQ ID NO: 54 - DNA, 3420 bp, synthetic construct
  source 1..3420: mol_type = other DNA
atgatgtcctttgtctctctgctcctggttggcatcctattccatgccacccaggccgaggtacagcttttggagtcaggcggggggctcgtccaacctggggggtcactccggttgtcatgtgctgccagtggcttcacattctcatcttacattatgatgtgggttcgacaggcccctgggaaggggctggagtgggtttcctccatctacccctccgggggaattaccttctatgccgacacggtaaagggtcgcttcactataagtcgagataacagcaaaaatacgctgtatctccagatgaactctctcagggctgaggacacagctgtatattactgcgcgcggattaagttggggaccgtcacaacagtggattactggggtcaaggcactctggtaaccgtatcctcagcatccaccaagggcccaagtgtattcccgctggccccttcaagtaaatccacgtctggcggcacagccgctctcggttgcctggttaaggactacttcccagaacctgtcactgtcagttggaactcaggcgcattgacatctggtgtccatacattccccgcagtcctgcaaagctctggactttacagtcttagtagcgtagtgacagtcccatcttcaagtcttgggacccaaacttatatttgcaacgtaaatcataaaccctccaacactaaagtagacaaaaaagtagagccgaaatcttgcgacaaaacgcatacatgcccaccatgtcccgctccggaactcctgggcggcccgtccgtttttctctttcccccaaagcccaaggatacgcttatgatcagcagaacaccggaagttacttgtgtagtcgttgacgtgtctcacgaagatcccgaagtcaaatttaattggtatgtggatggcgtcgaagtgcacaacgcaaaaaccaaacccagagaggaacagtataacagcacgtatcgagtggtctccgtacttacggtcctccaccaggactggttgaatggcaaggagtacaagtgcaaagtgagcaataaagcgttgccagccccgatcgaaaaaaccatcagcaaggccaagggacagcctagagagccgcaggtttacaccttgccgccatcaagggatgaactgactaaaaaccaggtatccctgacctgcctggttaagggtttttaccccagtgatatagcggttgaatgggagtctaacgggcagccagagaacaactacaaaacgacacctcccgttctggattccgatggcagctttttcttgtattctaaactcaccgtggataaatcccgatggcagcaaggcaacgtcttctcctgcagcgtgatgcatgaagccttgcacaaccactatacccaaaagagtctcagcctgtcacccgggaaatccgggggtggcggatccgatgaggctgcagggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtccgggggtggcggatccgatgaggctgcagggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtga

SEQ ID NO: 55 - protein, 1139 aa, synthetic construct
MMSFVSLLLVGILFHATQAEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSDEAAGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYKSGGGGSDEAAGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 56 - DNA, 2373 bp, synthetic construct
  source 1..2373: mol_type = other DNA
atgatgtcctttgtctctctgctcctggttggcatcctattccatgccacccaggccgaggtacagcttttggagtcaggcggggggctcgtccaacctggggggtcactccggttgtcatgtgctgccagtggcttcacattctcatcttacattatgatgtgggttcgacaggcccctgggaaggggctggagtgggtttcctccatctacccctccgggggaattaccttctatgccgacacggtaaagggtcgcttcactataagtcgagataacagcaaaaatacgctgtatctccagatgaactctctcagggctgaggacacagctgtatattactgcgcgcggattaagttggggaccgtcacaacagtggattactggggtcaaggcactctggtaaccgtatcctcagcatccaccaagggcccaagtgtattcccgctggccccttcaagtaaatccacgtctggcggcacagccgctctcggttgcctggttaaggactacttcccagaacctgtcactgtcagttggaactcaggcgcattgacatctggtgtccatacattccccgcagtcctgcaaagctctggactttacagtcttagtagcgtagtgacagtcccatcttcaagtcttgggacccaaacttatatttgcaacgtaaatcataaaccctccaacactaaagtagacaaaaaagtagagccgaaatcttgcgacaaaacgcatacatgcccaccatgtcccgctccggaactcctgggcggcccgtccgtttttctctttcccccaaagcccaaggatacgcttatgatcagcagaacaccggaagttacttgtgtagtcgttgacgtgtctcacgaagatcccgaagtcaaatttaattggtatgtggatggcgtcgaagtgcacaacgcaaaaaccaaacccagagaggaacagtataacagcacgtatcgagtggtctccgtacttacggtcctccaccaggactggttgaatggcaaggagtacaagtgcaaagtgagcaataaagcgttgccagccccgatcgaaaaaaccatcagcaaggccaagggacagcctagagagccgcaggtttacaccttgccgccatcaagggatgaactgactaaaaaccaggtatccctgacctgcctggttaagggtttttaccccagtgatatagcggttgaatgggagtctaacgggcagccagagaacaactacaaaacgacacctcccgttctggattccgatggcagctttttcttgtattctaaactcaccgtggataaatcccgatggcagcaaggcaacgtcttctcctgcagcgtgatgcatgaagccttgcacaaccactatacccaaaagagtctcagcctgtcacccgggaaatccgggggtggcggatccgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtga

SEQ ID NO: 57 - protein, 790 aa, synthetic construct
MMSFVSLLLVGILFHATQAEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 58 - DNA, 3336 bp, synthetic construct
  source 1..3336: mol_type = other DNA
atgatgtcctttgtctctctgctcctggttggcatcctattccatgccacccaggccgaggtacagcttttggagtcaggcggggggctcgtccaacctggggggtcactccggttgtcatgtgctgccagtggcttcacattctcatcttacattatgatgtgggttcgacaggcccctgggaaggggctggagtgggtttcctccatctacccctccgggggaattaccttctatgccgacacggtaaagggtcgcttcactataagtcgagataacagcaaaaatacgctgtatctccagatgaactctctcagggctgaggacacagctgtatattactgcgcgcggattaagttggggaccgtcacaacagtggattactggggtcaaggcactctggtaaccgtatcctcagcatccaccaagggcccaagtgtattcccgctggccccttcaagtaaatccacgtctggcggcacagccgctctcggttgcctggttaaggactacttcccagaacctgtcactgtcagttggaactcaggcgcattgacatctggtgtccatacattccccgcagtcctgcaaagctctggactttacagtcttagtagcgtagtgacagtcccatcttcaagtcttgggacccaaacttatatttgcaacgtaaatcataaaccctccaacactaaagtagacaaaaaagtagagccgaaatcttgcgacaaaacgcatacatgcccaccatgtcccgctccggaactcctgggcggcccgtccgtttttctctttcccccaaagcccaaggatacgcttatgatcagcagaacaccggaagttacttgtgtagtcgttgacgtgtctcacgaagatcccgaagtcaaatttaattggtatgtggatggcgtcgaagtgcacaacgcaaaaaccaaacccagagaggaacagtataacagcacgtatcgagtggtctccgtacttacggtcctccaccaggactggttgaatggcaaggagtacaagtgcaaagtgagcaataaagcgttgccagccccgatcgaaaaaaccatcagcaaggccaagggacagcctagagagccgcaggtttacaccttgccgccatcaagggatgaactgactaaaaaccaggtatccctgacctgcctggttaagggtttttaccccagtgatatagcggttgaatgggagtctaacgggcagccagagaacaactacaaaacgacacctcccgttctggattccgatggcagctttttcttgtattctaaactcaccgtggataaatcccgatggcagcaaggcaacgtcttctcctgcagcgtgatgcatgaagccttgcacaaccactatacccaaaagagtctcagcctgtcacccgggaaatccgggggtggcggatccgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtccgggggtggcggatccgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtga

SEQ ID NO: 59 - protein, 1111 aa, synthetic construct
MMSFVSLLLVGILFHATQAEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYKSGGGGSDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 60 - DNA, 1746 bp, synthetic construct
  source 1..1746: mol_type = other DNA
atgatgtcctttgtctctctgctcctggttggcatcctattccatgccacccaggccgaggtacagcttttggagtcaggcggggggctcgtccaacctggggggtcactccggttgtcatgtgctgccagtggcttcacattctcatcttacattatgatgtgggttcgacaggcccctgggaaggggctggagtgggtttcctccatctacccctccgggggaattaccttctatgccgacacggtaaagggtcgcttcactataagtcgagataacagcaaaaatacgctgtatctccagatgaactctctcagggctgaggacacagctgtatattactgcgcgcggattaagttggggaccgtcacaacagtggattactggggtcaaggcactctggtaaccgtatcctcagcatccaccaagggcccaagtgtattcccgctggccccttcaagtaaatccacgtctggcggcacagccgctctcggttgcctggttaaggactacttcccagaacctgtcactgtcagttggaactcaggcgcattgacatctggtgtccatacattccccgcagtcctgcaaagctctggactttacagtcttagtagcgtagtgacagtcccatcttcaagtcttgggacccaaacttatatttgcaacgtaaatcataaaccctccaacactaaagtagacaaaaaagtagagccgaaatcttgcgacaaaacgcatacatgcccaccatgtcccgctccggaactcctgggcggcccgtccgtttttctctttcccccaaagcccaaggatacgcttatgatcagcagaacaccggaagttacttgtgtagtcgttgacgtgtctcacgaagatcccgaagtcaaatttaattggtatgtggatggcgtcgaagtgcacaacgcaaaaaccaaacccagagaggaacagtataacagcacgtatcgagtggtctccgtacttacggtcctccaccaggactggttgaatggcaaggagtacaagtgcaaagtgagcaataaagcgttgccagccccgatcgaaaaaaccatcagcaaggccaagggacagcctagagagccgcaggtttacaccttgccgccatcaagggatgaactgactaaaaaccaggtatccctgacctgcctggttaagggtttttaccccagtgatatagcggttgaatgggagtctaacgggcagccagagaacaactacaaaacgacacctcccgttctggattccgatggcagctttttcttgtattctaaactcaccgtggataaatcccgatggcagcaaggcaacgtcttctcctgcagcgtgatgcatgaagccttgcacaaccactatacccaaaagagtctcagcctgtcacccgggaaatccgggggtggcggatccctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgtttga

SEQ ID NO: 61 - protein, 581 aa, synthetic construct
MMSFVSLLLVGILFHATQAEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSLRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAV

SEQ ID NO: 62 - DNA, 2082 bp, synthetic construct
  source 1..2082: mol_type = other DNA
atgatgtcctttgtctctctgctcctggttggcatcctattccatgccacccaggccgaggtacagcttttggagtcaggcggggggctcgtccaacctggggggtcactccggttgtcatgtgctgccagtggcttcacattctcatcttacattatgatgtgggttcgacaggcccctgggaaggggctggagtgggtttcctccatctacccctccgggggaattaccttctatgccgacacggtaaagggtcgcttcactataagtcgagataacagcaaaaatacgctgtatctccagatgaactctctcagggctgaggacacagctgtatattactgcgcgcggattaagttggggaccgtcacaacagtggattactggggtcaaggcactctggtaaccgtatcctcagcatccaccaagggcccaagtgtattcccgctggccccttcaagtaaatccacgtctggcggcacagccgctctcggttgcctggttaaggactacttcccagaacctgtcactgtcagttggaactcaggcgcattgacatctggtgtccatacattccccgcagtcctgcaaagctctggactttacagtcttagtagcgtagtgacagtcccatcttcaagtcttgggacccaaacttatatttgcaacgtaaatcataaaccctccaacactaaagtagacaaaaaagtagagccgaaatcttgcgacaaaacgcatacatgcccaccatgtcccgctccggaactcctgggcggcccgtccgtttttctctttcccccaaagcccaaggatacgcttatgatcagcagaacaccggaagttacttgtgtagtcgttgacgtgtctcacgaagatcccgaagtcaaatttaattggtatgtggatggcgtcgaagtgcacaacgcaaaaaccaaacccagagaggaacagtataacagcacgtatcgagtggtctccgtacttacggtcctccaccaggactggttgaatggcaaggagtacaagtgcaaagtgagcaataaagcgttgccagccccgatcgaaaaaaccatcagcaaggccaagggacagcctagagagccgcaggtttacaccttgccgccatcaagggatgaactgactaaaaaccaggtatccctgacctgcctggttaagggtttttaccccagtgatatagcggttgaatgggagtctaacgggcagccagagaacaactacaaaacgacacctcccgttctggattccgatggcagctttttcttgtattctaaactcaccgtggataaatcccgatggcagcaaggcaacgtcttctcctgcagcgtgatgcatgaagccttgcacaaccactatacccaaaagagtctcagcctgtcacccgggaaatccgggggtggcggatccctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgtttccgggggtggcggatccctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgtttga

SEQ ID NO: 63 - protein, 693 aa, synthetic construct
MMSFVSLLLVGILFHATQAEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSLRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVSGGGGSLRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAV

SEQ ID NO: 64 - protein, 315 aa, synthetic construct
DFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 65 - protein, 636 aa, synthetic construct
DFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYKSGGGGSDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 66 - protein, 106 aa, synthetic construct
LRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAV

SEQ ID NO: 67 - protein, 218 aa, synthetic construct
LRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVSGGGGSLRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAV